SEQUENCE LISTING (100 sequences)

SEQ ID NO: 1 - DNA, 2090 bp, Zea mays
aaaaatttcaatgcctctctagatctttcaagctatccttgcttcctagctccctttggcgtgctgtaccggacgacaggggtaggtaggtaggtaggtgtagctagctcggcgagcgagggagcaatccggccaacagcacacgcgtcgatcgccatgccgagaagcggcggcggcggcaatggcggtggcattagtagtagtagtagggtgatgcccaacctcccggccgggttccgcttccaccccacggacgaggagctcatggtgcactacctcatgaggcaggccgcctccatgccgtgccccgtccccatcatcgccgaggtcaacatctaccagtgcaacccctgggatctccctggtacgtgattctttatttctttcttccctctcttattcgttcattgtcacttgagttgagtcgttcagactagttgtgttattcctagctaatccaaatctgcacccagttgaatctcatatgatttaattattctttcttcctcctgtggtatcgtgttcgttcttcagccaaggcattgttcggcgacaaggagtggtttttcttcagcccccgggaccgcaagtaccccaacggcgcccgccccaaccgcgccgccgggtccgggtactggaaggccaccggcaccgacaaggccatcctgtcgtcgtccacgcccacgagccacggcggcgccaacatcgtcgtcggcgtcaagaaggcgctcgtcttctacggcggcaggccgcccaagggcaccaagacggactggatcatgcacgagtaccgcctctcgggcgcggcggacgacgactgcaagggcagcaccaggcgcagagtgtcttcctcctcgtcgtcctccatgagggtgagtgagactttctttttctttttcttcttttttttttaattgcacttgcttgcacggctccagacctgactgaagcgtttcgcatggtttcagctggacgactgggtgctgtgcaggatccacaagaagagcaacgatttccagttgtcgtcgtcgtcggagcatgagcacgagcaggaggagccggcggcgggcggctcagcgacgactgtggaggaactagacgctctcgtcgtcgtcgacaacagcagcagcagcgacaccaccaccaccaccatcaccaccaacgacaacaactctactgaaacagctgcgactcatcacgatcctcagacgatgatgatgctgagcaagtcgtgctcgctcaccgacctcctcgacagcatcgactacgcggcgctctcctcgcagatgctcctcctcgacgcgccccacccccacgctgatgacccgccgccacacatggtctactacccaccggcggcgtcggcgacgcaccagcaagcaataatagctaataacaccaacagtaatgacgactactatgacgacgacaacttgttgccgactgctgctgctactgcggtagacgcggcggcggcggcggtgctactcagctcgtcgcctgcagacaccaacggtgtgaacaagaggaagcgagtgacggccgtggactactacggcgccgctgaaccgccgaccttcttccaccaccacctcgatgatggcggcaacagcttcttcagtacgaccaagaagctcaagccgccgccgccaagtgattcgaggcatggcggccatttcggcaccgccacagcgagcagctaccgcgacagccagcagctcgtggacagcgcgagcagcagcggcgtcttcttccatcagtatgggtacggctacagcagcagcaacccgttcttgaacttgaaccagcagcagcagctgctactcaacagccacatcgggatgcagtagagctcgagagatcgagagagagagagagagagagatgatatatcattggaaggatcagcacgcgtacgtgcagattaattgcagcgagttctatcttgtttgctttcattattatatactccgtatgtgtcgttcatgtaaaattagtttaaattgtaaattagtgtgctgaaattcatgtgcctgctctctgctcatcaatgaatggatgatgacgacccatgcacgttgcttgtgtgg

SEQ ID NO: 2 - DNA, 1428 bp, Zea mays
atgccgagaagcggcggcggcggcaatggcggtggcattagtagtagtagtagggtgatgcccaacctcccggccgggttccgcttccaccccacggacgaggagctcatggtgcactacctcatgaggcaggccgcctccatgccgtgccccgtccccatcatcgccgaggtcaacatctaccagtgcaacccctgggatctccctgccaaggcattgttcggcgacaaggagtggtttttcttcagcccccgggaccgcaagtaccccaacggcgcccgccccaaccgcgccgccgggtccgggtactggaaggccaccggcaccgacaaggccatcctgtcgtcgtccacgcccacgagccacggcggcgccaacatcgtcgtcggcgtcaagaaggcgctcgtcttctacggcggcaggccgcccaagggcaccaagacggactggatcatgcacgagtaccgcctctcgggcgcggcggacgacgactgcaagggcagcaccaggcgcagagtgtcttcctcctcgtcgtcctccatgaggctggacgactgggtgctgtgcaggatccacaagaagagcaacgatttccagttgtcgtcgtcgtcggagcatgagcacgagcaggaggagccggcggcgggcggctcagcgacgactgtggaggaactagacgctctcgtcgtcgtcgacaacagcagcagcagcgacaccaccaccaccaccatcaccaccaacgacaacaactctactgaaacagctgcgactcatcacgatcctcagacgatgatgatgctgagcaagtcgtgctcgctcaccgacctcctcgacagcatcgactacgcggcgctctcctcgcagatgctcctcctcgacgcgccccacccccacgctgatgacccgccgccacacatggtctactacccaccggcggcgtcggcgacgcaccagcaagcaataatagctaataacaccaacagtaatgacgactactatgacgacgacaacttgttgccgactgctgctgctactgcggtagacgcggcggcggcggcggtgctactcagctcgtcgcctgcagacaccaacggtgtgaacaagaggaagcgagtgacggccgtggactactacggcgccgctgaaccgccgaccttcttccaccaccacctcgatgatggcggcaacagcttcttcagtacgaccaagaagctcaagccgccgccgccaagtgattcgaggcatggcggccatttcggcaccgccacagcgagcagctaccgcgacagccagcagctcgtggacagcgcgagcagcagcggcgtcttcttccatcagtatgggtacggctacagcagcagcaacccgttcttgaacttgaaccagcagcagcagctgctactcaacagccacatcgggatgcagtag

SEQ ID NO: 3 - DNA, 1824 bp, Artificial Sequence
  cDNA
aaaaatttcaatgcctctctagatctttcaagctatccttgcttcctagctccctttggcgtgctgtaccggacgacaggggtaggtaggtaggtaggtgtagctagctcggcgagcgagggagcaatccggccaacagcacacgcgtcgatcgccatgccgagaagcggcggcggcggcaatggcggtggcattagtagtagtagtagggtgatgcccaacctcccggccgggttccgcttccaccccacggacgaggagctcatggtgcactacctcatgaggcaggccgcctccatgccgtgccccgtccccatcatcgccgaggtcaacatctaccagtgcaacccctgggatctccctgccaaggcattgttcggcgacaaggagtggtttttcttcagcccccgggaccgcaagtaccccaacggcgcccgccccaaccgcgccgccgggtccgggtactggaaggccaccggcaccgacaaggccatcctgtcgtcgtccacgcccacgagccacggcggcgccaacatcgtcgtcggcgtcaagaaggcgctcgtcttctacggcggcaggccgcccaagggcaccaagacggactggatcatgcacgagtaccgcctctcgggcgcggcggacgacgactgcaagggcagcaccaggcgcagagtgtcttcctcctcgtcgtcctccatgaggctggacgactgggtgctgtgcaggatccacaagaagagcaacgatttccagttgtcgtcgtcgtcggagcatgagcacgagcaggaggagccggcggcgggcggctcagcgacgactgtggaggaactagacgctctcgtcgtcgtcgacaacagcagcagcagcgacaccaccaccaccaccatcaccaccaacgacaacaactctactgaaacagctgcgactcatcacgatcctcagacgatgatgatgctgagcaagtcgtgctcgctcaccgacctcctcgacagcatcgactacgcggcgctctcctcgcagatgctcctcctcgacgcgccccacccccacgctgatgacccgccgccacacatggtctactacccaccggcggcgtcggcgacgcaccagcaagcaataatagctaataacaccaacagtaatgacgactactatgacgacgacaacttgttgccgactgctgctgctactgcggtagacgcggcggcggcggcggtgctactcagctcgtcgcctgcagacaccaacggtgtgaacaagaggaagcgagtgacggccgtggactactacggcgccgctgaaccgccgaccttcttccaccaccacctcgatgatggcggcaacagcttcttcagtacgaccaagaagctcaagccgccgccgccaagtgattcgaggcatggcggccatttcggcaccgccacagcgagcagctaccgcgacagccagcagctcgtggacagcgcgagcagcagcggcgtcttcttccatcagtatgggtacggctacagcagcagcaacccgttcttgaacttgaaccagcagcagcagctgctactcaacagccacatcgggatgcagtagagctcgagagatcgagagagagagagagagagagatgatatatcattggaaggatcagcacgcgtacgtgcagattaattgcagcgagttctatcttgtttgctttcattattatatactccgtatgtgtcgttcatgtaaaattagtttaaattgtaaattagtgtgctgaaattcatgtgcctgctctctgctcatcaatgaatggatgatgacgacccatgcacgttgcttgtgtgg

SEQ ID NO: 4 - protein, 475 aa, Artificial Sequence
  ZmNAC111
MPRSGGGGNGGGISSSSRVMPNLPAGFRFHPTDEELMVHYLMRQAASMPCPVPIIAEVNIYQCNPWDLPAKALFGDKEWFFFSPRDRKYPNGARPNRAAGSGYWKATGTDKAILSSSTPTSHGGANIVVGVKKALVFYGGRPPKGTKTDWIMHEYRLSGAADDDCKGSTRRRVSSSSSSSMRLDDWVLCRIHKKSNDFQLSSSSEHEHEQEEPAAGGSATTVEELDALVVVDNSSSSDTTTTTITTNDNNSTETAATHHDPQTMMMLSKSCSLTDLLDSIDYAALSSQMLLLDAPHPHADDPPPHMVYYPPAASATHQQAIIANNTNSNDDYYDDDNLLPTAAATAVDAAAAAVLLSSSPADTNGVNKRKRVTAVDYYGAAEPPTFFHHHLDDGGNSFFSTTKKLKPPPPSDSRHGGHFGTATASSYRDSQQLVDSASSSGVFFHQYGYGYSSSNPFLNLNQQQQLLLNSHIGMQ

SEQ ID NO: 5 - DNA, 1176 bp, Oryza sativa
atgccgagcagcggcggcgccatgcctgcccttccaccaggcttccgcttccaccccaccgacgaggagctcatcgttcactacctcatgaaccaggccgcctccgtcaagtgccccgtgccaatcatcgccgaggtcaacatctacaagtgcaacccatgggaccttcctggtaaggctttgttcggcgagaacgaatggtacttcttcagcccgagggaccgcaagtaccccaacggcgctcgccccaaccgcgccgccggctcggggtactggaaggccaccggcaccgacaagtccatcctctccactccgaccagcgacaacatcggcgtcaagaaggccctcgtcttctacaagggcaagcctcccaagggcgtcaagaccgactggatcatgcacgagtaccgtctcaccggcacatcagctaacagcaccaccaccacaaagcagcgtagagcgtcatccatgaccatgaggctggacgactgggtgctgtgcagaatccacaagaagagcaacgacttcaattcctctgaccaacacgaccaagaacccgaggaatcaaccgtcgaacagcttgaagacatccatgacaacaactcctctgaacaacctccagctccagctgacatgaacaaccaacagtcagatttccagcccatgacggcgatgagcatgagcaagtcatgctccctcaccgatctcctcaacaccatcgactgcgccgcgctctcgcagtttctcctcgacggctcatccgacgccatcgctgagcctcctgctcctcccagccccctaatatacacaacacctcatccaaattaccaaacactaaactataacattaacagcaacagcagcatgccacacgccttcgagtcacgcctagatcatcacgatggttacgttaacaattataatgttaatggcctgaggaggaagagaatgatggcgtgtagtgcaacttcctttgatgatggcagcagcagcaatgactttgtgcatgccgttgtcaagaaaccgcagctgctgccaagtgattcgaggggtagtggttttggaggaggttactgcaaccagcagctttcagagactgcgactggctttcagtttcagaacggcaatctgctgagccatccatttcctctgaacaatcatctgcagatgcagtag

SEQ ID NO: 6 - protein, 391 aa, Oryza sativa
MPSSGGAMPALPPGFRFHPTDEELIVHYLMNQAASVKCPVPIIAEVNIYKCNPWDLPGKALFGENEWYFFSPRDRKYPNGARPNRAAGSGYWKATGTDKSILSTPTSDNIGVKKALVFYKGKPPKGVKTDWIMHEYRLTGTSANSTTTTKQRRASSMTMRLDDWVLCRIHKKSNDFNSSDQHDQEPEESTVEQLEDIHDNNSSEQPPAPADMNNQQSDFQPMTAMSMSKSCSLTDLLNTIDCAALSQFLLDGSSDAIAEPPAPPSPLIYTTPHPNYQTLNYNINSNSSMPHAFESRLDHHDGYVNNYNVNGLRRKRMMACSATSFDDGSSSNDFVHAVVKKPQLLPSDSRGSGFGGGYCNQQLSETATGFQFQNGNLLSHPFPLNNHLQMQ

SEQ ID NO: 7 - DNA, 1032 bp, Glycine max
atgggagttccagagaaagaccctctttcccaattgagtttgcctcctggttttcggttttaccccaccgacgaggagcttctcgttcagtatctgtgccgcaaggtcgctggccaccatttctctcttccaatcattgccgaaattgacttgtacaagttcgacccatgggttcttccaagtaaagcgattttcggtgaaaaagagtggtactttttcagccccagagacaggaaatacccgaacgggtctcgacccaacagagtagctgggtcgggttattggaaagccaccggaaccgacaagatcatcaccaccgaaggtagaaaagttggcataaaaaaagccctcgttttctacgttggcaaagcccccaagggcaccaaaaccaattggatcatgcacgagtatcgcctcctcgactcttcccgaaagaacactggcaccaagcttgatgattgggttctgtgtcgtatatacaagaagaactcgagtgcacagaagacggcgcaaaacggcgtggttccgagcaacgagcacactcaatacagcaacggttcctcttcttcttcttcgtcccagctggaggacgttctggaatctctgccatcgattgatgaaaggtgtttcgcgatgccacgcgtcaacacgctgcaacaacaacagcaccacgaggagaaggtcaatgttcagaacttgggtgcagggggtttaatggattggaccaacccttcggttctgaattcggtcgccgatttcgcttcggggaataatcaagtggtacaggaccagactcaggggatggtgaactacaactgcaatgacctttatgtccctacgttatgccacttggactcatcggttccgttaaagatggaggaagaggtgcaaagcggcgtgagaaaccaacgggtcgggaataataattcgtggtttcttcagaatgatttcacacaggggtttcagaattcggttgacacgtgtgggtttaaatacccggttcagccggtcgggttcgggttcagaaattga

SEQ ID NO: 8 - protein, 343 aa, Glycine max
MGVPEKDPLSQLSLPPGFRFYPTDEELLVQYLCRKVAGHHFSLPIIAEIDLYKFDPWVLPSKAIFGEKEWYFFSPRDRKYPNGSRPNRVAGSGYWKATGTDKIITTEGRKVGIKKALVFYVGKAPKGTKTNWIMHEYRLLDSSRKNTGTKLDDWVLCRIYKKNSSAQKTAQNGVVPSNEHTQYSNGSSSSSSSQLEDVLESLPSIDERCFAMPRVNTLQQQQHHEEKVNVQNLGAGGLMDWTNPSVLNSVADFASGNNQVVQDQTQGMVNYNCNDLYVPTLCHLDSSVPLKMEEEVQSGVRNQRVGNNNSWFLQNDFTQGFQNSVDTCGFKYPVQPVGFGFRN

SEQ ID NO: 9 - DNA, 1290 bp, Sorghum bicolor
atggcgagtggtggcggcagtagcagtagagtgcccggcggcggtggcaacctgaacctcccagctgggttccggttccaccccactgacgaggagctgatcgtgcactacctcatgaaccaggccgcctccattccgtgccctgtccccatcatcgccgaggtcaacatctaccagtgcaacccctgggatctccctgcaaaagcattgtttggcgagaacgagtggtacttcttcagcccacgagaccgcaagtaccccaatggcgtccgccccaaccgcgccgccgggtcagggtactggaaggccaccggcaccgacaaggccatcctgtccacgccgacgagccagaacatcggcgtcaagaaggccctcgtcttctacggcggcaggcctcccaagggcgtcaagaccgactggatcatgcacgagtaccgcctcaccggcggcacgaccgccgcagcggccgatgacaagaacagcagcatcaaacgcagatcagggtcctccatgaggctggacgactgggtgctgtgcaggatccacaagaagagcaacgatttccagttgtcggaccaggagcaggagaaggagggctcaaccgtggaggaattagacactcttgccaacaccaccaacaccgacaacagcatgaacgactctactgaaacaacaactcttggtcatcatgaccagctccgtcatgaaacggcggccatgacgacgatgagcaagtcgtgctcgctcaccgacctcctcaacagcatcgactacgcgtcgctctcgcagatgttccttgacatccctgccgaggccgaggagcctgcacaacagcaaagcactccactaatctacccaccagcaacacaaacaacacaccaagcagcacttactagtaataataactatgacaacaacgtgatgaacaacagcaacttgccgattgctgcagtagacgcagtgatagcaggctcggataacagcggtgtgaagaagaggaataagagagtgatggccgtggacggcgccgccgctgaatcgtgcttcgatgatggcagcagcgacagtttcagtagcaagaaactgaagctgccaagtgattcgaggatcgccggccatttcggcaccacagcaagcagctactactactgcaacaaccagctgcagcttgtggacagcgcgatgagtagtggctttcatcagtacagcagcctgttgctgagcagcagcaatccgttcttgagccagcagcagcagctgctactcaacagccacatcgggatgcagtag

SEQ ID NO: 10 - protein, 429 aa, Sorghum bicolor
MASGGGSSSRVPGGGGNLNLPAGFRFHPTDEELIVHYLMNQAASIPCPVPIIAEVNIYQCNPWDLPAKALFGENEWYFFSPRDRKYPNGVRPNRAAGSGYWKATGTDKAILSTPTSQNIGVKKALVFYGGRPPKGVKTDWIMHEYRLTGGTTAAAADDKNSSIKRRSGSSMRLDDWVLCRIHKKSNDFQLSDQEQEKEGSTVEELDTLANTTNTDNSMNDSTETTTLGHHDQLRHETAAMTTMSKSCSLTDLLNSIDYASLSQMFLDIPAEAEEPAQQQSTPLIYPPATQTTHQAALTSNNNYDNNVMNNSNLPIAAVDAVIAGSDNSGVKKRNKRVMAVDGAAAESCFDDGSSDSFSSKKLKLPSDSRIAGHFGTTASSYYYCNNQLQLVDSAMSSGFHQYSSLLLSSSNPFLSQQQQLLLNSHIGMQ

SEQ ID NO: 11 - DNA, 747 bp, Gossypium raimondii
ttcagattccatcctactgatgaagagcttatcattcattatctaaaccagaaggttttcccttcctccaatcatcacctaatgttctccattatagctgatgttaatatctacaagttcaatccatgggaacttcctgataaggctttatttggtgagaacgagtggtttttctttagtccaagagaaaggaagtatccaaacggaacacgcccgaaccgagcagcggcatcgggatactggaaggctaccgggaccgataaaccgatcatcgcttctgttggttcacaatgccttggaatgaagaaagctttagtgttttacaaaggacgtcctcctaaaggtgtaaaaaccgattggatgatgattgaatatagactgctcgatgattgctttgtatctcaaagacctaaaggatcaatgcaattggatgattgggttctatgccgagttagtcacaaaggcaaagctccattggctggtgggtacttacaaggtcaagagatggcaatacaaatcaccattggaatcaatgaatatgatcatcagttacctacaatggaccagatgattggccttgaaagtgaagagctcgatgaaaaggtgtttcaagaagggtccccagagtatcctccaacacccatagataccagtgtgagagaggtgctaaagtccattgaaagggtactgtctgttggagctttggatgaactggtaatggcttcttcaagtgacgatgctgtgtga

SEQ ID NO: 12 - protein, 248 aa, Gossypium raimondii
FRFHPTDEELIIHYLNQKVFPSSNHHLMFSIIADVNIYKFNPWELPDKALFGENEWFFFSPRERKYPNGTRPNRAAASGYWKATGTDKPIIASVGSQCLGMKKALVFYKGRPPKGVKTDWMMIEYRLLDDCFVSQRPKGSMQLDDWVLCRVSHKGKAPLAGGYLQGQEMAIQITIGINEYDHQLPTMDQMIGLESEELDEKVFQEGSPEYPPTPIDTSVREVLKSIERVLSVGALDELVMASSSDDAV

SEQ ID NO: 13 - DNA, 978 bp, Brassica rapa
atgataagcaaggatccaagatcaagtttgccaccagggtttcgatttcatccaactgatgaagaactcattctccattacctaaggaagaaggtttcctctttaccagtcccgctttcggtcatcgaagatgtcgatatctataaatctgatccatgggatttaccagctaaggctccctttggagagaaagaatggtattttttcagtccgagggacaggaaatatccaaacggagcaagaccaaaccgagcagctgcgtcagggtattggaaagccaccggaacagataaattaattgcggtaccaaacggtggagttaatgaaaacattggtataaaaaaagctcttgtgttttatagaggaaagcctccaaaaggtgttaaaaccaattggatcatgcatgaatatcgacttgctgaaaccctatcgcccaaaagagtggaccattcgagcgacagccaattcaataatcttggagacaggagtttgaaatctagagaatactctatgaggctggatgattgggttctttgccggatttacaagaaatcacacatttcactgtcaccaccacatgttgctactgatacaagcaaccaagaacatgaggaaaatgacaaagaaccattcatagtcagcgaaaccctcttgccaaatttggaaaacaatcaaacacttaaacgccagaagtcttctttctcgaacttattagacgctacagatttgacgttcctcacaaactttctaaacgaaactccggaacatcatactgaacaagagttttctttcatgtttgaaaatttctcaaaccctaacatctacggaaacccttacttggatcaaaagttacctcggttaagccctcctagttctgagaccagctttatcggaaacaaaagagaaagaatggattatgcagaagaaacgacgagcacttccaagaagatgatcaacaactttagttacaatatataa

SEQ ID NO: 14 - protein, 325 aa, Brassica rapa
MISKDPRSSLPPGFRFHPTDEELILHYLRKKVSSLPVPLSVIEDVDIYKSDPWDLPAKAPFGEKEWYFFSPRDRKYPNGARPNRAAASGYWKATGTDKLIAVPNGGVNENIGIKKALVFYRGKPPKGVKTNWIMHEYRLAETLSPKRVDHSSDSQFNNLGDRSLKSREYSMRLDDWVLCRIYKKSHISLSPPHVATDTSNQEHEENDKEPFIVSETLLPNLENNQTLKRQKSSFSNLLDATDLTFLTNFLNETPEHHTEQEFSFMFENFSNPNIYGNPYLDQKLPRLSPPSSETSFIGNKRERMDYAEETTSTSKKMINNFSYNI

SEQ ID NO: 15 - DNA, 1065 bp, Triticum aestivum
atgccaatgggcagcagcgccgccatgcccgccctccctcccggcttccggttccaccccaccgacgaggagctcatcgtccactacctccgcaggcaggccgcgtccatgcccagccccgtgcccatcatcgccgaggtcaacatctacaagtgcaacccatgggacctccccggcaaagctttgttcggggagaatgagtggtacttcttcagcccccgggatcgcaagtaccccaacggcgcgcgcccgaaccgcgccgccgggtccggctactggaaggccaccggcaccgacaaggccatcctgtccacgccggccaacgagagcatcggggtcaagaaggcgctcgtgttctacaggggcaagccgcccaagggcgtcaagaccgactggatcatgcacgagtaccgcctcaccgcagccgacaaccggaccaccaagcgcagaggatcctccatgaggctggatgactgggtgctgtgtaggatccacaagaagtgcggcaacttgcccaacttctcctcctctgaccaggaacaggagcatgagcaggagagctccaccgtggaggactcgcagaacaaccacaccgtgtcgtcgcccaagtccgaggccttcgacggcgacggcgacgaccacctccagttgcagcagttccgccccatggcgatcgccaagtcgtgctccctcaccgacctgctcaacaccgtcgactacgccgcgctctcgcacctcctcctcgacggcgccggcgcctcgtcgtcggacgccggagcagactaccagctgcctcccgaaaacccactcatctactcgcagcctccatggcaacaaacgctacactataataacaacaacggctacgtgaacaacgagaccatcgacgtgcctcagctacccgaggcgcgcgtagatgactacggcatgaatggcgataagtataacggcatgaagaggaagagatccagcggcagcttgtactgcagccagctgcagctcccagcggatcagtacagcggcatgctgatccatccgttcctcagccagcagctgcacatgtga

SEQ ID NO: 16 - protein, 354 aa, Triticum aestivum
MPMGSSAAMPALPPGFRFHPTDEELIVHYLRRQAASMPSPVPIIAEVNIYKCNPWDLPGKALFGENEWYFFSPRDRKYPNGARPNRAAGSGYWKATGTDKAILSTPANESIGVKKALVFYRGKPPKGVKTDWIMHEYRLTAADNRTTKRRGSSMRLDDWVLCRIHKKCGNLPNFSSSDQEQEHEQESSTVEDSQNNHTVSSPKSEAFDGDGDDHLQLQQFRPMAIAKSCSLTDLLNTVDYAALSHLLLDGAGASSSDAGADYQLPPENPLIYSQPPWQQTLHYNNNNGYVNNETIDVPQLPEARVDDYGMNGDKYNGMKRKRSSGSLYCSQLQLPADQYSGMLIHPFLSQQLHM

SEQ ID NO: 17 - protein, 19 aa, Artificial Sequence
  sub-domain A
LPPGFRFHPTDEELICHYL

SEQ ID NO: 18 - protein, 21 aa, Artificial Sequence
  sub-domain B
IIAEVDLYKCEPWDLPEKCKI

SEQ ID NO: 19 - protein, 35 aa, Artificial Sequence
  sub-domain C
WYFFCPRDRKYPNGTRTNRATGSGYWKATGKDKEI

SEQ ID NO: 20 - protein, 29 aa, Artificial Sequence
  sub-domain D
VGMRKTLVFYMGRAPRGTKTNWVMHEFRL

SEQ ID NO: 21 - protein, 11 aa, Artificial Sequence
  sub-domain E
DEWVVCKVHHK

SEQ ID NO: 22 - protein, 172 aa, Artificial Sequence
  NAC DNA- binding domain
LPAGFRFHPTDEELMVHYLMRQAASMPCPVPIIAEVNIYQCNPWDLPAKALFGDKEWFFFSPRDRKYPNGARPNRAAGSGYWKATGTDKAILSSSTPTSHGGANIVVGVKKALVFYGGRPPKGTKTDWIMHEYRLSGAADDDCKGSTRRRVSSSSSSSMRLDDWVLCRIHKK

SEQ ID NO: 23 - DNA, 1996 bp, Artificial Sequence
  Zmubi1 promoter
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 24 - DNA, 1201 bp, Artificial Sequence
  ZmNac111 promoter
tctgaaagaaataaatattgctaagattttttctaaaatttagtcaaaatcaagaaagtttaaccatcatgaacatcgtagcgacactgattctaggtattaattaagtattcctacaggctagacgaagcaattaatatgagaaaggttgacggaggtccggggacgaaggtcataacccttgggaccattttaatctctaaaacaataactagaagccatttttattagtttgacagttaaactaaattacggttgcactattccttctattgatacgacgcaattcccatgattctaagcctggtatgtctggcttttttcgactctggttattaaaagctgttgcggactatcaaatacctcatcttttcggtccgcttctataagaatcgttttggtaaaaacctattcgaaatcaacataaacacaaaatcggtcgagccgtcacgatagaagtaatcatctctttcctaaaccttttagaccacttcatgtttctccacacgtattgttcacgatactcggtcagattctccataaaaaaagttgagctaaacggcccctaaagtatatgtagataagcacgattctagtacgactaaccgcaggacttcttgagcagagcaaaagatggatattaattacagaagaactgacggggcgcatggtttccacgaggtgggcgcaccccggccacatgcaccggcgaaaaacaaaatggcagtcgcgaatcgaaagctgccaagctaggccgcatgctggggatgtgtgacacacatatcattggcggtcatggcaggtcgacacgtgtaggcggacgtcatccatcgtcttcatccctatctatctatctatcactcagtacaagccattgctttcgttatcttccgaggcgggcagggcagggcaggttcttccccccttggcacgcacggcttgcgtggagccaggccagctcgacgatgactacgcggcccagtcctcctcttcgatcctataaaagcgaggcccagtccctccatctcaaaaatttcaatgcctctctagatcttttaagctagctatccttgcttcctcccttcactttcttattgagagctctctggcgtgctgtaccggacgacaggggttggtaggtcgtaggtgtagctagctcggcgagcgagcaatccggccaacagcacacgcgtcgatcgcc

SEQ ID NO: 25 - DNA, 1291 bp, Artificial Sequence
  ZmNac111 promoter and MITE
tctgaaagaaataaatattgctaatattttttctaaaatttagtcaaaatcaagaaagtttaaccatcatgaacatcgtagcgacactgattctaggtattaattatctgcattcaagtattcctacaggctagacgaagcaattaatatgagaaaggttgacggaggtccggggacgaaggtcataacccttgggaccattttaatctctttgtctaaaacaataactagaagccatttctattagtttgacagttaaactaaattacggttgcactattccttctattgatacgacgcaattcccatgattctaagcctggtatgtctggcttttttcgactctgggtattaaaagctgttgcggactatcaaatacctcatcttttcggtccgcttcaaaacctattcgaaatcaacataaacacaaaatcggttgagccgtcacgtcacgatagaagtaatcatctctttcctaaaccttttagaccacttcatgtttctgcacacgtattgttcacgatactcggtcagattctccataaaaaaagttgagctaaacagcccctaaagtatatgtagataagcacgattctagtactaaccgcaggacttcttgagcagagcaaaagatggatactccctccgtttctttttattagtcgctggatagtgcaattttgcactatccagcgactaataaaaagaaacggagggagtattaattacagaagaactgacggggcgcatggtttccacgaggtgggcgcaccccggccacatgcatcggcgaaaacaaaatggcagtcgcgaatcgaaagctgccaagctaggccgcatgctggggatgtgtgacacacatatcattggcggtcatggcaggtcgacacgtgtaggcggacgtcatccatcgtcttcatccctatctatctatctatcactcagtacaagccattgctttcgttatcttccgaggcgggcagggcagggcaggttcttccccccttggcacgcaccgcacgcacggcttgcgtggagccaggccagctcgacgatgactacgcggccccggccccgcggcccagtcctccctcctcttcgatcctataaaagcgaggcccagtccctccatctcaaaaatttcaatgcctctctagatctttcaagctatccttgcttcctagctccctttggcgtgctgtaccggacgacaggggtaggtaggtaggtaggtgtagctagctcggcgagcgagggagcaatccggccaacagcacacgcgtcgatcgcc

SEQ ID NO: 26 - DNA, 82 bp, Artificial Sequence
  MITE
ctccctccgtttctttttattagtcgctggatagtgcaattttgcactatccagcgactaataaaaagaaacggagggagta

SEQ ID NO: 27 - protein, 475 aa, Zea mays L.; identical to SEQ ID NO: 4

SEQ ID NO: 28 - DNA, 1824 bp, Zea mays L.; identical to SEQ ID NO: 3

SEQ ID NO: 29 - DNA, 19 bp, Artificial Sequence
  primer
atgccgagaagcggcggcg

SEQ ID NO: 30 - DNA, 20 bp, Artificial Sequence
  primer
ctactgcatcccgatgtggc

SEQ ID NO: 31 - DNA, 23 bp, Artificial Sequence
  primer
ggtaacattgtgctcagtggtgg

SEQ ID NO: 32 - DNA, 22 bp, Artificial Sequence
  primer
gcatcaattcgatcactcagag

SEQ ID NO: 33 - DNA, 20 bp, Artificial Sequence
  primer
ctactatgacgacgacaact

SEQ ID NO: 34 - DNA, 18 bp, Artificial Sequence
  primer
cactcgcttcctcttgtt

SEQ ID NO: 35 - DNA, 20 bp, Artificial Sequence
  primer
tggttgtggcttcgttggtt

SEQ ID NO: 36 - DNA, 23 bp, Artificial Sequence
  primer
gctgcagaagagttttgggtaca

SEQ ID NO: 37-38 - skipped (2 entries)

SEQ ID NO: 39 - DNA, 18 bp, Artificial Sequence
  alignment
aagatcgatattaattac

SEQ ID NO: 40 - DNA, 99 bp, Artificial Sequence
  alignment
aagatcgatactccctccgtttctttttattagtgctggatagtgcaattttgcacattccagcgactaataaaaagaaaccgaccgagtattaattac

SEQ ID NO: 41 - DNA, 84 bp, Artificial Sequence
  hairpin loop
tactccctccgtttctttttattagtcgctggatagtgcaattttgcactatccagcgactaataaaaagaaacggagggagta

SEQ ID NO: 42 - DNA, 19 bp, Artificial Sequence
  Primer
atgccgagaagcggcggcg

SEQ ID NO: 43 - DNA, 20 bp, Artificial Sequence
  Primer
ctactgcatcccgatgtggc

SEQ ID NO: 44 - DNA, 20 bp, Artificial Sequence
  Primer
aaacggctaccacatccaag

SEQ ID NO: 45 - DNA, 20 bp, Artificial Sequence
  Primer
cctccaatggatcctcgtta

SEQ ID NO: 46 - DNA, 24 bp, Artificial Sequence
  Primer
ctcaggtattgcagaccgtatgag

SEQ ID NO: 47 - DNA, 24 bp, Artificial Sequence
  Primer
ctggacctgcttcatcatactctg

SEQ ID NO: 48 - DNA, 22 bp, Artificial Sequence
  Primer
ttgtgcctccactgcaattcgg

SEQ ID NO: 49 - DNA, 26 bp, Artificial Sequence
  Primer
ttacaagtcgtctccctccatcttcc

SEQ ID NO: 50 - DNA, 20 bp, Artificial Sequence
  Primer
aatcacgggcacaaggagga

SEQ ID NO: 51 - DNA, 21 bp, Artificial Sequence
  Primer
tagacgagctggagctggaac

SEQ ID NO: 52 - DNA, 23 bp, Artificial Sequence
  Primer
tagtgtccaagccgttcctcaag

SEQ ID NO: 53 - DNA, 22 bp, Artificial Sequence
  Primer
aaggatgatggcgtggttctcg

SEQ ID NO: 54 - DNA, 24 bp, Artificial Sequence
  Primer
gtggtgggttgaggggaaggaagc

SEQ ID NO: 55 - DNA, 20 bp, Artificial Sequence
  primer
acgccatcgccgttgagcct

SEQ ID NO: 56 - DNA, 23 bp, Artificial Sequence
  primer
tcgctcacgttcgacgagttcca

SEQ ID NO: 57 - DNA, 22 bp, Artificial Sequence
  primer
tctcctcggcggtccagatgct

SEQ ID NO: 58 - DNA, 22 bp, Artificial Sequence
  primer
aaggatgcggatgatggaggac

SEQ ID NO: 59 - DNA, 24 bp, Artificial Sequence
  primer
aagtcaaagacgccgaagaagtgc

SEQ ID NO: 60 - DNA, 22 bp, Artificial Sequence
  primer
taaacgccggggacacggtagt

SEQ ID NO: 61 - DNA, 20 bp, Artificial Sequence
  primer
tttcctgtggctgtggttgg

SEQ ID NO: 62 - DNA, 18 bp, Artificial Sequence
  primer
atggacatgggccggcac

SEQ ID NO: 63 - DNA, 21 bp, Artificial Sequence
  primer
ctagtagctccagagcgcgac

SEQ ID NO: 64 - DNA, 23 bp, Artificial Sequence
  primer
agtactaaccgcaggacttctcg

SEQ ID NO: 65 - DNA, 20 bp, Artificial Sequence
  primer
gttttgttttcgccgatgca

SEQ ID NO: 66 - DNA, 26 bp, Artificial Sequence
  primer
cgatactcggtcagattctccataaa

SEQ ID NO: 67 - DNA, 23 bp, Artificial Sequence
  primer
gctcaagaagtcctgcggttagt

SEQ ID NO: 68 - DNA, 20 bp, Artificial Sequence
  primer
cgaatcgaaagctgccaagc

SEQ ID NO: 69 - DNA, 24 bp, Artificial Sequence
  primer
tagggatgaagacgatggatgacg

SEQ ID NO: 70 - DNA, 21 bp, Artificial Sequence
  primer
gcccagtccctccatctcaaa

SEQ ID NO: 71 - DNA, 20 bp, Artificial Sequence
  primer
gtccggtacagcacgccaaa

SEQ ID NO: 72 - DNA, 26 bp, Artificial Sequence
  primer
tctccctggtacgtgattctttattt

SEQ ID NO: 73 - DNA, 24 bp, Artificial Sequence
  primer
caactgggtgcagatttggattag

SEQ ID NO: 74 - DNA, 23 bp, Artificial Sequence
  primer
gtgtcttcctcctcgtcgtcctc

SEQ ID NO: 75 - DNA, 21 bp, Artificial Sequence
  primer
tgcgaaacgcttcagtcaggt

SEQ ID NO: 76 - DNA, 20 bp, Artificial Sequence
  primer
ttgccgactgctgctgctac

SEQ ID NO: 77 - DNA, 22 bp, Artificial Sequence
  primer
ccgtcactcgcttcctcttgtt

SEQ ID NO: 78 - DNA, 20 bp, Artificial Sequence
  primer
agcagcggcgtcttcttcca

SEQ ID NO: 79 - DNA, 21 bp, Artificial Sequence
  primer
tctactgcatcccgatgtggc

SEQ ID NO: 80 - DNA, 19 bp, Artificial Sequence
  primer
gatgatgcgccaagagctg

SEQ ID NO: 81 - DNA, 22 bp, Artificial Sequence
  primer
gcctcatcacctacgtaggcat

SEQ ID NO: 82 - DNA, 20 bp, Artificial Sequence
  primer
gtccggggacgaaggtcata

SEQ ID NO: 83 - DNA, 21 bp, Artificial Sequence
  primer
caggggttgcactggtagatg

SEQ ID NO: 84 - DNA, 20 bp, Artificial Sequence
  primer
ccagctcgacgatgactacg

SEQ ID NO: 85 - DNA, 22 bp, Artificial Sequence
  primer
cgacaactggaaatcgttgctc

SEQ ID NO: 86 - DNA, 23 bp, Artificial Sequence
  primer
acaaggagtggtttttcttcagc

SEQ ID NO: 87 - DNA, 20 bp, Artificial Sequence
  primer
ccatgcctcgaatcacttgg

SEQ ID NO: 88 - DNA, 21 bp, Artificial Sequence
  primer
cgatgatgatgctgagcaagt

SEQ ID NO: 89 - DNA, 19 bp, Artificial Sequence
  primer
aacgtgcatgggtcgtcat

SEQ ID NO: 90 - DNA, 28 bp, Artificial Sequence
  primer
ccgggttgagttgttatgttatgataga

SEQ ID NO: 91 - DNA, 28 bp, Artificial Sequence
  primer
ccaataatatatatatcacacatcccca

SEQ ID NO: 92 - DNA, 34 bp, Artificial Sequence
  primer
agcacgattctagtactaaccgcaggacttcttg

SEQ ID NO: 93 - DNA, 27 bp, Artificial Sequence
  primer
gactgccattttgttttcgccgatgca

SEQ ID NO: 94 - protein, 475 aa, Artificial Sequence; identical to SEQ ID NO: 4
  alignment

SEQ ID NO: 95 - protein, 391 aa, Artificial Sequence; identical to SEQ ID NO: 6
  alignment

SEQ ID NO: 96 - protein, 343 aa, Artificial Sequence; identical to SEQ ID NO: 8
  alignment

SEQ ID NO: 97 - protein, 429 aa, Artificial Sequence; identical to SEQ ID NO: 10
  alignment

SEQ ID NO: 98 - protein, 203 aa, Artificial Sequence
  alignment
FRFHPTDEELIIHYLNQKVFPSSNHHLMFSIIADVNIYKFNPWELPDKALFGENEWFFFSPRERKYPNGTRPNRAAASGYWKATGTDKPIIAKGSMQLDDWVLCRVSHKGKAPLAGGYLQGQEMAIQITIGINEYDHQLPTMDQMIGLESEELDEKVFQEGSPEYPPTPIDTSVREVLKSIERVLSVGALDELVMASSSDDAV

SEQ ID NO: 99 - protein, 325 aa, Artificial Sequence; identical to SEQ ID NO: 14
  alignment

SEQ ID NO: 100 - protein, 354 aa, Artificial Sequence; identical to SEQ ID NO: 16
  alignment

(19) United States
(12) Patent Application Publication
(10) Pub. No.: US 2019/0085355 A1
(43) Pub. Date: Mar. 21, 2019

(54) DROUGHT TOLERANT MAIZE

(71) Applicant: Institute of Botany of the Chinese Academy of Sciences, Beijing (CN)

(72) Inventors: Feng Qin, Beijing (CN); Hude Mao, Beijing (CN); Hongwei Wang, Beijing (CN)

(21) Appl. No.: 15/746,179

(22) PCT Filed: Jul. 22, 2016
(86) PCT No.: PCT/GB2016/052220

§ 371 Date: Jan. 19, 2018

(30) Foreign Application Priority Data
Jul. 23, 2015 (CN) 201510437994.8
Sep. 21, 2015 (CN) PCT/CN2015/090084

(51) Int. Cl. C12N 15/82

(52) U.S. Cl. CPC C12N 15/8273; C12N 15/8237; C12N 15/8223

(57) ABSTRACT

The present invention provides a method of producing genetically altered plants that are drought tolerant, and plants obtainable by said method and uses thereof.

### FIELD OF THE INVENTION

The invention relates to plants that are drought tolerant and related methods and uses.

### INTRODUCTION

Maize (Zea mays) production is frequently compromised by water scarcity, which is aggravated by trends of climate warming and erratic rainfall patterns on a global scale [1-2]. During the past two decades, although progress has been made towards an overall increase in maize yields, plant sensitivity to drought stress has increased [1]. It is proposed that to improve the ability of maize to overcome stress bottlenecks rather than primary productivity would be the primary driving force to produce higher yielding maize [3]. Thus, enhanced drought tolerance has become a priority trait in current maize genetic improvement efforts. However, the identification of the genetic components underlying drought tolerance has proven to be challenging. To date, no quantitative trait loci (QTLs) responsible for maize drought tolerance have been cloned, despite the reports of their mapping information [4-7].

Traditional QTL mapping, based on the genetic linkage of a certain trait with the molecular marker within a biparental segregation population, usually recombinant inbred lines (RILs), has been successfully used to identify the genes underlying QTLs for glume architecture [8], branched architecture [9], flowering time [10], photoperiod sensitivity [11], resistance to head smut [12] etc. However, it usually takes a relatively long time to generate RILs of the two parents, which are phenotypically contrasting with respect to the trait. Moreover, the mapping resolution is largely dependent on the genetic recombination among these RILs. To achieve an accurate mapping towards the gene cloning, it usually requires either a large number of RILs initially or several consecutive steps of selfing or backcrossing, in order to narrow down the region containing the candidate gene. Thus, a large number of QTL mapping studies have been conducted but only a limited number of them were cloned [13]. Another limitation of this approach is that the functional variation(s) of only two parental alleles can typically be evaluated during the mapping process.

Recently, genome-wide association study (GWAS), which is based on genetic linkage disequilibrium (LD) and makes use of natural variation and recombinants, has been used as a novel strategy for dissecting complex trait loci in plants [14-17]. Since the historical and revolutionary recombinant events can be exploited in a collection of a large number of genotypes, the LD mapping can reach a high resolution and investigate multiple alleles of a single locus [14]. With the development of high-throughput DNA variation discovery technology and improvement of statistical analyses, GWAS has gained favourability in genetic research in various plant species. Especially, due to the rapid LD decay in the maize genome, GWAS has facilitated the genetic dissection of several complex traits, including kernel β-carotene [18] and oil content [19], and flowering time in maize [20]. Although association studies of maize drought tolerance have been attempted [21-24], the proposed candidate genes or their causative variations still remain to be verified and resolved.

In the maize genome, ˜85% of the genomic contents are composed of transposable elements (TEs), and the generic sequences are embedded in a vast expanse of TEs [25]. In order to maintain stability of the genome, transposable elements (TEs) are usually silenced and inactive, due to DNA and chromatin modifications [26]. However, TEs have been shown to play important roles in plant evolution and environmental adaptation. For instance, a Hopscotch element inserted at ˜60-kb upstream of teosinte branched1 (tb1) increased maize apical dominance [9] and a CACTA-like transposable element located ˜2-kb upstream of ZmCCT was found to contribute to maize photoperiod sensitivity [20]. Miniature Inverted-repeat Transposable Elements (MITEs) are a kind of non-autonomous DNA transposon, which are usually shorter than 600 bp and widespread in plant genomes [27]. A MITE insertion in ˜70-kb upstream of ZmRAP2.7 was demonstrated to be associated with maize flowering time [10]. TEs can influence nearby gene expression either through the cis-acting element residing in their own sequences, or by changing the DNA or chromatin methylation status of adjacent genes [26, 28, 29]. In rice (Oryza sativa), MITEs have been recently discovered to be capable of generating 24-nt siRNA, depending on Dicer-like 3a (OsDCL3a) activity, and interfering with nearby gene expression through RNA-directed DNA methylation (RdDM) [30]. In plants, the RdDM pathway consists of the following major steps: (i) the RNA polymerase IV transcribes single-strand RNAs from repetitive heterochromatin regions, (ii) its physically associated RNA-dependent RNA polymerase 2 (RDR2) synthesizes the double-stranded RNA (dsRNAs), (iii) the dsRNAs are cleaved by Dicer-like 3 (DCL3) into 24-nt siRNAs, and (iv) ARGONAUTE 4 (AGO4) subsequently loads the siRNAs to their complementary DNA regions. Lastly, the formed complex recruits the DNA methyltransferase (DRM2) to catalyze methylation at cytosine in CG, CHG and CHH contents (H=A, G or C), especially in CHH sequence context, which is a hallmark of RdDM31-33. Enrichment of chromatin Histone3 lysine 9 dimethylation (H3K9me2), which is mainly catalyzed by a histone methyltransferase, SUVH4 (also named KYP), couples the DNA methylation in the adjacent regions [30, 34].

The inventors have shown by GWAS that an 82-bp (MITE) insertion in the promoter region of a NAC gene (ZmNAC111) is associated with maize drought tolerance. The MITE insertion correlates with lower ZmNac111 expression in maize, and when heterologously expressed in Arabidopsis it suppresses the ZmNac111 expression via the RdDM pathway. Transgenic studies demonstrated that enhanced expression of ZmNac111 conferred drought tolerance in both transgenic Arabidopsis and maize seedlings by improving plant water use efficiency (WUE) and enhancing the expression of stress-responsive genes under the stress. A comparison of MITE insertion frequency and nucleotide diversity at the ZmNac111 locus among teosinte, tropical/subtropical and temperate genotypes, suggests that the MITE insertion appears to have occurred after maize domestication from teosinte and spread in the temperate germplasm. The identification of this MITE insertion therefore provides an insight into the genetic natural variation of maize drought tolerance.

### SUMMARY OF THE INVENTION

The inventors have identified and characterised the ZmNac111 promoter gene in maize and have surprisingly found that in strains where a miniature inverted-repeat transposable element (MITE) is inserted into the promoter this significantly affects drought tolerance. The inventors have also generated genetically altered, specifically, transgenic maize and Arabidopsis overexpressing ZmNac111, which displayed enhanced tolerance to drought stress compared to control plants that did not overexpress ZmNac111. These plants also did not show any growth penalties. The identification of ZmNac111 and its role in conferring drought tolerance is of significant value as this makes it possible to generate drought tolerant plants, which are important in agriculture.

The invention is thus aimed at providing genetically altered plants that show drought tolerance and related methods and uses. In one embodiment, the invention is aimed at providing transgenic plants that show drought tolerance and related methods and uses.

In a first aspect, the invention relates to a genetically altered plant or part thereof expressing a nucleic acid construct comprising a nucleic acid as defined in SEQ ID NO. 1, 2 or 3 or a functional homologue or variant thereof. In one embodiment, the invention relates to a transgenic plant or part thereof expressing a nucleic acid construct comprising a nucleic acid as defined in SEQ ID NO. 1, 2 or 3 or a functional homologue or variant thereof. The invention also relates to a product derived from a plant as defined above or from a part thereof.

In another aspect, the invention relates to a vector comprising a nucleic acid as defined in SEQ ID NO. 1, 2 or 3 or a functional homologue or variant thereof.

In another aspect, the invention relates to a host cell comprising a vector as described above.

In another aspect, the invention relates to a use of a nucleic acid as defined in SEQ ID NO. 1, 2 or 3 or a functional homologue or variant thereof or a vector as described above in conferring drought tolerance.

In another aspect, the invention relates to a use of a nucleic acid as defined in SEQ ID NO. 1, 2 or 3 or a functional homologue or variant thereof or a vector as described above in increasing yield/growth of a plant under drought stress conditions.

In another aspect, the invention relates to a method for increasing drought tolerance of a plant said method comprising introducing and expressing in said plant a nucleic acid construct comprising a nucleic acid as defined in SEQ ID NO. 1, 2 or 3 or a functional homologue or variant thereof.

In another aspect, the invention relates to a method for increasing yield of a plant under drought or water deficit conditions said method comprising introducing and expressing in said plant a nucleic acid construct comprising a nucleic acid as defined in SEQ ID NO. 1, 2 or 3 or a functional homologue or variant thereof.

In a further aspect, the invention relates to a method for producing a mutant plant tolerant to drought comprising introducing a mutation into the nucleic acid sequence of endogenous ZmNAC111 or the endogenous ZmNAC111 promoter or a functional homologue or variant thereof using targeted genome modification.

In a final aspect, the invention relates to a genetically altered plant, wherein said plant carries a mutation in the endogenous NAC111 gene or NAC111 promoter gene.

The invention is further described in the following non-limiting figures.

### BRIEF DESCRIPTION OF THE FIGURES

FIG. 1 shows an 82-bp MITE insertion in the ZmNac111 promoter associated with maize drought tolerance. (a) The association analysis of genetic variation in ZmNac111 with drought tolerance in maize and the pattern of pairwise LD of DNA polymorphisms. A schematic diagram of the 2.3-kb genomic region of ZmNAC111, including the 5′-, 3′-UTR, three exons and two introns is presented. The location of the start codon (ATG) is labelled as +1′. The region encoding the NAC domain is indicated in red. The most significant InDel (InDel-572) in the promoter and two non-synonymous variations in the coding region are connected to their locations in the gene diagram by solid lines. SNP1532 produced a change of Pro to Gln in the encoded protein, and SNP1535 changed Gln to Arg. (b) The DNA sequence and structure of the 82-bp MITE inserted in the ZmNac111 promoter. The target site duplications (TSDs) and the loop are indicated by the red boxes. The blue arrows indicate two terminal inverted repeats. The predicted hairpin structure of the MITE is illustrated at the bottom. A 24-nt siRNA sequence found in the Cereal Small RNA Database that aligned to the MITE is highlighted in red. The MITE is present in the promoter of the ZmNac111 gene in many of the drought-sensitive genotypes, such as B73 and Mo17, whereas it is absent in drought-tolerant genotypes, such as CIMBL55, 92, 70 and CML118.

FIG. 2 shows the expression level of ZmNAC111. Relative expression level of ZmNac111 under well-watered, moderate and severe drought conditions in relation to the rate of plant survival, and the presence or absence of the MITE (InDel-572) insertion. (a) Correlation of plant survival rate with the relative expression level of ZmNAC111. Drought stress was estimated by the decrease in the relative leaf water content (RLWC) from 98% (well watered) to 70% (moderate drought) and to 58% (severe drought). The red and green dots represent genotypes with or without the MITE insertion (MITE+ and MITE−) in the ZmNac111 promoter, respectively. Significance is determined using the t-test. (b) Comparison of ZmNac111 expression in MITE− and MITE+ genotypes in relation to RLWC. One-way analysis of variance (ANOVA) is applied to determine statistical differences in ZmNac111 expression.

FIG. 3 shows DNA and H3K9me2 methylation status of the drought-tolerant and drought-sensitive alleles of ZmNAC111. (a) DNA methylation status was determined by treatment with McrBC, a methylation-sensitive endonuclease followed by qPCR (McrBC-qPCR) analyses in the eight regions (R1-R8) of the genomic sequence of ZmNAC111. Genomic DNA was extracted from B73 and CIMBL55 genotypes grown under well-watered (WW) and moderate drought (dry, RLWC ¼ 70%) conditions. (b) Methylation of cytosine residues in CG, CHG and CHH sites (grey, green and blue lines, respectively) was revealed by bisulphite sequencing of the BSP1 region. The DNA samples of (a) were analysed. The MITE region is indicated by a double-sided arrow. (c) Chromatin state was detected using an anti-H3K9me2 ChIP-qPCR assay at eight different regions (R1-R8) of the genomic regions of ZmNAC111-B73 and ZmNAC111-CIMBL55. The H3K9me2 state of the maize Ubi2 and Actin1 were tested in parallel as negative controls. Anti-H3 was used as an internal reference in the ChIP-qPCR assay. (d) The positions of R1-R8 in the genomic region of ZmNAC111. The 50- and 30-UTR regions (light grey boxes), exons (grey boxes) and 82-bp MITE insertion (red box) are illustrated. Black lines indicate the position of the McrBC-qPCR, ChIP-qPCR and bisulphite sequencing (BSP1) analyses. Error bars are s.d. and significant differences are determined using the t-test, *P<0.05; **P<0.01.

FIG. 4 shows the repression of ZmNac111 expression by the MITE insertion is dependent on RNA-directed DNA methylation and histone methylation when heterologously expressed. (a) Diagram of the plasmid constructs (35S:gZmNAC111-B73 and 35S:gZmNAC111-CIMBL55) that were used to transform Arabidopsis. The MITE insertion (red box), regions of the McrBC-qPCR, ChIP-qPCR (R1-R8) and bisulphite sequencing (BSP1) analyses are indicated. (b) Left panel: comparison of ZmNac111 expression in independent transgenic lines of 35S:gZmNAC111-B73 and 35S:gZmNAC111-CIMBL55. Right panel: statistical differences in the ZmNac111 gene expression in the transgenic Arabidopsis lines. (c) DNA methylation status in the eight regions (R1-R8) of the genomic sequence of 35S:gZmNAC111-B73 and 35S:gZmNAC111-CIMBL55 determined by McrBC-qPCR assays. (d) Methylation states of cytosine residues in CG, CHG and CHH sites, assayed with bisulphite sequencing of the BSP1 region. (e) Chromatin states detected at the eight regions (R1-R8), using an anti-H3K9me2 ChIP-qPCR assay. The H3K9me2 states of the 18S and Actin8 Arabidopsis genes were evaluated in parallel and served as negative controls. Anti-H3 was used as an internal reference for ChIP-qPCR. (f) qRT-PCR analysis of transcript levels of 35S:gZmNAC111-B73 in wild-type and the RdDM mutants, and 35S:gZmNAC111-CIMBL55 transcript levels in the wild type. (g) DNA methylation status determined using the McrBC-qPCR assay of the R1 region in the designated genetic backgrounds. (h) Chromatin states detected using anti-H3K9me2 ChIP-qPCR assays at R1 (left column) and R2 (right column) region. ‘ x dcl2-1;dcl4-2’; ‘x suvh4-3’; ‘x rdr2-2’; ‘x ago4-5’; and ‘ x drm1-2;drm2-2’ in (f) and (g) indicate the homozygous genetic background of the 35S:gZmNAC111-B73-2 (left column) and -23 (right column) transgenics after crossing. Green columns indicate that ZmNac111 expression (f), DNA methylation (g) and H3K9me2 (h) were comparable with levels in the 35S:gZmNAC111-CIMBL55 transgenics in the wild-type background, whereas red columns indicate that they are significantly different with those in the 35S:gZmNAC111-CIMBL55 transgenics. (i) Methylation of cytosine residues assayed with bisulphite sequencing of the BSP1 region of the 35S:gZmNAC111-B73 transgenics in the different RdDM-defective mutant backgrounds. The MITE region is indicated by double-sided arrows. Error bars are s.d. and significant differences are determined using the t-test, *P<0.05; **P<0.01.

FIG. 5 shows drought tolerance of ZmUbi:ZmNAC111 transgenic maize. (a) The growth phenotype of the T1 generation transgenic and sibling transformation-negative (WT) plants. Four representative independent transgene-positive lines (ZmNAC111-OE1, OE3, OE4 and OE7) and the WT are shown. (b) Transcript levels of ZmNac111 in the WT, and three independent ZmUbi:ZmNAC111 transgenic maize lines. (c) Drought tolerance of T2 seedlings of ZmUbi:ZmNAC111 transgenic maize compared with WT. Photographs were taken under well-watered conditions and subsequent to a drought treatment followed by re-watering for a period of 7 days. The survival rates of WT and transgenic ZmUbi:ZmNAC111-OE1, OE3 and OE7 plants were compared. (d) Statistical analysis of survival rates after drought treatment and recovery. The average percentage of survival and standard errors were calculated from four independent experiments. (e-h) Comparison of the photosynthetic performance of ZmUbi:ZmNAC111 transgenic and WT plants during the process of the drought stress. (e) Photosynthesis rate; (f) stomatal conductance; (g) transpiration rate; and (h) water-use efficiency. Error bars are s.d. and significant differences are determined using the t-test, *P<0.05; **P<0.01.

FIG. 6 shows a transcriptome analysis and frequency of MITE insertion. Transcriptomic analysis of ZmUbi:ZmNAC111 transgenic maize, and comparison of MITE insertion frequency and nucleotide diversity of ZmNac111 in teosinte, TST and temperate maize inbred lines. (a) Venn diagrams of upregulated or downregulated genes in ZmUbi:ZmNAC111-OE1 and OE3 plants relative to WT plants using a significance cutoff of P<0.001, and a fold change (FC)>2. (b) Hierarchical clustering of differentially expressed genes in the transgenic lines relative to WT plants. The indicated scale is the log 2 value of the normalized level of gene expression. (c) Gene ontology of biological pathways enriched in the transgenic lines based on the upregulated or downregulated genes. Significant differences are determined using the t-test, *P<0.05; **P<0.01. (d) qRT-PCR verification of increased expression of genes involved in plant drought response and tolerance in the transgenics under normal and drought conditions. Error bars are s.d. (e) Frequency of MITE insertion in the ZmNac111 promoter in teosinte, TST and temperate (including non-stiff stalk (NSS), stiff stalk (SS) and mixed) 51 maize inbred lines. (f) Nucleotide diversity at the ZmNac111 locus among teosinte and MITE (maize) and MITE [symbol] (maize [symbol] ) inbred lines of maize. Nucleotide diversity was compared across the ZmNac111 locus, among the 72 MITE [symbol] and 190 MITE− maize inbred lines and 42 teosinte entries. ‘P’ denotes the ZmNac111 promoter region. Nucleotide diversity (it) for teosinte (itT, grey), the maize inbred lines of maize (it [symbol] , red) and Maize (it_, green) was calculated using a 100-bp sliding window and 25-bp step. The Tajima's D values of different regions are shown. *P<0.05; **P<0.01; ***P<0.001.

FIG. 7 shows a genome-wide association study analysis, which reveals that a SNP located in GRMZM2G127379 was significantly associated with plant drought tolerance in maize. GRMZM2G127379, ZmNAC111, is indicated in red. A 0.5 Mb region of chromosome 10 is displayed. The physical position of the predicted genes is based on the MaizeGDB release 5b.60. The association of each marker with drought tolerance was calculated using Tassel 3.1.0, under the standard mixed linear model (MLM, MAF 0.05).

FIG. 8 shows a phylogenetic tree of stress-related NAC proteins in maize, rice, sorghum and Arabidopsis. A Neighbor-joining phylogenetic tree was constructed based on the sequence alignments of 55 full-length NAC-domain-containing proteins from four species. Gene codes and names are illustrated in red for maize; blue for rice; black for sorghum; and green for Arabidopsis. The bar indicates the relative divergence of the sequences examined and bootstrap values from 1,000 replicates were displayed next to the branch.

FIG. 9 shows the phenotype of six maize inbred lines. (a) Survival rate of B73, Mo17, CML118, CIMBL70, CIMBL92 and CIMBL55 plants subjected to severe drought stress. (b) Expression levels of ZmNac111 in B73, Mo17, CML118, CIMBL70, CIMBL92 and CIMBL55 under well-watered, moderate, and severe drought conditions. The level of drought severity was assessed as a decrease in RLWC from 98% (well-watered) to 70% (moderate drought), to 58% (severe drought). Error bars are s.d.

FIG. 10 shows the drought-tolerant allele of ZmNac111 co-segregates with drought tolerance in three F2:3 populations of maize. (a) Survival rate of CIMBL55, CIMBL91, CIMBL9, GEMS54 and BY4944 plants subjected to severe drought stress. (b) Expression levels of ZmNac111 in CIMBL55, CIMBL91, CIMBL9, GEMS54 and BY4944 under well-watered, moderate, and severe drought conditions. The level of drought severity was assessed as a decrease in RLWC from 98% (well-watered) to 70% (moderate drought), to 58% (severe drought). (c) A representative photograph of the genotyping of F2 individuals based on the 82-bp MITE insertion in the three segregating populations. P1 and P2 represent the two parents of the corresponding population. The size of the DNA band from CIMBL9, GEMS54 and BY4944 was 206-bp; and the band from CIMBL55, CIMBL91 was 124-bp in length. (d) The number of F2 individual plants segregating for the MITE insertion: homozygous MITE−/− (tolerant allele), homozygous of MITE+/+(sensitive allele), and heterozygous MITE−/+. (e) The effect of the ZmNac111 tolerant allele on drought tolerance in three F2:3 segregating populations. The survival rates of the F3 lines carrying either the homozygous tolerant or sensitive allele of ZmNac111 were compared in the three populations. Error bars are s.d. and significant differences were determined using a t-test, *P<0.05, **P<0.01.

FIG. 11 shows the transactivation activity of different ZmNac111 proteins encoded by the genotypes with the two non-synonymous variations. (a) The name of different maize inbred lines and their genotypes at the two significant non-synonymous sites in the coding region. (b) The yeast strain AH109 transformed with a vector (pGBKT7) carrying the ZmNac111 gene, cloned from CIMBL19, 123, 22, 91, 55, B73, Mo17, D863F, BY4944, and Shen5003 inbred lines. Cultures of transformed yeast cells were diluted and placed on agar culture plates containing a-tryptophan (-T), synthetic dropout (SD) medium (SD/-T), a-tryptophan-histidine (SD/-T-H) medium, or a-tryptophan-histidine-adenine (SD/-T-H-A) medium. The photographs were taken of 3-day-old cultures on the corresponding medium.

FIG. 12 shows siRNAs aligned to the 82-bp MITE insertion in the ZmNAC111-B73 allele. (a) The structure diagram of the ZmNAC111-B73 allele. The exons are in black boxes and the MITE insertions are in a red box. (b) The siRNAs profiles of the ZmNAC111-B73 allele. Tracks indicate the position of the aligned, unique siRNAs obtained from the Cereal Small RNA Database. The lengths of the mapped small-RNAs are denoted by different colours.

FIG. 13 shows drought tolerance of 35S:gZmNAC111-B73 and 35S:gZmNAC111-CIMBL55 transgenic Arabidopsis. (a) Drought tolerance of 35S:gZmNAC111-B73 and 35S:gZmNAC111-CIMBL55 transgenic Arabidopsis. Photographs were taken before and after the drought treatment followed by a six-day period of re-watering. Vector-transformed Arabidopsis (VC) and 35S:gZmNAC111-B73-2, -23 and 35S:gZmNAC111-CIMBL55-5, -12 transgenic plants are shown. (b) qRT-PCR analysis of ZmNac111 transcript level in the four independent lines. (c) Statistical analysis of survival rates after the drought-stress treatment. The average survival rate and standard error were calculated based on data obtained from three independent experiments. Error bars are s.d. and significant differences were determined using a t-test, *P<0.05, **P<0.01.

FIG. 14 shows DNA methylation and Histone H3K9me2 of 35S:gZmNAC111-CIMBL55 in the RdDM mutants. (a) qRT-PCR analysis of transcript levels of 35S:gZmNAC111-B73 in wild-type and 35S:gZmNAC111-CIMBL55 in wild-type and the RdDM mutant background. (b) DNA methylation status of the R1 region were determined by the McrBC-qPCR assay in the designated genetic backgrounds. “xsuvh4-3”; “xrdr2-2”; and “xago4-5” in (a) and (b) indicate the homozygous genetic background of the 35S:gZmNAC111-CIMBL5-5 (left column) and -12 (right column) transgenics after crossing. (c) Chromatin states detected by anti-H3K9me2 ChIP-qPCR assays at R1 (left column) and R2 (right column) region. The ChIP assay was performed using two independent F3 homozygous lines in each of the designated genetic backgrounds. Green columns indicate that ZmNac111 expression (a), DNA methylation (b), and H3K9me2 (c) were comparable with levels in the 35S:gZmNAC111-CIMBL55 transgenics in the wild-type background; whereas red columns indicate that they were significantly different with those in the 35S:gZmNAC111-CIMBL55 transgenics. (d) Methylation of cytosine residues assayed with bisulfite sequencing of the BSP1 region of the 35S:gZmNAC111-CIMBL55 transgenics in the different RdDM mutant backgrounds. Error bars are s.d. and significant differences were determined using a t-test, *P<0.05, **P<0.01.

FIG. 15 shows the phenotype of the 35S:ZmNAC111 transgenic Arabidopsis. (a) Drought tolerance of transgenic Arabidopsis plants overexpressing ZmNAC111. Photographs were taken before and after the drought treatment followed by a six-day period of re-watering. Vector-transformed Arabidopsis (VC) and ZmNAC111-OE6, OE7 and OE8 transgenic plants are shown. (b) qRT-PCR analysis of ZmNac111 transcript levels in the three independent lines. (c) Statistical analysis of survival rates after the drought-stress treatment. The average survival rates and standard errors were calculated based on data obtained from three independent experiments. (d) Effect of exogenous ABA on seed germination. Seeds of VC and ZmNAC111-OE6, OE7 and OE8 transgenic plants were placed on half-strength MS plates supplemented with 0.5 μM and 1 μM ABA and germination was scored by the appearance of radicals. Plant images were obtained 7-day after placing seeds on the MS plates. (e) ABA-induced stomatal closure in VC and ZmNAC111-OE6, OE7 and OE8 transgenic plants. Epidermal peels were used to measure the size of stomatal apertures in response to ABA at 0.1, 1.0, and 10 μM. (f) Statistical analysis of (d) was based on data obtained from three independent experiments using 100 seeds in each experiment. (g) Statistic analysis of (e) was based on data obtained from three replicates and the presented values represent the means±s.d. (n=45). Error bars are s.d. and significant differences were determined using a t-test, *P<0.05, **P<0.01.

FIG. 16 shows the transcriptomic analysis of ZmUbi:ZmNAC111 transgenic maize under well-watered conditions. (a) Venn diagrams of ZmNac111 up-regulated and down-regulated genes (P<0.001, FC>2.0) in two ZmUbi:ZmNAC111-OE1 and ZmUbi:ZmNAC111-OE3 transgenic maize in relation to WT plants (OE1 and OE3). (b) Hierarchical clustering of ZmNac111 up-regulated and down-regulated genes in OE1 and OE3 plants. The scale represents the log 2 value of the normalized level of gene expression. (c) Enriched GOBPs based on up- and down-regulated genes (P<0.01, P-value was computed by DAVID, indicating the significant of the enrichment) in ZmNac111 transgenic plants.

FIG. 17 shows a phenotypic comparison between ZmUbi:ZmNAC111 transgenic maize and sibling transformation-negative (WT) plants in T2 generations under well-watered conditions. N, number of plants; PH, plant height; EH, ear height; NN, node numbers; LN, leaf numbers; LEA, leaf numbers above the ear; TL, tassel length; LW, leaf width of the top ear; LL, leaf length of the top ear. Data are shown as mean±s.d.

FIG. 18 shows the primers used herein. The name of the primers was based on the gene name and experimental purpose. Numbers in the brackets indicate the location of the primer within the corresponding gene. The location of the start codon (ATG) was considered as +1.

FIG. 19 shows an alignment of the ZmNAC11 homologs.

FIG. 20 is an RNA-seq analysis of ZmNAC111 transgenic Arabidopsis under normal growing conditions.

FIG. 21 is an RNA-seq analysis of ZmNAC111 transgenic Arabidopsis under normal drought treatment.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA. The terms “peptide”, “polypeptide” and “protein” are used interchangeably herein and refer to amino acids in a polymeric form of any length, linked together by peptide bonds.

As used herein, the term “genetically altered” includes, but is not limited to, transgenic plants and mutant plants.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or

(b) genetic control sequence(s) which is operably linked with the nucleic acid sequence according to the invention, for example a promoter, or

(c) a) and b)

are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 both incorporated by reference.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, breeding methods, stable transformation methods, transient transformation methods, and virus-mediated methods. Methods are known in the art for the targeted insertion of a polynucleotide at a specific location in the plant genome.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. According to the invention, the transgene is stably integrated into the plant and the plant is preferably homozygous for the transgene. Thus, any off spring or harvestable material derived from said plant is also preferably homozygous for the transgene.

The aspects of the invention involve recombination DNA technology and in a preferred embodiment exclude embodiments that are solely based on generating plants by traditional breeding methods.

For the purposes of the invention, a “mutant” plant is a plant that has been genetically altered compared to the naturally occurring wild type (WT) plant. In one embodiment, a mutant plant is a plant that has been altered compared to the naturally occurring wild type (WT) plant using a mutagenesis method, such as the mutagenesis methods described herein. In one embodiment, the mutagenesis method is targeted genome modification or genome editing. In one embodiment, the endogenous ZmNac111 promoter sequences in wheat have been altered compared to wild type sequences using a mutagenesis method. These mutations may cause activation or otherwise enhance the activity of the ZmNac111 promoter or a functional homologue or variant thereof. Such plants have an altered phenotype and show tolerance or increased tolerance to drought compared to wild type plants. Therefore, the tolerance is conferred by the presence of a mutated endogenous ZmNac111 promoter gene in the wheat plant genome. In a preferred embodiment, the endogenous ZmNac111 promoter sequence is specifically targeted using targeted genome modification and is not conferred by the presence of transgenes expressed in wheat. In an alternative embodiment, there is provided a mutant plant that expresses a nucleic acid as defined in SEQ ID NO: 1, 2 or 3, or a functional homologue or variant thereof. Again, such plants have an altered phenotype and show tolerance or increased tolerance to drought compared to wild type plants. Also, again, in one embodiment, the phenotype of such plants is not conferred by the presence of a transgene(s).

The inventors have identified that a miniature inverted-repeat transposable element (MITE) that is inserted into the promoter of a NAC gene (ZmNAC111) significantly affects drought tolerance and that furthermore, overexpression of ZmNac111 in transgenic maize enhances drought tolerance.

A control plant as used herein is a plant which has not been modified according to the methods of the invention. Accordingly, the control plant has not been genetically modified or altered to express a nucleic acid as described herein. In one embodiment, the control plant is a wild type plant. In another embodiment, the control plant is a plant that does not carry a transgene according to the methods described herein, but expresses a different transgene. In another embodiment, the control plant is plant that has not been subjected to targeted genome modification or editing. The control plant is typically of the same plant species, preferably the same ecotype as the plant to be assessed.

Thus, in a first aspect, the invention relates to a genetically altered plant expressing a nucleic acid construct comprising a ZmNac111 nucleic acid sequence or a variant or homologue thereof. In one embodiment, the invention relates to a transgenic plant expressing a nucleic acid construct comprising a ZmNac111 nucleic acid sequence or a variant or homologue thereof Thus, the genetically altered, or in one embodiment, transgenic plant includes within its genome a nucleic acid construct comprising a ZmNac111 nucleic acid sequence. In one embodiment, wherein the plant is a transgenic plant, preferably, said plant is homozygous for the presence of the transgene.

In one embodiment, the ZmNac111 nucleic acid sequence comprises or consists of SEQ ID NO: 1, 2 or 3 or a functional homologue or variant thereof. SEQ ID NO: 1 represents the genomic DNA. Residues 157 to 364, 534 to 872 and 968 to 1850 of SEQ ID NO: 1 are the coding regions (SEQ ID NO: 2). SEQ ID NO: 1 is the nucleotide sequence of ZmNac111 of the inbred maize line B73. The accession number is GRMZM2G127379. In a further embodiment, the ZmNac111 nucleic acid sequence comprises or consists of SEQ ID NO: 3 or a functional homologue or variant thereof. SEQ ID NO: 3 is the cDNA sequence of ZmNAC111.

The polypeptide encoded by SEQ ID NO: 1, 2, 3 or a functional homologue or variant thereof comprises or consists of SEQ ID NO: 4 or a functional homologue or variant thereof. Thus, the genetically altered, or, in one embodiment, transgenic plant of the invention expresses a ZmNac111 nucleic acid sequence and produces a protein that comprises or consists of SEQ ID NO: 4 or a functional homologue or variant thereof.

According to the various aspects of the invention, the term “functional homologue or variant of a nucleic acid sequence” as used herein, for example with reference to SEQ ID NO: 1, 2, 3 or 4 or homologs thereof, refers to a variant gene sequence or part of the gene sequence which retains the biological function of the full non-variant ZmNac111 gene or ZmNac111 protein sequence, for example confers drought tolerance when expressed in a non-genetically altered or transgenic plant. A functional variant also comprises a variant of the gene of interest encoding a polypeptide which has sequence alterations that do not affect function of the resulting protein, for example in non-conserved residues. Also encompassed is a variant that is substantially identical, i.e. has only some sequence variations, for example in non-conserved residues, to the wild type sequences as shown herein and is biologically active.

Thus, it is understood, as those skilled in the art will appreciate, that the aspects of the invention, including the methods and uses, encompass not only a ZmNac111 nucleic acid or ZmNac111 protein sequence as described herein, for example a nucleic acid sequence comprising or consisting or SEQ ID NO: 1, 2, 3, a polypeptide comprising or consisting or SEQ ID NO: 4, but also functional homologues or variants of a ZmNac111 gene or ZmNac111 protein that do not affect the biological activity and function of the resulting protein. Alterations in a nucleic acid sequence which result in the production of a different amino acid at a given site that do however not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

Generally, variants of ZmNac111 have at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the sequences represented by SEQ ID NO: 1, 2, 3 or 4.

A biologically active variant of a ZmNac111 protein may differ from that protein by as few as 1-15 amino acid residues, as few as 1-10, such as 6-10, as few as 5, as few as 4, 3, 2, or even 1 amino acid residue. In certain embodiments, ZmNac111 proteins may be altered in various ways including amino acid substitutions, deletions, truncations, and insertions. Methods for such manipulations are generally known in the art. For example, amino acid sequence variants and fragments of the ZmNac111 protein can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. The deletions, insertions, and substitutions of the protein sequences encompassed herein are not expected to produce radical changes in the characteristics of the protein. When it is difficult, however, to predict the exact effect of a substitution, deletion, or insertion in advance of making such modifications, one skilled in the art will appreciate that the effect will be evaluated by routine screening assays.

The term homologue as used herein also designates a NAC111 orthologue from other plant species. A homologue of ZmNac111 polypeptide has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by SEQ ID NO: 4. Preferably, overall sequence identity is more than 70% or more than 73%. Preferably, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%. In another embodiment, the homologue of a NAC111 nucleic acid sequence has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid represented by SEQ ID NO: 1, 2 or 3. Preferably, overall sequence identity is more than 70% or more than 73%. Preferably, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%. The overall sequence identity is determined using a global alignment algorithm known in the art, such as the Needleman Wunsch algorithm in the program GAP (GCG Wisconsin Package, Accelrys). Variants of homologs are also within the scope of the invention.

For example, sequence identity/similarity values provided herein can refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percentage sequence identity may be adjusted upwards to correct for the conservative nature of the substitution. Sequences that differ by such conservative substitutions are said to have “sequence similarity” or “similarity”.

In one embodiment of the plants, methods and uses described herein, the functional homologue is NAC10, for example OsNac10 (see SEQ ID NO: 5 and 6).

In one embodiment of the plants, methods and uses described herein, the functional homologue is as shown in SEQ ID No 5 or 6, 7 or 8, 9 or 10, 11 or 12, 13 or 14, 15 or 16 or a variant thereof.

In one embodiment, the ZmNAC111amino acid sequence encoded by SEQ ID NO: 1, 2 or 3 or a functional homologue or variant thereof (SEQ ID NO: 4) is characterised by the presence of a conserved motif. In a functional ZmNac111 variant protein, changes to the amino acid sequence are preferably located outside these domains.

In one embodiment, a ZmNac111 homologue comprises a NAC DNA-binding domain. NAC DNA-binding domains are typically around 160 amino acids and are divided into five sub-domains, classed A, B, C, D and E and, in one embodiment, have the following consensus sequence:

sub-domain A:
(SEQ ID NO: 17)
LPPGFRFHPTDEELICHYL
sub-domain B:
(SEQ ID NO: 18)
IIAEVDLYKCEPWDLPEKCKI
sub-domain C:
(SEQ ID NO: 19)
WYFFCPRDRKYPNGTRTNRATGSGYWKATGKDKEI
sub-domain D:
(SEQ ID NO: 20)
VGMRKTLVFYMGRAPRGTKTNWVMHEFRL
sub-domain E:
(SEQ ID NO: 21)
DEWVVCKVHHK

or variants thereof. A variant is as defined herein.

In one embodiment, a NAC111 homologue comprises a NAC DNA-binding domain having the following sequence:

(SEQ ID NO: 22)
LPAGFRFHPTDEELMVHYLMRQAASMPCPVPIIAEVNIYQCNPWDLPAKA
LFGDKEWFFFSPRDRKYPNGARPNRAAGSGYWKATGTDKAILSSSTPTSH
GGANIVVGVKKALVFYGGRPPKGTKTDWIMHEYRLSGAADDDCKGSTRRR
VSSSSSSSMRLDDWVLCRIHKK

or a variant thereof. In one embodiment, the domain has at least 80%, for example 90%, 91%, 92%, 93%, 94%, 95%, 96%, 97%, 98%, or at least 99% sequence identity to this domain.

In a further embodiment, a ZmNac111 homologue comprises a NAC DNA-binding domain, as defined above (SEQ ID NO: 22) and/or is a functional homologue, meaning the homologue retains the biological function of the ZmNac111 gene or ZmNac111 protein sequence, for example confers drought tolerance when expressed in a genetically altered or, in one embodiment, transgenic plant and/or has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by SEQ ID NO: 4 or to the nucleic acid represented by SEQ ID NO: 1, 2 or 3.

Suitable homologues can be identified by sequence comparisons and identifications of conserved domains. There are predictors in the art that can be used to identify such sequences. The function of the homologue can be identified as described herein and a skilled person would thus be able to confirm the function when expressed in a plant. Thus, one of skill in the art will recognize that analogous amino acid substitutions listed above with reference to SEQ ID NO: 4 can be made in ZmNac111 from other plants by aligning the polypeptide sequence to be mutated with the ZmNac111 polypeptide sequence as set forth in SEQ ID NO: 4.

Thus, the nucleotide sequences of the invention and described herein can be used to isolate corresponding sequences from other organisms, particularly other plants, for example crop plants. In this manner, methods such as PCR, hybridization, and the like can be used to identify such sequences based on their sequence homology to the sequences described herein. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

Preferred homologues of ZmNac111 peptides are ZmNac111 homologues from crop plants, for example cereal crops. In one embodiment, preferred homologues include maize, rice, wheat, sorghum, sugar cane, oilseed rape (canola), soybean, cotton, potato, tomato, tobacco, grape, barley, pea, bean, field bean or other legumes, lettuce, sunflower, alfalfa, sugar beet, broccoli or other vegetable brassicas or poplar. Preferred homologues and their peptide sequences are also shown in SEQ ID Nos 5 to 16.

Also, the various aspects of the invention the aspects of the invention, including the methods and uses, encompass not only a ZmNac111 nucleic acid sequence as shown herein, but also a fragment thereof. By “fragment” is intended a portion of the nucleotide sequence or a portion of the amino acid sequence and hence of the protein encoded thereby. Fragments of a nucleotide sequence may encode protein fragments that retain the biological activity of the native protein and hence confer drought tolerance.

In one embodiment according to the various aspects of the invention, the nucleic acid construct comprises a regulatory sequence or element. According to the various aspects of the invention, the term “regulatory element” is used interchangeably herein with “control sequence” and “promoter” and all terms are to be taken in a broad context to refer to regulatory nucleic acid sequences capable of effecting expression of the sequences to which they are ligated. The term “regulatory element” also includes terminator sequences which may be included 3′ of the ZmNac111 nucleic acid sequence. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern. For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes are known to the skilled person and include for example beta-glucuronidase or beta-galactosidase.

In one embodiment, the ZmNac111 nucleic acid is operably linked to a regulatory sequence or element. The term “operably linked” as used herein refers to a functional linkage between the promoter sequence and the gene of interest, such that the promoter sequence is able to initiate transcription of the gene of interest.

In one preferred embodiment, the nucleic acid sequence may be expressed using a promoter that drives overexpression. Overexpression according to the invention means that the transgene is expressed at a level that is higher than expression of endogenous counterparts driven by their endogenous promoters. For example, overexpression may be carried out using a strong promoter, such as a constitutive promoter. A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, maize ubiquitin promoter, rubisco small subunit, maize or alfalfa H3 histone, OCS, SAD1 or 2, GOS2 or any promoter that gives enhanced expression. Alternatively, enhanced or increased expression can be achieved by using transcription or translation enhancers or activators and may incorporate enhancers into the gene to further increase expression. Furthermore, an inducible expression system may be used, where expression is driven by a promoter induced by environmental stress conditions, in particular drought. The promoter may also be tissue-specific. The types of promoters listed above are described in the art. Other suitable promoters and inducible systems are also known to the skilled person.

In a one embodiment, the promoter is a constitutive or strong promoter. In one embodiment, the promoter is Zmubi1. In one embodiment, the promoter has the sequence of SEQ ID NO: 23, or a variant as defined herein.

In another embodiment the regulatory sequence is the CaMV35S promoter.

In one embodiment, the promoter is a ZmNac111 promoter isolated from a drought tolerant maize inbred line. Such promoter does not contain a polymorphism at the following position: InDel-572 with respect to SEQ ID NO: 2 (the A in the ATG site is designated as +1; this is the first residue in SEQ ID NO: 2) compared to a drought sensitive line. In particular, this promoter can be used to express ZmNac111 at the onset of drought stress. In one embodiment, the ZmNac111 promoter or variant thereof, has the sequence SEQ ID NO: 24

Additional nucleic acid sequences which facilitate cloning of the target nucleic acid sequences into an expression vector may also be included in the nucleic acid construct according to the various aspects of the invention. This encompasses the alteration of certain codons to introduce specific restriction sites that facilitate cloning. A terminator sequence may also be included in the construct.

In one embodiment, the plant is maize and the nucleic acid construct comprising ZmNac111 may be expressed in a maize plant by recombinant methods. In another embodiment, an exogenous ZmNac111 nucleic acid is expressed in a second plant of another species by recombinant methods. Thus, all aspects of the invention, including the genetically altered, e.g. transgenic plants and methods of the invention, also extend to plants other than maize which express a nucleic acid construct comprising a ZmNac111 nucleic acid sequence.

In one embodiment, the plant is a monocot or dicot plant. In one embodiment, the plant is a crop plant or biofuel plant.

In one embodiment of the various aspects of the invention, the plant is a dicot plant. A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as rape/canola, corn, sugar cane, palm trees, jatropha, soybeans, sorghum, sunflowers, cottonseed, Panicum virgatum (switchgrass), linseed, wheat, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

In one embodiment of the various aspects of the invention, the plant is a dicot plant. A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

In preferred embodiments of the various aspects of the invention the plant is a crop plant. By crop plant is meant any plant which is grown on a commercial scale for human or animal consumption or use.

In preferred embodiments of the various aspects of the invention the plant is selected from a grain plant, an oil-seed plant, and a leguminous plant.

Most preferred plants according to the various aspects of the invention are maize, rice, wheat, oilseed rape, sorghum, soybean, potato, tomato, tobacco, grape, barley, pea, bean, field bean, lettuce, cotton, sugar cane, sugar beet, broccoli or other vegetable brassicas or poplar.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

The term “maize” as used herein refers to a plant of the Zea mays L. ssp. mays and is also known as “corn”. The term “maize plant” includes: whole maize plants, maize germplasm, maize plant cells, maize plant protoplast, maize plant cell or maize tissue cultures from which maize plants can be regenerated, maize plant calli, and maize plant cells that are intact in maize plants or parts of maize plants, such as maize seeds, maize cobs, maize flowers, maize cotyledons, maize leaves, maize stems, maize buds, maize roots, maize root tips, and the like. The maize can be an inbred line, or a maize hybrid such as a maize single cross hybrid.

The various aspects of the invention described herein clearly extend to any plant cell or any plant produced, obtained or obtainable by any of the methods described herein, and to all plant parts and propagules thereof unless otherwise specified. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also extends to harvestable parts of a plant of the invention as described above such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins. The invention also relates to products, including food products and food supplements comprising the plant of the invention or parts thereof.

The plant according to the invention shows increased resistance or tolerance to drought or water deficiency compared to a control plant.

In one embodiment, said stress is moderate or severe stress. A plant according to the invention also shows reduced growth/yield penalties under moderate stress compared to a control plant.

In one embodiment, the methods of the invention thus relate to increasing resistance or tolerance to moderate (non-lethal) stress or severe stress. In the former embodiment, genetically altered, or in one embodiment, transgenic plants according to the invention show increased resistance or tolerance to stress and therefore, the plant yield is not or less affected by the stress compared to wild type yields which are reduced upon exposure to stress. In other words, an improvement in yield under moderate stress conditions can be observed.

For example, drought tolerance is assessed predominantly under quite severe conditions in which plant survival is scored after a prolonged period of soil drying. However, in temperate climates, limited water availability rarely causes plant death, but restricts biomass and seed yield. Moderate water stress, that is suboptimal availability of water for growth can occur during intermittent intervals of days or weeks between irrigation events and may limit leaf growth, light interception, photosynthesis and hence yield potential. Leaf growth inhibition by water stress is particularly undesirable during early establishment. There is a need for methods for making plants with increased yield under moderate stress conditions. In other words, whilst plant research in making stress tolerant plants is often directed at identifying plants that show increased stress tolerance under severe conditions that will lead to death of a wild type plant, these plants do not perform well under moderate stress conditions and often show growth reduction which leads to unnecessary yield loss. Thus, in one embodiment of the methods of the invention, yield is improved under moderate stress conditions. The genetically altered, such as transgenic plants according to the various aspects of the invention show enhanced tolerance to these types of stresses compared to a control plant and are able to mitigate any loss in yield/growth. The tolerance can therefore be measured as an increase in yield as shown in the examples. The terms moderate or mild stress/stress conditions are used interchangeably and refer to non-severe stress. In other words, moderate stress, unlike severe stress, does not lead to plant death. Under moderate, that is non-lethal, stress conditions, wild type plants are able to survive, but show a decrease in growth and seed production and prolonged moderate stress can also result in developmental arrest. The decrease can be at least 5%-50% or more. Tolerance to severe stress is measured as a percentage of survival, whereas moderate stress does not affect survival, but growth rates. The precise conditions that define moderate stress vary from plant to plant and also between climate zones, but ultimately, these moderate conditions do not cause the plant to die. Importantly, the Inventors have identified that there are no growth penalties observed in the genetically altered plants described herein.

Generally speaking, moderate drought stress is defined by a water potential of between −1 and −2 Mpa.

In one embodiment, the maize relative leaf water content (RLWC) at 95-100% is well-watered or favourable growth condition; RLWC at around 70-65% is moderate drought stress; RLWC at around 58-55% is severe drought stress.

Drought tolerance can be measured using methods known in the art, for example assessing survival of the genetically altered plant compared to a control plant, through leaf water potentials or by determining turgor pressure, rosette radius, water loss in leaves, growth or yield. Drought tolerance can also be measured by assessing stomatal conductance (Gst) and transpiration in whole plants under basal conditions.

According to the invention, a genetically altered plant, such as, in one embodiment, a transgenic plant has enhanced drought tolerance if the survival rates are at least 2, 3, 4, 5, 6, 7, 8, 9 or 10-fold higher than those of the control plant after exposure to drought and/or after exposure to drought and re-watering. Also according to the invention, a genetically altered plant, such as a transgenic plant has enhanced drought tolerance if the rosette radius is at least 10, 20, 30, 40, 50% larger than that of the control plant after exposure to drought and/or after exposure to drought and re-watering. The plant may be deprived of water for 10-30, for example 20 days and then re-watered. Also according to the invention, a genetically altered plant, such as a transgenic plant has enhanced drought tolerance if stomatal conductance (Gst) and transpiration are lower than in the control plant, for example at least 10, 20, 30, 40, 50% lower.

The terms “increase”, “improve” or “enhance” are interchangeable. Yield for example is increased by at least a 3%, 4%, 5%, 6%, 7%, 8%, 9% or 10%, preferably at least 15% or 20%, more preferably 25%, 30%, 35%, 40% or 50% or more in comparison to a control plant. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. Thus, according to the invention, yield comprises one or more of and can be measured by assessing one or more of: increased seed yield per plant, increased seed filling rate, increased number of filled seeds, increased harvest index, increased number of seed capsules/pods, increased seed size, increased growth or increased branching, for example inflorescences with more branches. Preferably, yield comprises an increased number of seed capsules/pods and/or increased branching. Yield is increased relative to control plants.

In another aspect, the invention relates to an isolated nucleic acid comprising or consisting of SEQ ID NO: 1, 2 or 3 or a functional homologue or variant thereof. In another aspect, the invention relates to an isolated amino acid sequence comprising or consisting of SEQ ID NO: 4 or a functional homologue or variant thereof.

In another aspect, the invention relates to a vector comprising a nucleic acid construct comprising SEQ ID NO: 1, 2 or 3 or a functional homologue or variant thereof. In one embodiment, said vector is an expression vector. Expression vectors for expressing nucleic acid sequences in a plant are well known. An example is pGXX. For example, a ZmNac111 nucleic acid sequence as described herein can be inserted between the Smal and Sall restriction sites of the pGXX vector. Plant expression vectors also include dual agrobacterium vectors and plant micro bombardment vectors such as pROKII, pBin438, pCAMBIA1302, pCAMBIA2301, pCAMBIA1301, pCAMBIA1300, pBI121, pSBII, pCAMBIA1391-Xa or pCAMBIA1391-Xb.

The vector may further comprise a regulatory sequence which directs expression of the nucleic acid. Such sequences are described elsewhere herein. In one example, the regulatory sequence is a promoter that directs overexpression of the nucleic acid sequence. Marker genes (e.g Gus) and resistance genes can also be included.

In another aspect, the invention relates to a host cell comprising a vector as described herein. The host cell can be selected from a plant cell or a bacterial cell, for example Agrobacterium. The invention also relates to a culture medium or kit comprising a culture medium and an isolated host cell as described above.

In another aspect, the invention relates to the use of a nucleic acid construct comprising or consisting of SEQ ID NO: 1, 2 or 3 or a functional homologue or variant thereof or a vector described herein in conferring drought tolerance to a plant.

In another aspect, the invention relates to the use of a nucleic acid construct comprising or consisting SEQ ID NO: 1, 2 or 3 or a functional homologue or variant thereof or a vector described herein in increasing yield/growth of a plant under drought stress conditions.

In another aspect, the invention relates to a method for conferring to or increasing drought tolerance of a plant said method comprising introducing and expressing in said plant a nucleic acid construct comprising or consisting of SEQ ID NO: 1, 2 or 3 or a functional homologue or variant thereof. In another aspect, the invention relates to a method for increasing yield of a plant, for example under moderate drought stress, said method comprising introducing and expressing in said plant a nucleic acid construct comprising or consisting of SEQ ID NO: 1, 2 or 3 or a functional homologue or variant thereof.

The term plant is defined elsewhere herein.

In one embodiment, said construct further comprises a regulatory sequence. Such sequences are described elsewhere herein. In one example, the regulatory sequence is a promoter that directs overexpression of the nucleic acid sequence.

The nucleic acid or vector described above is used to generate genetically altered, and in one embodiment, transgenic plants using transformation methods known in the art. Thus, according to the various aspects of the invention, a nucleic acid comprising a ZmNac111 nucleic acid or a functional homologue or variant thereof is introduced into a plant and expressed as a transgene. The nucleic acid sequence is introduced into said plant through a process called transformation. The term “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art. The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker such as the ones described above. Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The invention relates to a method for producing a genetically altered plant with improved drought tolerance compared to a control plant comprising
a) introducing into said plant and expressing a nucleic acid construct comprising a ZmNac111 nucleic acid sequence, for example a nucleic acid sequence comprising SEQ ID NO: 1, 2, 3 a functional homologue or variant of SEQ ID NO: 1, 2 or 3 and
b) obtaining a progeny plant derived from the plant or plant cell of step a).
In one embodiment, the method is a method for producing a transgenic plant.

Thus, the invention relates to a method for producing a genetically altered plant with improved yield under water deficiency or drought stress comprising
a) introducing into said plant and expressing a nucleic acid construct comprising a ZmNac111 nucleic acid sequence, for example a nucleic acid sequence comprising SEQ ID NO: 1, 2 or 3, a functional homologue or variant of SEQ ID NO: 1, 2 or 3 and
b) obtaining a progeny plant derived from the plant or plant cell of step a).
Again, in one embodiment, the method is a method for producing a transgenic plant.

In one embodiment, the drought stress is moderate.

The methods above may comprise the further steps of:
detecting the presence of the genetic alteration or transgene by methods known in the art;
exposing the plant to stress conditions, such as drought;
assessing yield/growth;
selecting a plant or part thereof with increased stress resistance/improved yield/growth;
optionally harvesting parts of the plant.

The invention also relates to plants obtained or obtainable with said method. The term plant is defined elsewhere herein.

The invention also relates to a plant with increased expression of an endogenous nucleic acid as defined in SEQ ID NO: 1, 2 or 3 or a functional homologue or variant thereof wherein said endogenous promoter (SEQ ID NO: 24) carries a mutation introduced by mutagenesis or genome editing which results in increased expression of the nucleic acid as defined in SEQ ID NO: 1, 2 or 3 or a functional homologue or variant thereof.

The invention also relates to a method for increasing expression of a nucleic acid as defined in SEQ ID NO: 1, 2 or 3 or a functional homologue or variant thereof in a plant, producing plants, a method for mitigating the impacts of stress conditions on plant growth and yield and a method for producing plants with improved yield/growth under stress conditions comprising the steps of mutagenising a plant population, identifying and selecting plants with an improved yield/growth under stress conditions and identifying a variant ZmNac111 promoter sequence which directs expression of a nucleic acid as defined in SEQ ID NO: 1, 2 or 3 or a functional homologue or variant thereof. The above can be achieved using targeted genome editing.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customizable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate its nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of Fokl to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the Fokl nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Reference 71 describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct.

Another genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

Cas9 is thus the hallmark protein of the type II CRISPR-Cas system, and a large monomeric DNA nuclease guided to a DNA target sequence adjacent to the PAM (protospacer adjacent motif) sequence motif by a complex of two noncoding RNAs: CRIPSR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3.

Cas9 expression plasmids for use in the methods of the invention can be constructed as described in the art.

Thus, aspects of the invention involve targeted mutagenesis methods, specifically genome editing, and in a preferred embodiment exclude embodiments that are solely based on generating plants by traditional breeding methods

Accordingly in one aspect of the invention there is provided a method for producing a mutant plant resistant to drought comprising introducing a mutation into the nucleic acid sequence of the endogenous ZmNac111 promoter or a functional homologue or variant thereof using targeted genome modification. In a preferred embodiment, the mutation is introduced using ZFNs, TALENs or CRISPR/Cas9.

In one embodiment, the ZmNac111 promoter or a functional homologue or variant thereof is isolated from a drought-resistant maize inbred line. In an alternative embodiment, the ZmNac111 promoter or a functional homologue or variant thereof is isolated from a drought-sensitive maize inbred line.

In one embodiment, the ZmNac111 promoter is represented by SEQ ID NO: 24

In an alternative embodiment, the ZmNac111 promoter is represented by SEQ ID NO: 25 (this sequence includes the MITE).

In one embodiment targeted genome editing or modification as defined above is used to delete at least one residue from the nucleic acid sequence of the ZmNac111 promoter, or a functional homologue or variant thereof. In a preferred embodiment, the targeted genome editing is used to delete the following sequence from a ZmNac111 promoter containing the below sequence (for example, SEQ ID NO: 26), or a functional homologue or variant thereof

In another embodiment, targeted genome editing is used to insert at least one nucleic acid in the nucleic acid sequence of the ZmNac111 promoter, or a functional homologue or variant thereof. In a preferred embodiment, this mutation enhances activity of the endogenous promoter. For example, targeted genome modification can be used to insert at least one enhancer or promoter site, such as a TATA box (TATAAA), a GC box (GGGCGG) or a CAAT (GGCCAATCT) box, or functional variants thereof.

The invention also relates to plants obtained or obtainable with said method. The term plant is defined elsewhere herein. There is also provided the use of a mutated endogenous ZmNac111 promoter as described above or a functional homologue or variant thereof to increase yield and/or growth of a plant under drought stress conditions. Alternatively, there is also provided the use of a mutated endogenous ZmNac111 promoter as described above or a functional homologue or variant thereof to confer drought tolerance.

In a final aspect of the invention there is provided a genetically altered plant expressing a nucleic acid construct comprising a nucleic acid as defined in SEQ ID NO: 1, 2 or 3 or a functional homologue or variant thereof, wherein the nucleic acid further comprises at least one of the following mutations: SNP1532: C/A and/or SNP1535:A/G. In one embodiment, the plant is a mutant plant. There is also provided a method for producing a genetically altered plant resistant to drought comprising introducing a mutation into the nucleic acid sequence of SEQ ID NO: 1, 2 or 3, wherein said mutation is SNP1532:C/A and/or SNP1535:A/G. Again, in a preferred embodiment, the plant is a mutant plant.

In all aspects of the invention where we discuss conferring drought tolerance, the invention can equally apply to conferring drought resistance.

### Further Embodiments of the Invention

A further objective of the present invention is to provide a plant drought tolerant related protein ZmNAC111, an encoding gene thereof and an application thereof.

The protein provided in this invention is derived from corn (Zea mays L.) and has a name of ZmNAC111. Said protein is a protein of a) or b):

a) a protein consisting of an amino acid sequence shown in SEQ ID NO. 27 in the sequence list;

b) a protein derived from the protein a), wherein one or more amino acid residues in the amino acid sequence shown in SEQ ID NO. 27 is substituted, removed and/or added, and the protein relates to drought tolerance.

The amino acid shown in SEQ ID NO. 27 consists of 475 amino acid residues. In order to make the protein in (a) easier for purification, a tag shown in Table 1 can be applied to connect to the amino terminal or carboxyl terminal of the protein consisting of the amino acid sequence shown in SEQ ID NO. 27.

| Tag | Residue(s) | Sequence |
|---|---|---|
| Poly-Arg | 5-6 (normally 5) | RRRRR |
| Poly-His | 2-10 (normally 6) | HHHHHH |
| FLAG | 8 | DYKDDDDK |
| Strep-tag II | 8 | WSHPQFEK |
| c-myc | 10 | EQKLISEEDL |

The protein in (b) can be obtained by artificial synthesis or obtained by synthesizing the encoding gene first and then expressing the gene biologically. The encoding gene of protein (b) can be obtained by deleting one or more codons of amino acid residues, carrying out a missense mutation of one or more bases, and/or adding a tag as shown in Table 1 to the 5′ terminal and/or 3′ terminal, of the DNA sequence shown by locus 157-1584 of SEQ ID No.28.

Encoding the above-mentioned DNA molecule of the protein also falls into the protection scope of the invention.

Said DNA molecule is a DNA molecule of 1), 2), 3) or 4):
1) a DNA molecule including a coding region as shown in SEQ ID NO: 28;
2) a DNA molecule including a coding region as shown in locus 157-1584 of SEQ ID NO. 28;
3) a DNA molecule having at least 70%, at least 75%, at least 80%, at least 85%, at least 90%, at least 95%, at least 96%, at least 97%, at least 98% or at least 99% homology with the DNA sequences defined in 1) or 2) and encoding the protein described herein;
4) a DNA molecule hybridizing with a DNA sequence defined in 1), 2) or 3) under strict conditions and encoding the above-mentioned proteins.

SEQ ID No. 28 consists of 1824 deoxynucleotides and it is the whole length cDNA sequence encoding said protein, wherein locus 157-1584 is the coding region.

The above-mentioned strict condition can be as follows: performing hybridization in a mixed solution of 7% lauryl sodium sulfate (SDS), 0.5M Na3PO4 and 1 mM EDTA at 50° C., and washing in 2×SSC and 0.1% SDS at 50° C.; or performing hybridization in a mixed solution of 7% SDS, 0.5M Na3PO4 and 1 mM EDTA at 50° C., and washing in 1×SSC and 0.1% SDS at 50° C.; or performing hybridization in a mixed solution of 7% SDS, 0.5M Na3PO4 and 1 mM EDTA at 50° C., and washing in 0.5×SSC and 0.1% SDS at 50° C.; or performing hybridization in a mixed solution of 7% SDS, 0.5M Na3PO4 and 1 mM EDTA at 50° C., and washing in 0.1×SSC and 0.1% SDS at 50° C.; or performing hybridization in a mixed solution of 7% SDS, 0.5M Na3PO4 and 1 mM EDTA at 50° C., and washing in 0.1×SSC and 0.1% SDS at 65° C.; or performing hybridization in a mixed solution of 6×SSC, 0.5% SDS at 65° C. and washing the membrane in 2×SSC and 0.1% SDS and 1% SDS respectively.

Recombinant vectors, expression cassettes, transgenic cell lines, recombinant bacteria or recombinant viruses containing the above-mentioned DNA molecules also fall into the protection scope of the present invention.

Recombinant expression vectors containing said genes can be constructed with the existing plant expression vectors. Said plant expression vectors contain dual agrobacterium vectors and vectors that can be used for plant bombardment and so on. Examples are pROKII, pBin438, pCAMBIA1302, pCAMBIA 2301, pCAMBIA 1301, pCAMBIA 1300, pBI121, pCAMBIA 1391-Xa or pCAMBIA 1391-Xb (CAMBIA Cor.) and so on. Said plant expression vectors may also contain non-translational domains in the 3′ terminal of foreign genes, namely contain a polyadenylation signal or any other DNA fragments involving in the process of mRNA modification or gene expression. Said polyadenylation signal can direct a polyadenylic acid into the 3′ terminal of an mRNA precursor, for example non-translational domains of 3′ terminal transcriptions such as agrobacterium crown gall inducible (Ti) plasmid genes (such as nopaline synthase Nos genes), and plant genes (such as soybean storage protein genes) all possess the similar functions.

When using said genes to construct the recombinant plant expression vectors, any kinds of enhancing type of promoters (such as cauliflower mosaic virus (CAMV) 35S promoters, corn ubiquitin promoters (Ubiquitin)), constitutive promoters or tissue specific expression promoters (such as seed specific expression promoters) can be added before the initial nucleotide transcription, all of which can be used separately or in combination with other promoters. Besides, when using the genes in this application to construct the plant expression vectors, an enhancer including a translational enhancer or a transcription enhancer can also be used. These enhancer domains can be ATG initial promoters or initial promoters in the adjacent domain, but they must be the same with the reading frame of the encoding sequences so as to ensure the correct translation of the entire sequence. Said translational control signals and initial promoters have a wide source and they can be obtained from natural source or can be artificially synthesized. The translational initial domain can originate from the transcription domain or structural genes.

In order to identify or select the transgenic plant cells or plants, plant expression vectors can be modified, for example, by adding genes that can express in plants (GUS gene, luciferase gens and so on) and encode enzymes capable of producing colour changes or luminous compounds; tag genes for antibiotics (such as nptll genes that give resistance to kanamycin and related antibiotics, bar genes that give resistance to herbicide phosphinothricin, hph genes that give resistance to antibiotic hygromycin and dhfr genes that give resistance to methatrexate as well as EPSPS genes that give resistance to glyphosates) or tag genes for anti-chemical agents (such as anti-herbicide genes); as well as mannose-phoshpate isomerase genes providing the metabolic capability for mannose.

In one embodiment, the recombinant vectors are pGZ or pSBIII.

The recombinant vector pGZ is the DNA fragment (downstream of 35S promoter) between Not I and Xho I cleavage sites of ZmNAC111 replacement vector pGKX shown by locus 157-1584 in SEQ ID NO. 28, while other sequences on the vector remain unchanged.

The recombinant vector pSBIII is the DNA fragment (downstream of Zmubil promoter) between Sma I and Hind III cleavge sites of ZmNAC111 replacement vector pSB II shown by locus 157-1584 in SEQ ID NO. 28, while other sequences on the vector remain unchanged.

The use of the above-mentioned proteins, DNA molecules or recombinant vectors, expression cassettes, transgenic cell lines, recombinant bacteria or recombinant viruses in modulating the adversity resistance of a plant is also within the ambit of the present invention.

In said use, modulating the stress resistance of a plant is to improve the stress resistance of the plant, and said stress resistance is drought tolerance. In one embodiment, the plant is a monocotyledon or dicotyledon.

The use of the above-mentioned proteins, DNA molecules or recombinant vectors, expression cassettes, transgenic cell lines, recombinant bacteria or recombinant viruses in cultivating a transgenic plant having stress resistance is also within the ambit of the present invention. Again, preferably, said stress resistance is drought tolerance, and preferably, said plant is a monocotyledon or dicotyledon.

Another objective of the present invention is to provide a method of cultivating a transgenic plant having a stress resistance, comprising the step of: introducing the above-described DNA molecule into a target plant to obtain a transgenic plant; the transgenic plant has a higher stress resistance than the target plant. In the above method, the stress resistance is drought tolerance and said plant is a monocotyledon or dicotyledon.

In the above method, said DNA molecule is introduced into the target plant through a recombinant vector pGZ or pSBIII.

Preferably, in the above method or use, said dicotyledon can specifically be Arabidopsis thaliana and said monocotyledon can specifically be corn (Zea mays L.).

The drought tolerance can be expressed with the following properties:
1) under drought stress, the survival rate of the transgenic plant is higher than that of the target plant;
2) under ABA stress, the germination of the transgenic plant seed is later than that of the target plant;
3) under ABA stress, the degree and rate of stomatal closure of the transgenic plant is higher than that of the target plant;
4) under drought stress, the net photosynthetic rate, stomatal conductance and/or transpiration rate is lower than that of the target plant.

Accordingly, the invention can be described in the following aspects:
1. A protein, that is a protein of a) or b):
   a) a protein consisting of an amino acid sequence shown in SEQ ID NO. 27; or
   b) a protein derived from the protein a), wherein one or more amino acid residues in the amino acid sequence shown in SEQ ID NO. 27 is substituted, removed and/or added, and the protein relates to drought tolerance.
2. A DNA molecule encoding the protein according to aspect 1.
3. The DNA molecule according to aspect 2 characterised in that the DNA molecule is a DNA molecule of 1), 2), 3) or 4):
   1) a DNA molecule including a coding region as shown in SEQ ID No. 28 in the sequence list;
   2) a DNA molecule including a coding region as shown in locus 157-1584 of SEQ ID No. 28 in the sequence list;
   3) a DNA molecule having at least 70%, at least 75%, at least 80%, at least 85%, at least 90%, at least 95%, at least 96%, at least 97%, at least 98% or at least 99% homology with DNA sequences defined in 1) or 2) and encoding the protein in aspect 1;
   4) a DNA molecule hybridizing with a DNA sequence defined in 1), 2) or 3) under strict conditions and encoding the above-mentioned proteins.
4. A recombinant vector, an expression cassette, a transgenic cell line, a recombinant bacteria or a recombinant virus comprising the DNA molecule according to aspects 2 or 3.
5. Use of the protein according to aspect 1, or the DNA molecule according to aspects 2 or 3, or the recombinant vector, expression cassette, transgenic cell line, recombinant bacteria or recombinant virus according to aspect 4 in modulating stress resistance of a plant.
6. The use according to aspect 5, characterised in that the stress resistance is drought tolerance, and preferably the plant is a monocotyledon or a dicotyledon.
7. Use of the protein according to aspect 1, or the DNA molecule according to aspect 2 or 3, or the recombinant vector, expression cassette, transgenic cell line, recombinant bacteria or recombinant virus according to aspect 4 in cultivating a transgenic plant having stress resistance.
8. The use according to aspect 7, characteristic in that the stress resistance is drought tolerance, and preferably the plant is a monocotyledon or a dicotyledon.
9. A method of cultivating a transgenic plant with stress resistance comprising: introducing the DNA molecule according to aspect 2 or 3 into a target plant to obtain a transgenic plant; the transgenic plant has a higher stress resistance than the target plant.
10. The use according to aspect 9, wherein the stress resistance is drought tolerance, and preferably, the plant is a monocotyledon or a dicotyledon.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described. The invention is further described in the following non-limiting examples.

### Example 1: ZmNac111 Associated with Drought Tolerance of Maize Seedlings

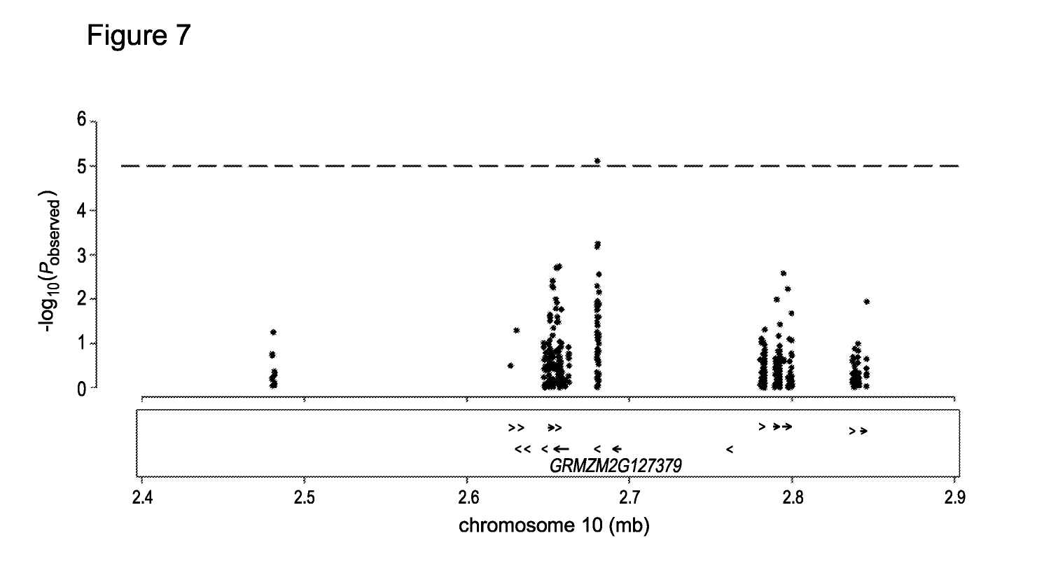

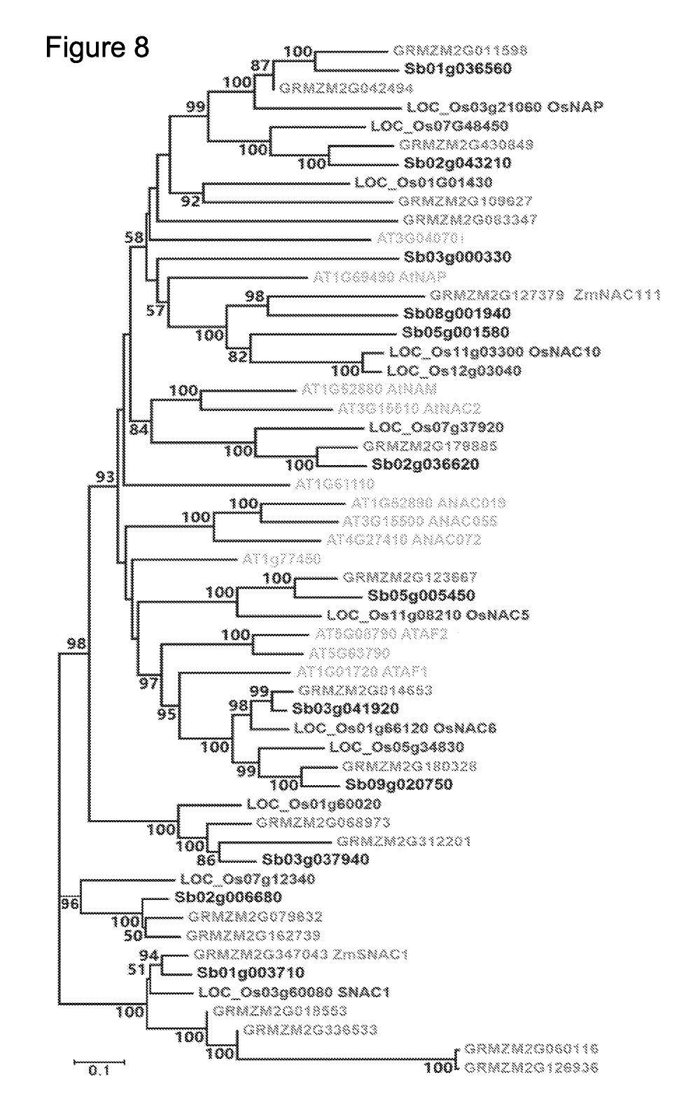

To identify genes associated with maize drought tolerance, we performed GWAS by analyzing a natural maize population, consisting of 368 inbred lines that were collected from tropical/subtropical (TST) and temperate regions of the world [19]. Approximately, 560,000 single nucleotide polymorphisms (SNPs)[19] were applied to the study. Considering the complexity of plant drought tolerance, which is affected by both the time period and intensity of the stress imposed to plants, we decided to focus on tolerance to severe drought stress at the seedling stage. Seedling survival rate (SR) can reflect plant tolerance mechanisms and cellular responses to drought. It is less affected by environmental fluctuation, which helps to identify the underlying genetic determinant(s). The drought tolerance of each genotype was assayed by calculating a SR index (percentage of the survived plants after re-watering) under severe drought stress at seedling stage [24]. On average, the inbred lines from TST exhibited higher SR in comparison with those from temperate regions, indicating that maize germplasm derived from areas near to the place of origin may be more drought tolerant than those cultivated in temperate regions. On the whole genome scale, in consideration of the population structure (Q) and parental relatedness (K) of the population, a SNP within GRMZM2G127379 on chromosome 10 was identified to be significantly associated with plant drought tolerance (FIG. 7). GRMZM2G127379 encodes a NAC-type transcription factor (TF), belonging to a family with more than 100 members in maize genome, and previously GRMZM2G127379 has been designated as ZmNAC11135. Phylogenetic analysis of the amino acid sequence encoded by ZmNac111 indicated that its closest identified homologous gene in rice is OsNAC10 (LOC_Os11 g03300)36 (FIG. 8).

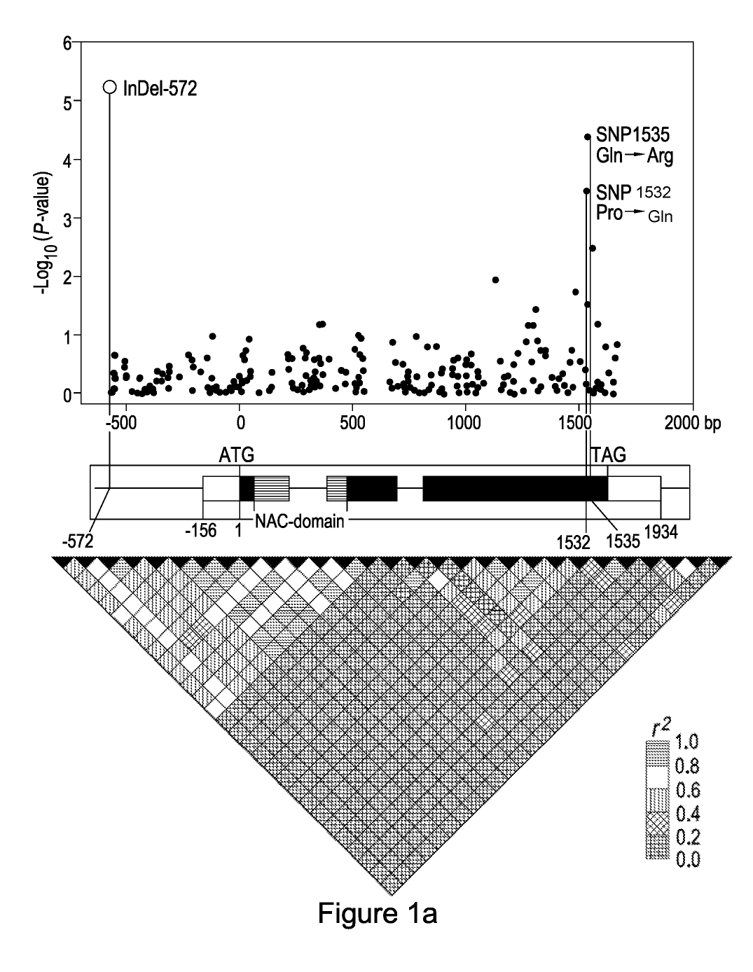

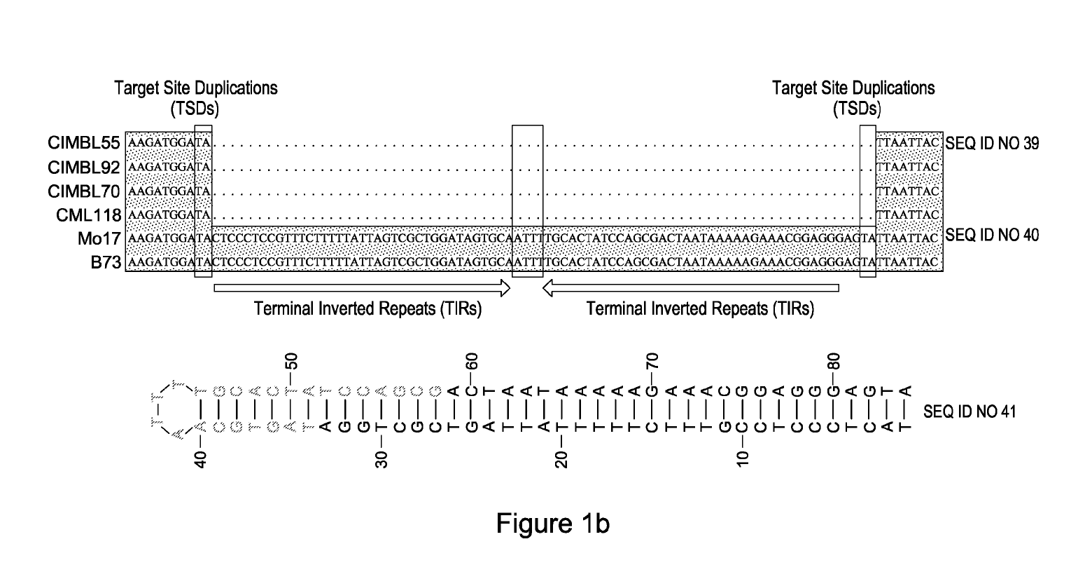

NAC proteins regulate multiple biological processes in plants, including cotyledon [37] and root development [38], formation of secondary walls [39], leaf senescence [40], nutrient remobilization to grains [41], and stress responses [42]. Considering the possible function of NAC-type genes in plant drought tolerance, we then sequenced the ZmNac111 gene in 262 maize inbred lines. A 2.3-kb genomic region, spanning the 5′ to 3′-untranslation region (UTR) of ZmNAC111, was analyzed. A total of 157 SNPs and 119 InDels (Insertions and Deletions) were further identified. A newly identified 82-bp InDel (InDel-572), located 572-bp upstream of the start codon of ZmNAC111, was found to have the greatest significant association with the seedling SR (P=5.52×10-6, calculated under mixed linear model, see Methods, FIG. 1a), contributing to 7.27% of the phenotypic variation in the natural population. Two nonsynonymous variations in exon3 were also identified as marginally significant. SNP1532 resulted in an amino acid residue change of proline (Pro) into glutamine (Gin), and SNP1535 resulted in an alteration of Gln to arginine (Arg). Both mutations, which were in strong LD, locate in the C-terminal transcriptional regulatory region but not in the N-terminal DNA-binding domain of the ZmNac111 protein. InDel-572 was in LD with the variations in 5′-UTR and the first exon (r2>0.4), but not with the two nonsynonymous SNPs (FIG. 1a). All the other variations were not associated to the trait with statistical significance.

### Example 2: InDel-572 is an 82-Bp MITE Insertion in the Promoter of ZmNac111 Gene

Sequence analysis of InDel-572 in the promoter region of ZmNac111 gene revealed that it is composed of a long terminal inverted repeat (TIR) (38-bp for each), a 4-bp loop and two additional nucleotides “TA” at the end. Another “TA” sequence was found directly prior to this insertion (FIG. 1b). It represents a typical structure of a MITE insertion within the genome, which is usually short with approximately hundreds of bps and consists of TIRs and target-site direct repeats, with a preferential insertion at TA or TAA27. We blasted the maize TE database using the 82-bp sequence as a query (http://maizetedb.org/˜maize/) and found that it belongs to the Tc1/Mariner superfamily of MITEs. The 80-bp DNA sequence, excluding the target-site direct repeat, can form a perfect stem-loop structure (FIG. 1b). It is present in the promoter of ZmNac111 of drought-sensitive genotypes, such as B73 and Mo17, whereas it is absent in the drought-tolerant genotypes, such as CIMBL55, 92, 70 and CML118 (FIG. 1b).

### Example 3: The MITE Insertion Represses ZmNac111 Expression

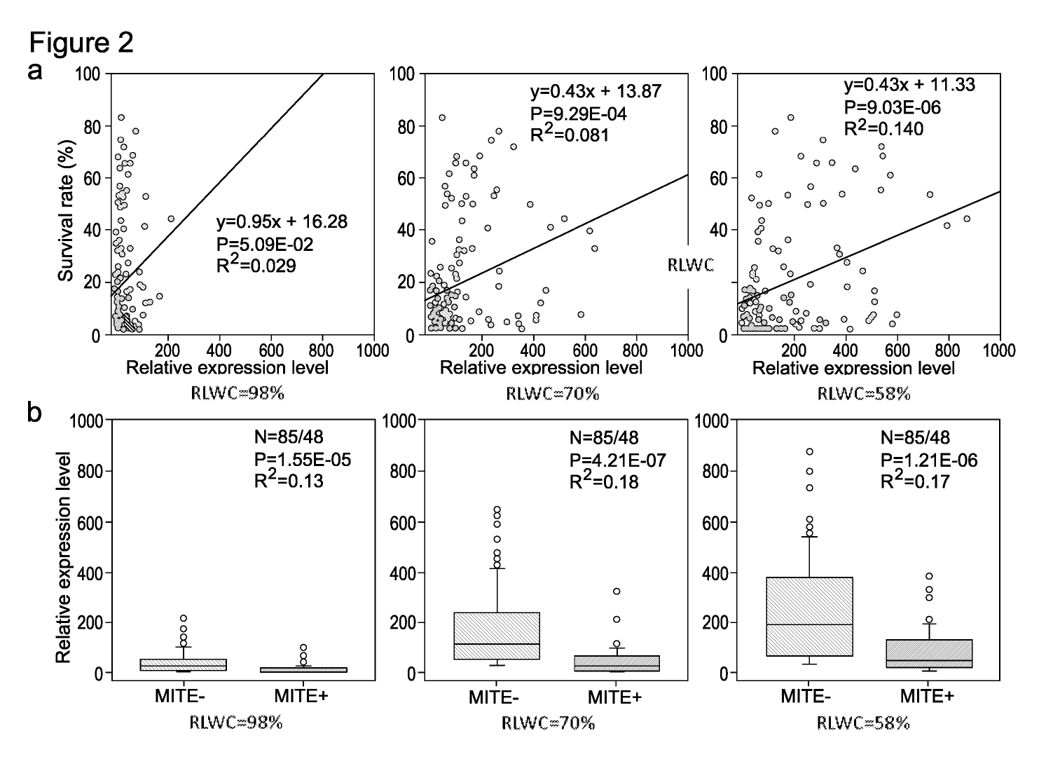

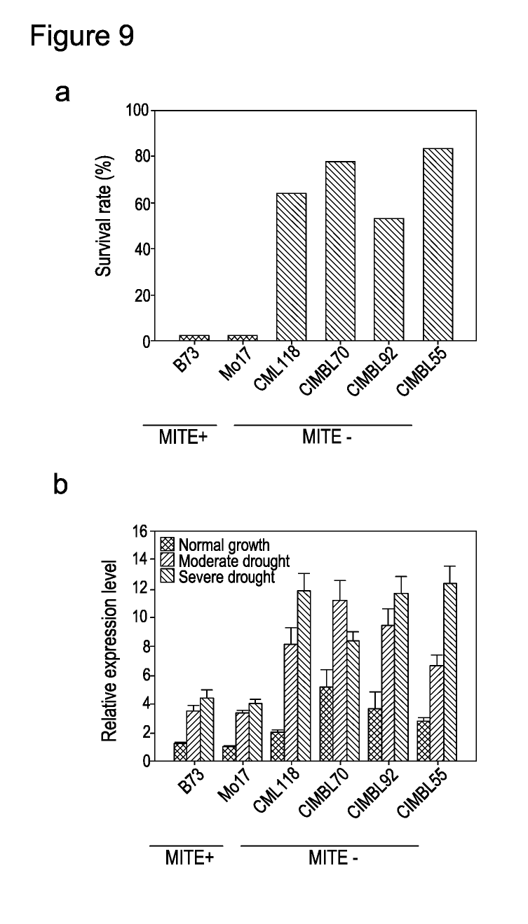

Since the MITE insertion locates in the promoter region of ZmNAC111, we hypothesized that it results in altered ZmNac111 expression among different genotypes. To examine this hypothesis, we analyzed the expression of the ZmNac111 gene among 133 inbred lines under well-watered, moderate, and severe drought conditions. Quantitative RT-PCR (qRT-PCR) analysis of a total of 399 RNA samples revealed that ZmNac111 expression positively correlated with plant survival rate under both moderate and severe drought stresses, but not under well-watered conditions (FIG. 2a). This finding suggested that increased expression of ZmNac111 might contribute to the drought tolerance of natural maize varieties examined in this study under water stress. Furthermore, the qRT-PCR result revealed that regardless of the levels of stress imposed, the genotypes without the MITE (MITE−) had significantly higher expression of ZmNac111 than those with the MITE insertion (MITE+) (FIG. 2b). The contrasting phenotype of drought tolerance and the differential ZmNac111 expression of several representative genotypes were shown (FIG. 9). On the basis of this data, we suggest that the MITE insertion may repress ZmNac111 expression, resulting in higher sensitivity of MITE+ maize varieties to drought stress.

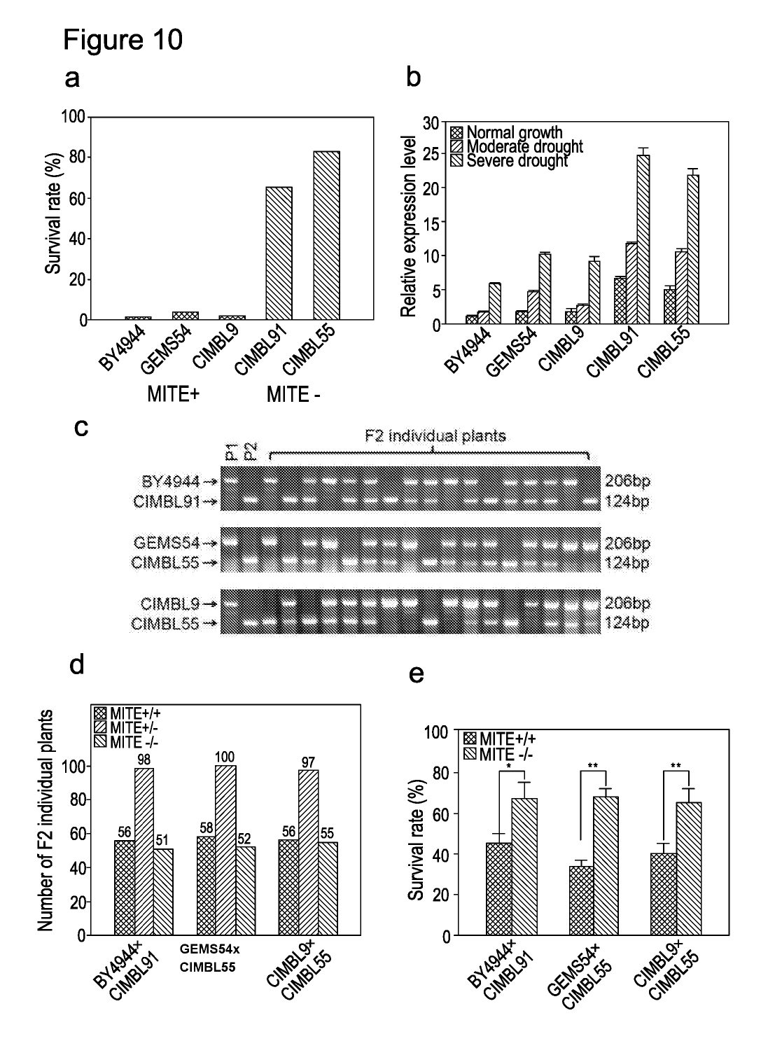

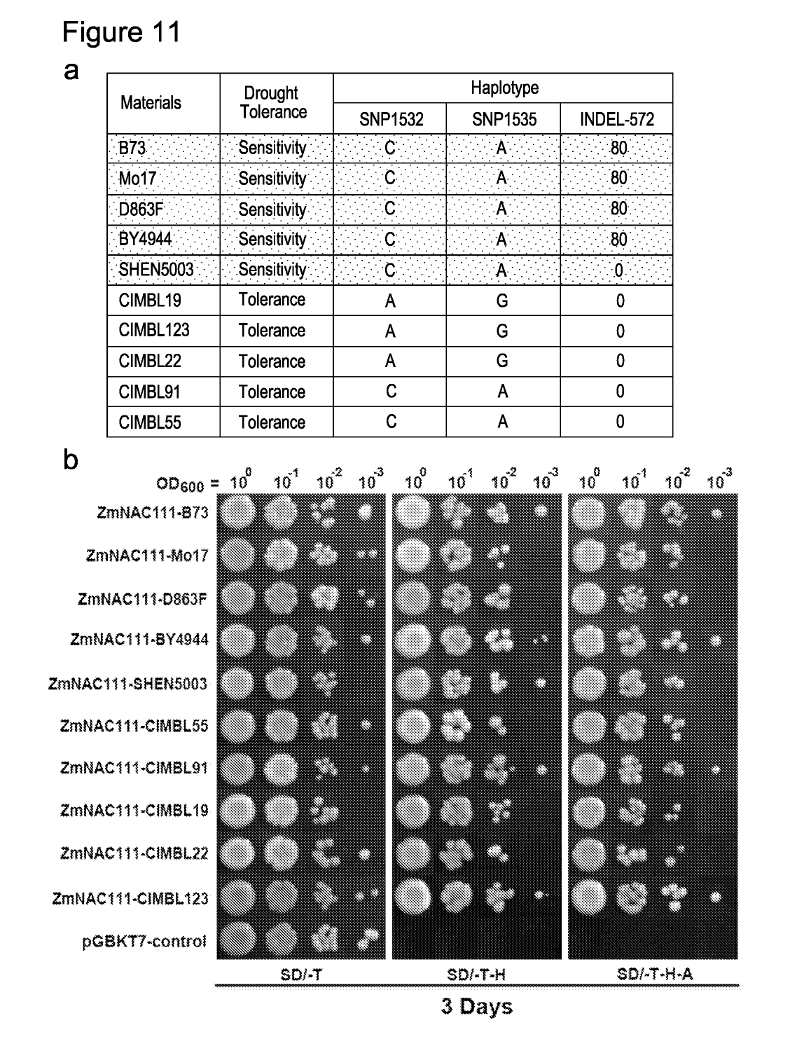

To further test whether the MITE insertion is the causative variant at the ZmNac111 locus, we constructed three bi-parental F2:3 populations. The genotypes of two parents of each population were either MITE+ or MITE− at the ZmNac111 locus. We observed that the MITE− allele co-segregated with drought tolerance in three bi-parental F2:3 populations (FIG. 10). On the other hand, when we compared the transactivation activity of ZmNac111 proteins encoded by alleles that differed in the two nonsynonymous variations, SNP1532 and SNP1535, we found that these two variations in different alleles did not significantly affect the transactivation activity of the ZmNac111 (FIG. 11). Collectively, on the basis of our results, we suggest that the MITE insertion is the likely cause for differences in drought tolerance associated with ZmNAC111, rather than amino acid changes in the encoded protein, and that the MITE+ is the drought-sensitive and MITE− is the drought-tolerant allele of ZmNAC111.

### Example 4: The MITE Causes the DNA and Histone Methylation at the ZmNac111 Locus

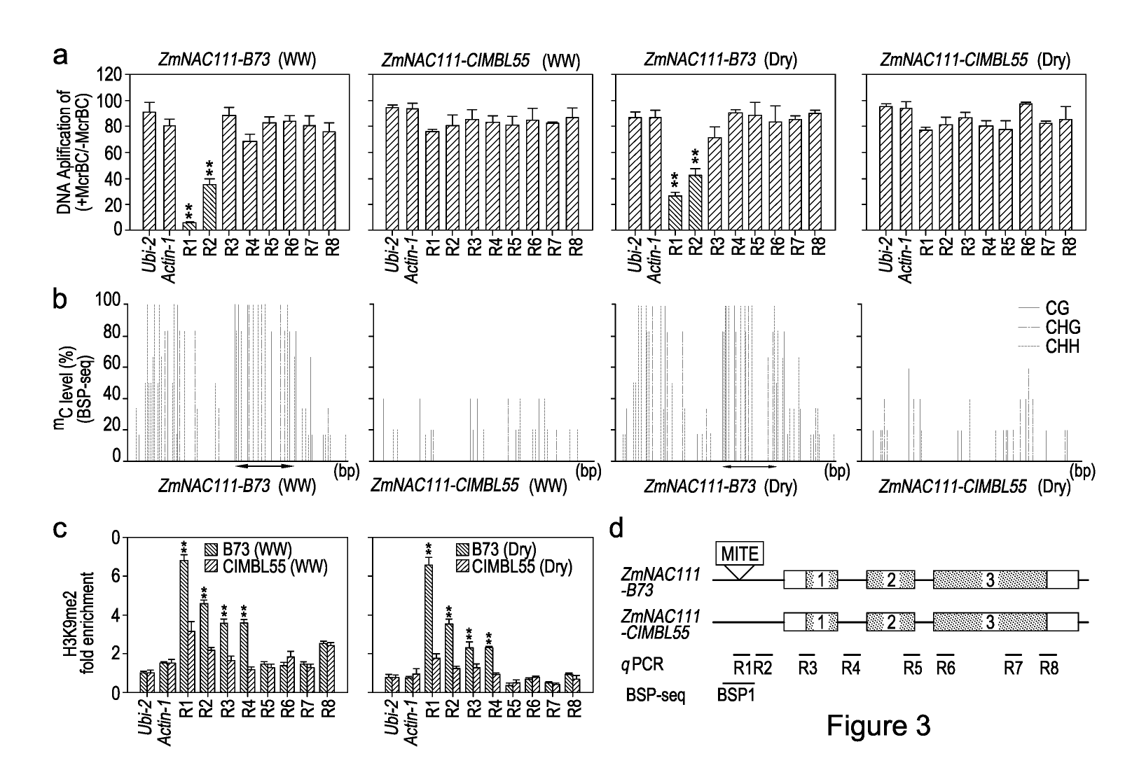

To understand how the 82-bp MITE reduces ZmNac111 expression, we then checked the DNA and histone methylation status of ZmNac111 in the MITE− and MITE+ genotypes. Eight regions (R1-R8) spanning the ZmNac111 gene and promoter in two inbred lines, B73 (drought-sensitive, MITE+) and CIMBL55 (drought-tolerant, MITE−), were representatively analyzed. Results revealed that only R1 and R2, nearest the MITE insertion, were hypermethylated in ZmNAC111-B73 but not in ZmNAC111-CIMBL55, regardless of the stress treatment (FIG. 3a). Bisulfite sequencing of these regions detected DNA hypermethylation, especially at the CHH content (FIG. 3b). Moreover, chromatin Immuno-precipitation (ChIP) using a specific H3K9me2 antibody and followed by qPCR analysis, demonstrated that H3K9me2 was significantly enriched in R1 to R4 in ZmNAC111-B73 in comparison with that in ZmNAC111-CIMBL55. The H3K9me2 levels of the other regions, however, remained comparable. Based on this data we suggest that the MITE insertion represses ZmNac111 expression through DNA and histone methylation of its nearby regions (FIG. 3c,d).

### Example 5: The MITE Represses the ZmNac111 Expression Through RdDM Pathway

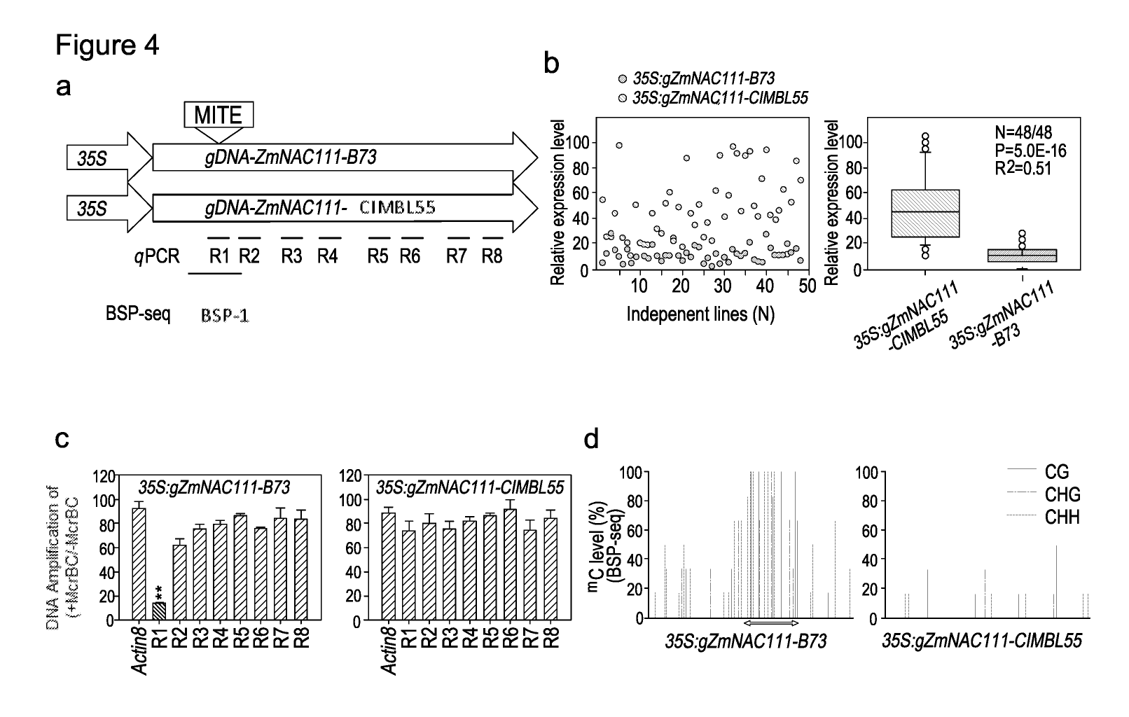

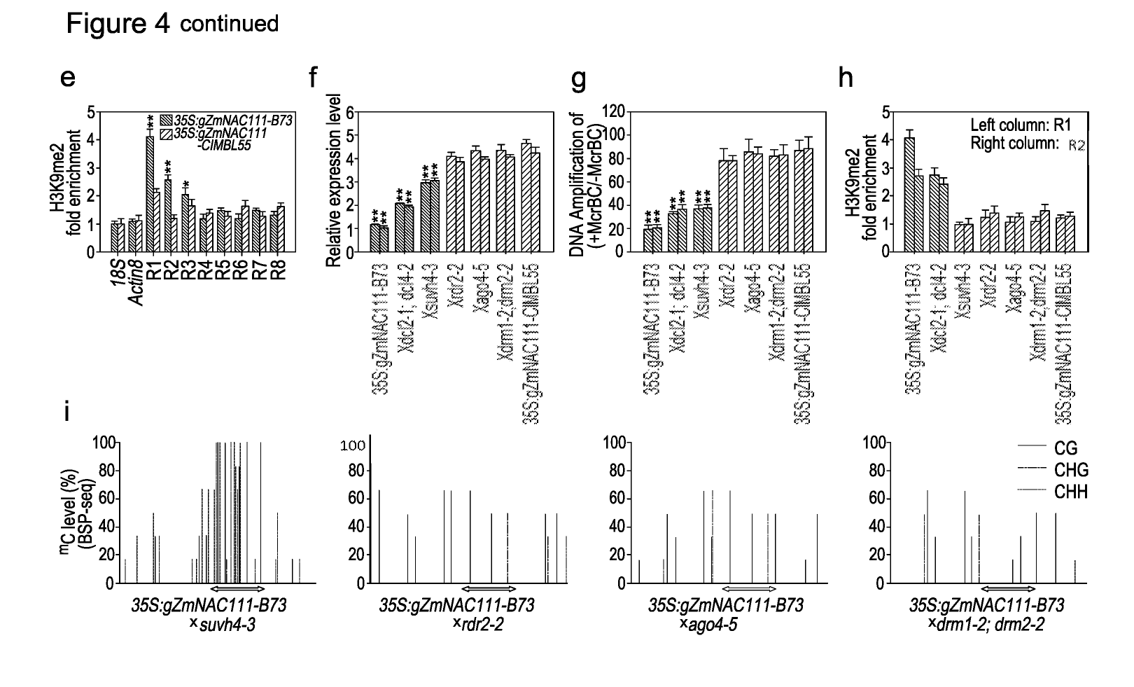

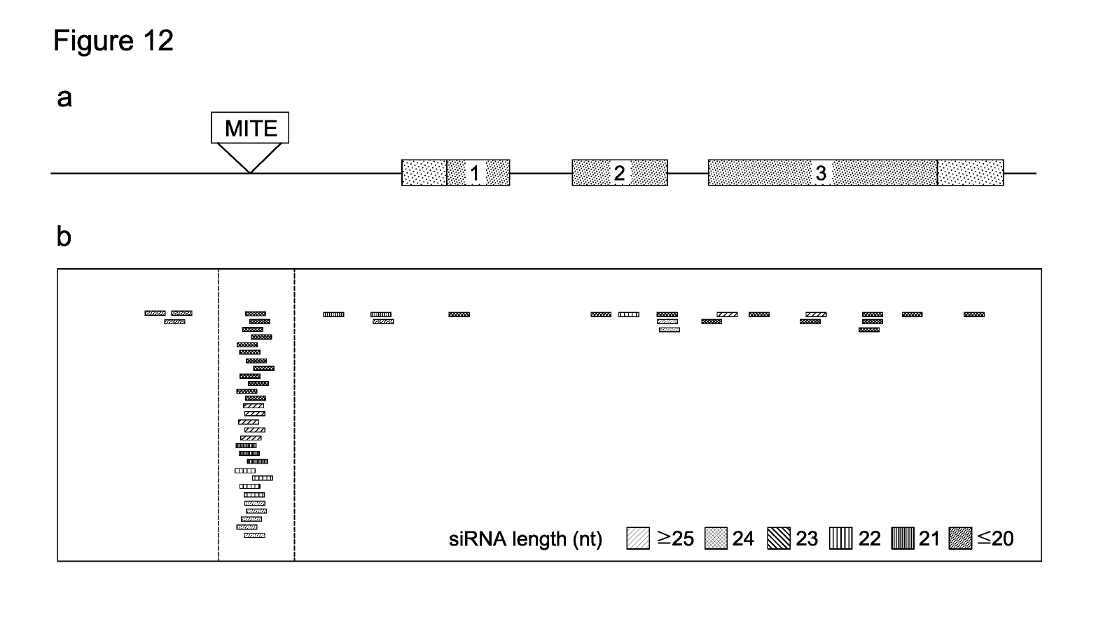

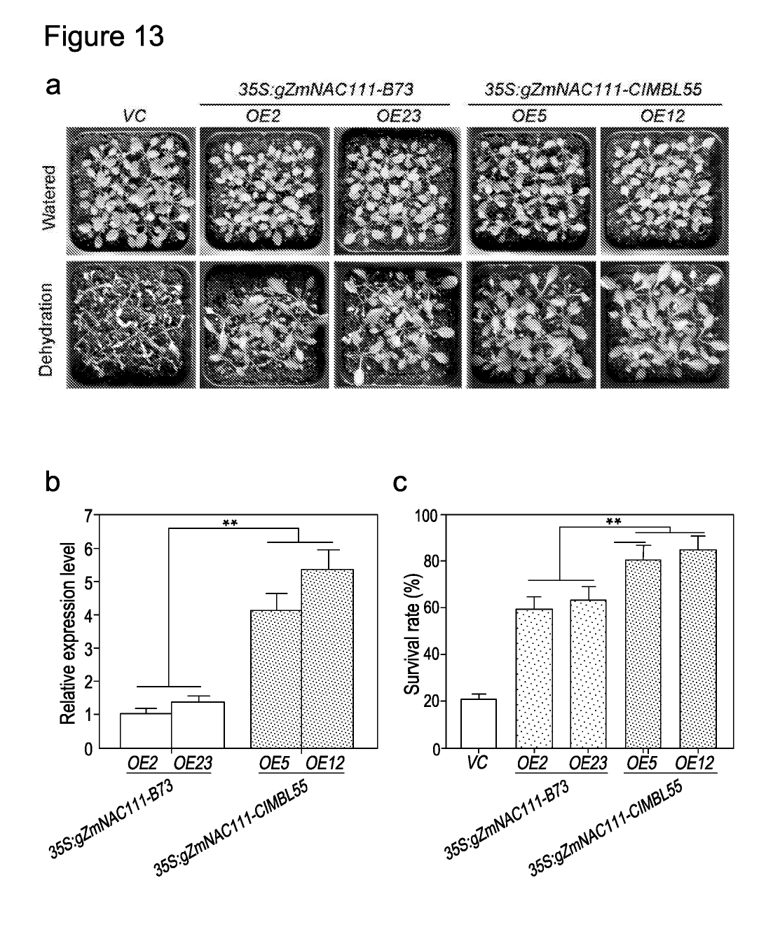

A considerable number of 21-nt to 24-nt small interference RNA (siRNA) species were identified that could be aligned to the MITE sequence in the maize small RNA database (http://sundarlab.ucdavis.edu/smrnas/) (FIG. 12). In an attempt to check the hypothesis that the 82-bp MITE insertion may mediate DNA and histone methylation through the RdDM pathway, the genomic fragments of gZmNAC111-B73 and gZmNAC111-CIMBL55 were transformed into Arabidopsis and expressed with the CaMV 35S promoter (FIG. 4a). The resultant 48 independent T2 transgenic lines for each construct were analyzed. The transgenic plants harbouring 35S:gZmNAC111-CIMBL55 generally exhibited significantly higher levels of ZmNac111 expression, compared with the 35S:gZmNAC111-B73 transgenics (FIG. 4b). DNA hypermethylation and H3K9me2 enrichment in the region nearby the MITE insertion were expectedly observed in the 35S:gZmNAC111-B73, but not in 35S:gZmNAC111-CIMBL55 transgenic lines (FIG. 4c-e). Plant drought tolerance was then compared between these two types of transgenics. The transgenic Arabidopsis harbouring 35S:gZmNAC111-CIMBL55 displayed greater drought tolerance than those transformed with 35S:gZmNAC111-B73 (FIG. 13).

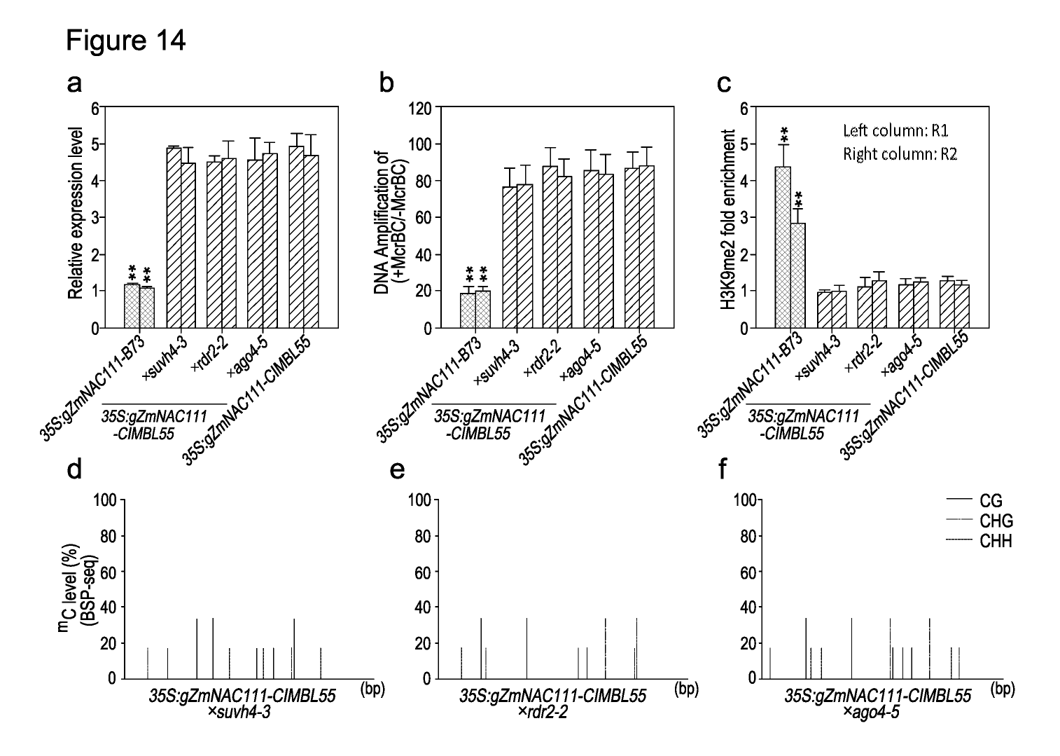

In order to verify that the MITE insertion mediated ZmNac111 repression through the RdDM pathway, five Arabidopsis mutants defective in key genes involved in the RdDM pathway were crossed with two independent homozygous 35S:gZmNAC111-B73-2 and -23 lines to transfer the 35S:gZmNAC111-B73 construct to the RdDM-defective backgrounds. In the drm1-2;drm2-2, ago4-5, and rdr2-2 mutants, ZmNAC111-B73 expression was greatly enhanced, while its DNA methylation and H3K9me2 were reduced to a comparable level relative to those of the ZmNAC111-CIMBL55 in the wild-type background. In the suvh4-3 mutant, the H3K9me2 level of ZmNAC111-B73 was significantly reduced, but the DNA hypermethylation remained relatively similar to that in the wild-type background, which was consistent with the fact that the SUVH4 functions as a histone methytransferase. In dcl2-1;dcl4-2, the gene expression, DNA methylation and H3K9me2 levels of ZmNAC111-B73 still differed from those observed for the ZmNAC111-CIMBL55 in wild-type background; most likely due to the intact DCL3 function in this mutant (FIG. 4f-i). No remarkable change in gene expression, DNA methylation and H3K9me2 level was observed when 35S:gZmNAC111-CIMBL55-5 and -12 transgenic Arabidopsis plants were crossed with the RdDM mutants (FIG. 14). Collectively, these data clearly indicated that MITE represses the ZmNac111 expression through the RdDM pathway when heterologously expressed in Arabidopsis.

### Example 6: Overexpression of ZmNac111 Confers Drought Tolerance

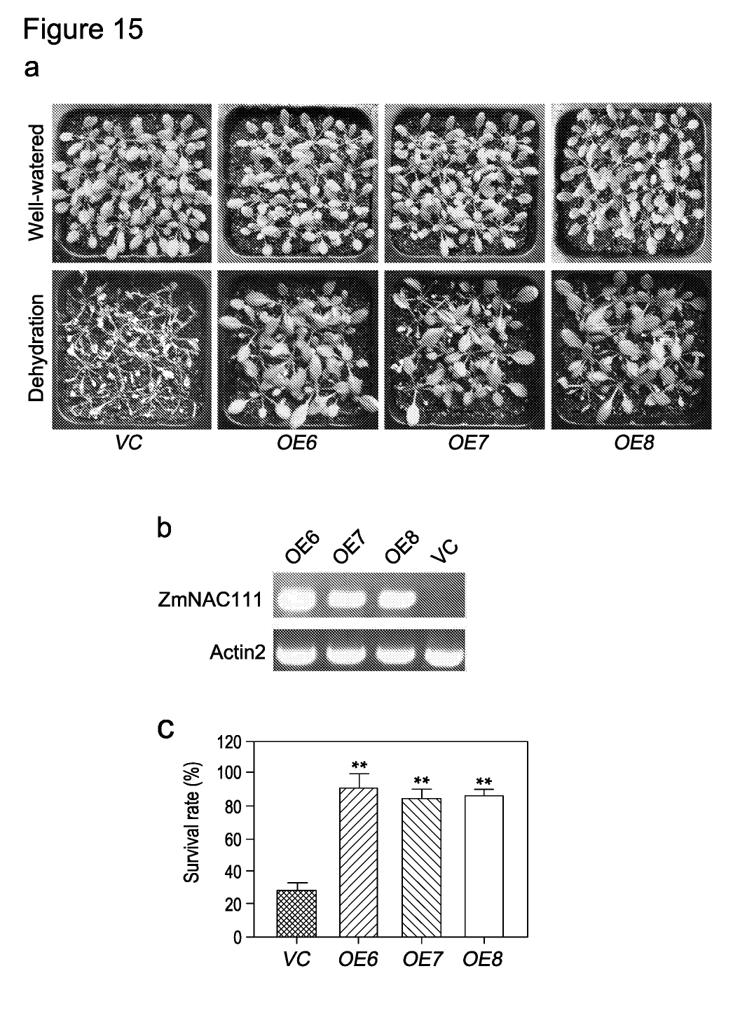

Given that ZmNac111 expression is positively correlated with maize drought tolerance, we generated both transgenic Arabidopsis and maize, overexpressing the coding sequence of ZmNac111 (from B73 genotype). For the transgenic Arabidopsis, the phenotypes of three independent 35S:ZmNAC111 lines were analyzed. In comparison with the empty-vector transformed plants (VC), the transgenic Arabidopsis displayed significantly enhanced drought tolerance, without remarkable morphological changes under normal growth conditions. When the survival of VC was around 20%, approximately 80% of the transgenic plants were alive in the parallel water-withholding experiments (FIG. 15a-c). The transgenic Arabidopsis plants were also hypersensitivity to exogenous ABA as shown by seed germination and stomatal closure assays, indicating an enhancement of ABA-signalling in the transgenic plants (FIG. 15e-g).

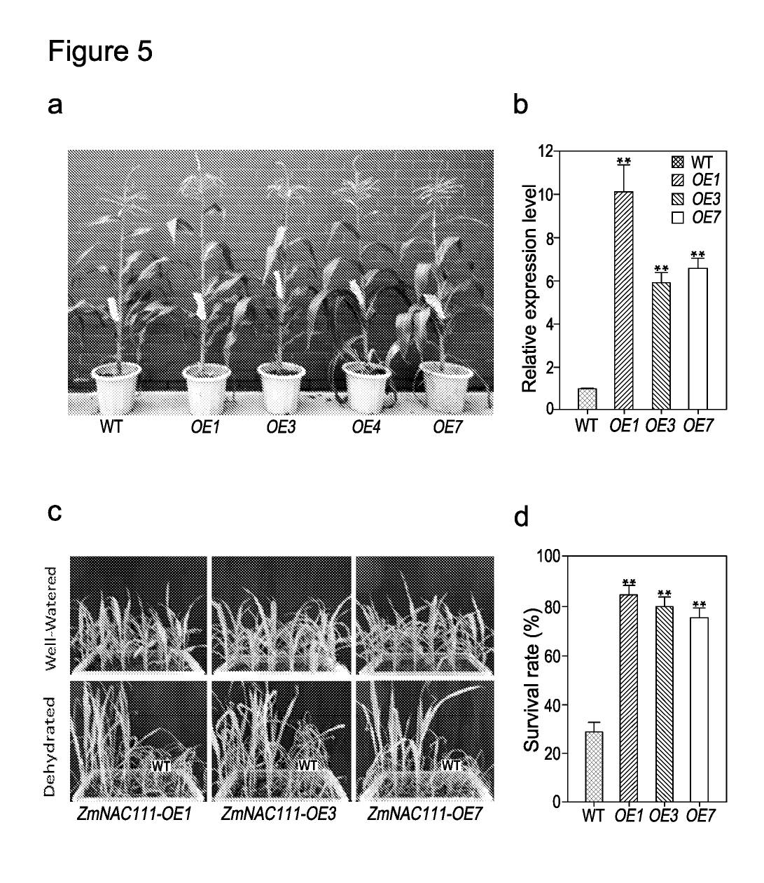

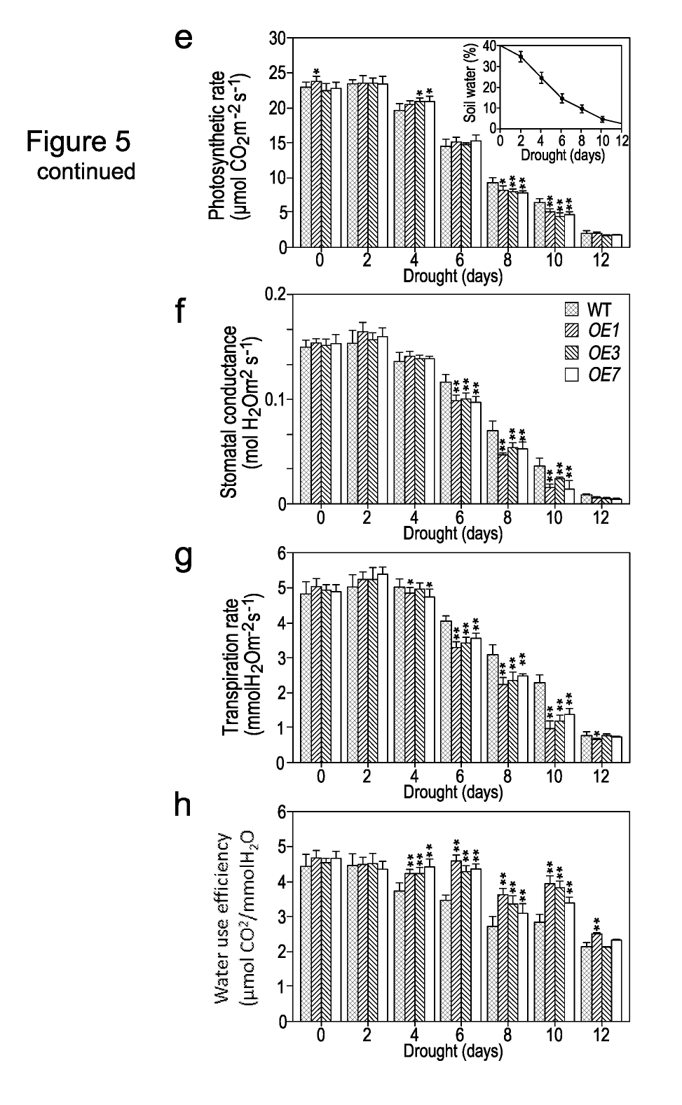

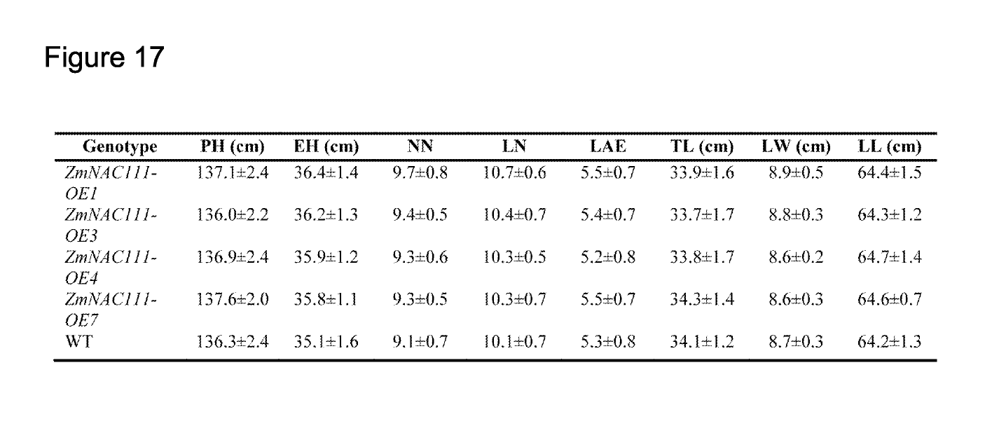

Similar improved drought tolerance was also observed in pot-grown transgenic maize transformed by ZmUbi:ZmNAC111. Under drought stress, approximately 80% of the T2 generation transgenic maize plants survived; whereas, the survival rate of the transgenic-negative sibling plants (WT) was only 30% (FIG. 5c,d). No evident abnormal changes were observed in the transgenic maize compared to WT under normal growth conditions, although we acknowledge that these growth conditions likely do not accurately represent field conditions (FIG. 5a,b and FIG. 17). Next, we compared the stomatal response and transpiration under progressive water stress between the transgenic maize and WT. The leaf photosynthesis rates (PS), stomatal conductance (SC), and transpiration rates (TR) were recorded every other day for 12 days in both WT and transgenic maize, when the soil water content (SWC) decreased from 40% to near 2%. During the first two days (SWC>30%), the three physiological parameters were comparable between the transgenics and WT, supporting the observation that they did not differ in growth under unstressed conditions (FIG. 5e-g). When SWC decreased to about 20%, TR of the transgenics was initially measured to decrease, resulting in a significantly greater WUE (calculated as PS in relation to TR) of the transgenic maize plants relative to WT (FIG. 5g,h). With the SWC dropping to ˜15%, the SC and TR became significantly smaller in the transgenics than those in WT, whereas, the PS remained comparable (FIG. 5e). Thus, WUE of the transgenic maize was maintained greater than that of the WT (FIG. 5g). Afterwards, the PS of the transgenics was more reduced compared with that of WT; however, due to a more remarkably reduced SC and TR, the WUE of the transgenics still remained significantly greater than that of WT, until SWC dropped to approximately 2% (FIG. 5h). These data revealed that the transgenic maize plants had a greater WUE under water deficit in comparison with WT, which conferred enhanced drought tolerance to the transgenic maize.

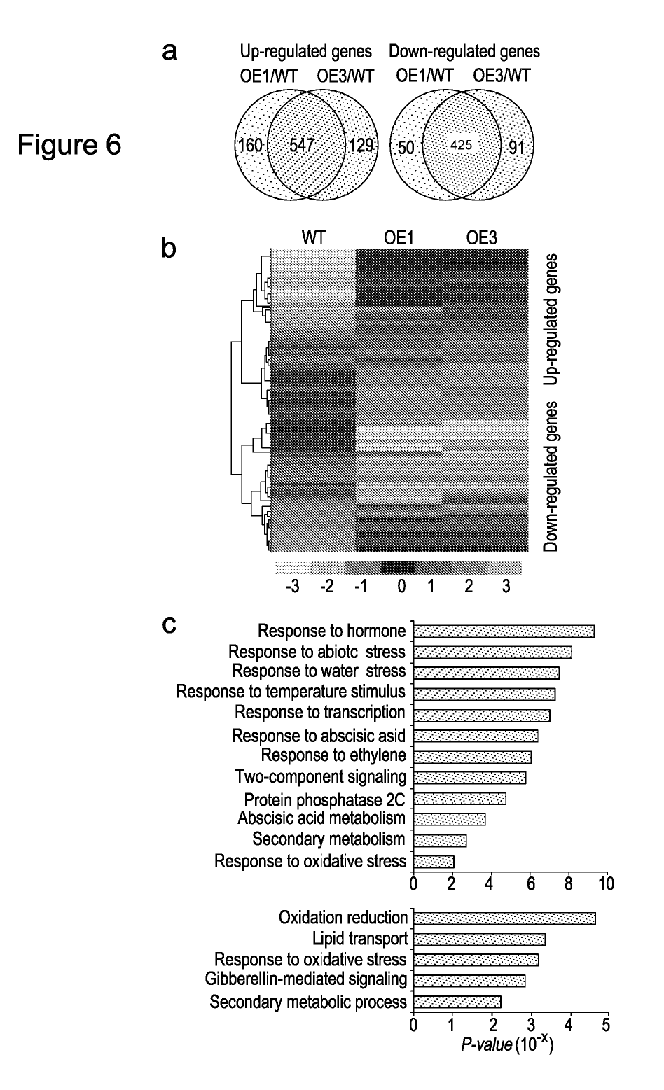

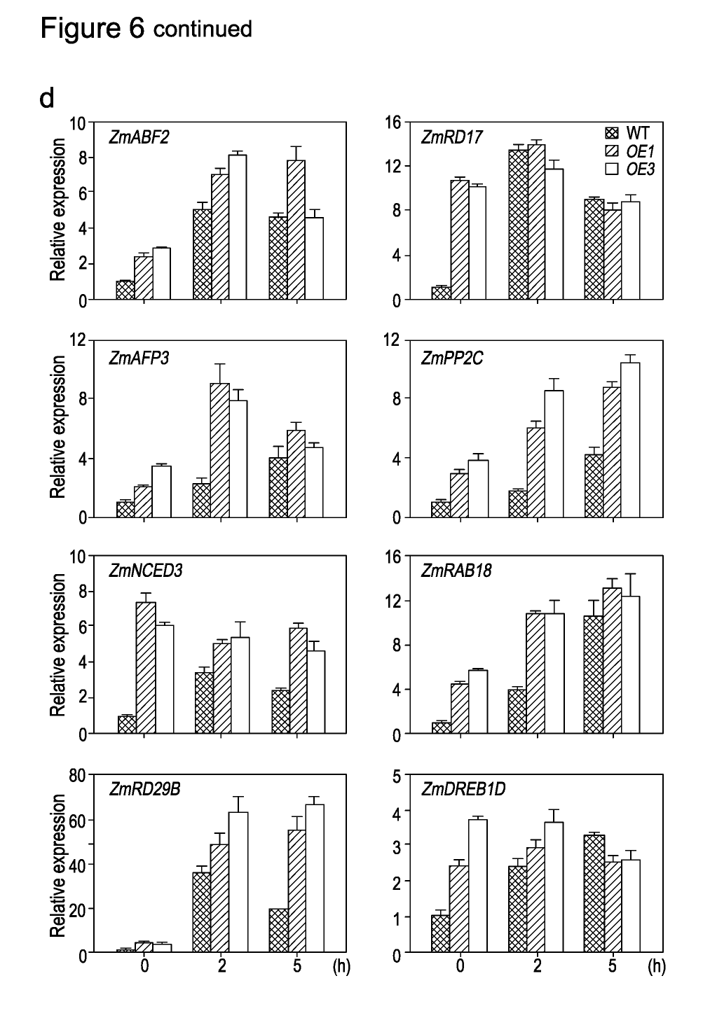

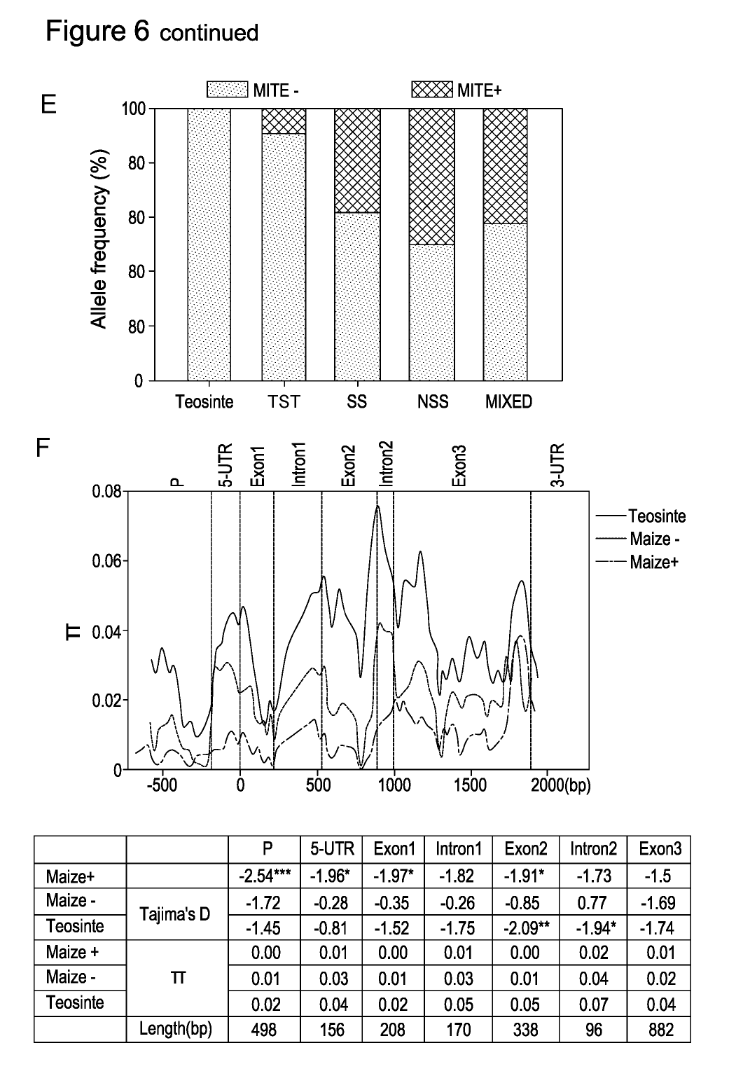

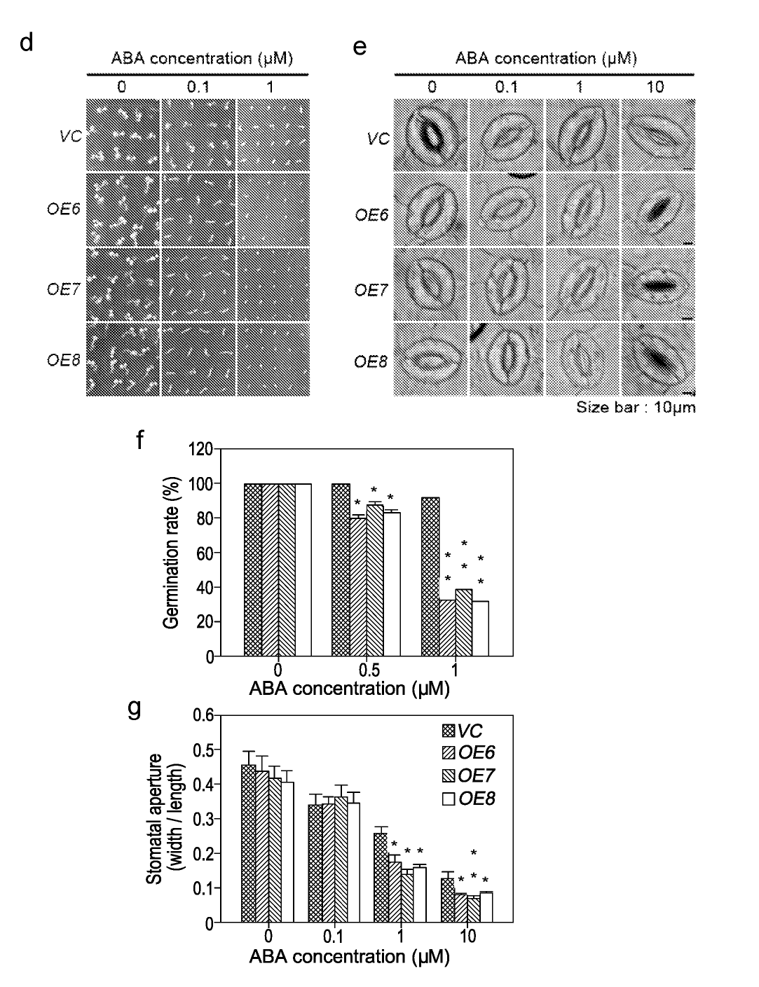

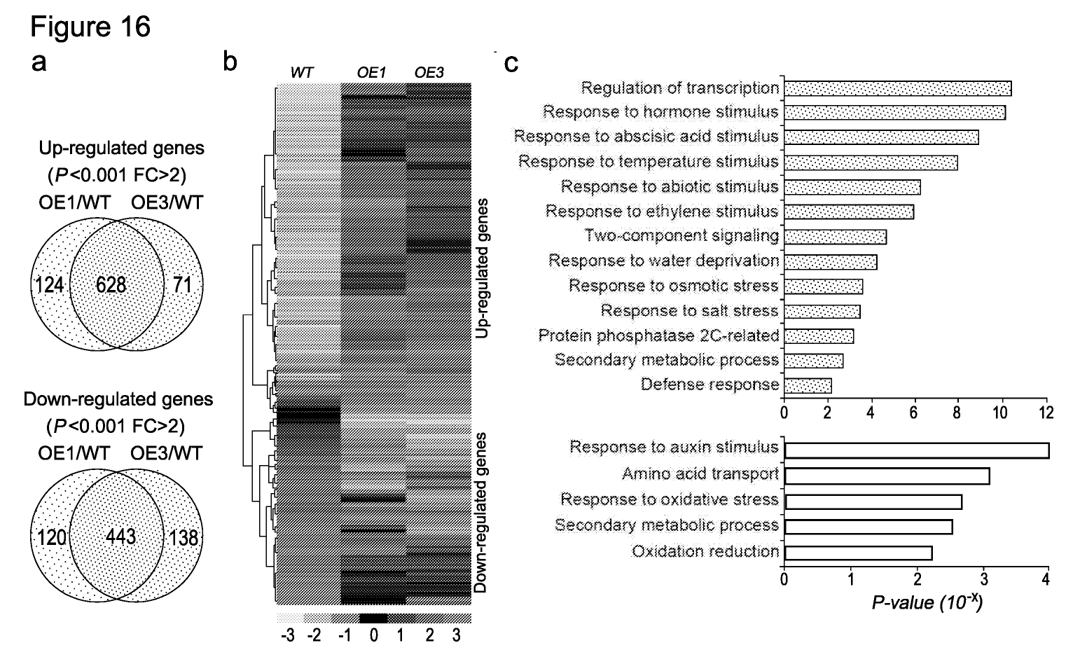

In the next line of our study, we compared the transcriptome of the transgenic maize and WT plants under favourable and drought conditions. A total of 628 and 443 genes were found to be up- and down-regulated by two-fold in the transgenics compared with WT, respectively, under well-watered conditions (FIG. 16). Under drought stress, 547 and 425 genes were 2-fold up- and down-regulated in the transgenic maize relative to WT, respectively (FIG. 6a,b). Biological pathways responsive to abscisic acid, ethylene and abiotic stimuli were greatly enriched amongst these identified up-regulated genes, whereas those responsive to oxidative changes and gibberellins were especially enriched amongst the down-regulated genes (FIG. 6c). Genes responsive to abiotic and water stresses were more significantly enriched in the up-regulated genes in the drought-stressed samples as compared with the untreated ones (FIG. 6c and FIG. 70c). These transcriptomic changes may contribute to the early reduction in TR, quick stomatal closure, and better protection of the photosynthesis machinery of transgenic maize under drought stress. Increased expression patterns of several well-known drought-inducible genes, such as the maize homologs of NCED343, AFP344, RAB1845, RD29B46, AHG147, RD1745, DREB1D48, were verified in the transgenic maize (FIG. 6d). Most of these genes contain copies of NAC recognition core sequence (CACG) [49,50] in their promoters, suggesting that they might be direct target genes of ZmNac111.

### Example 7: Evolutionary Aspects of the ZmNac111 Locus

Teosinte (Zea mays ssp.), a type of wild Mexican grass, is recognized as the direct progenitor of maize, based upon the fact that the natural cross of teosinte and maize are fertile and the availability of a wealth of genetic domestication information8,9. After domestication from teosinte, maize cultivation spread from TST to temperate regions and maize became a major crop plant providing nutritional calories for consumption by human beings. We were interested to know the presence and distribution of the 82-bp MITE insertion in the teosinte ZmNac111 locus. When 96 teosinte accessions were genotyped, none of them were found to carry the MITE insertion at ZmNAC111, indicating that this insertion might have occurred after the domestication of maize. Moreover, among 116 TST inbred lines, 10.34% of them were MITE+. Whereas, among 146 temperate lines (including stiff-stalk (SS), non-stiff-stalk (NSS), and Mixed origins51), 41.78% were MITE+(FIG. 6e). Nucleotide diversity at the ZmNac111 locus was found to decrease from teosinte to TST and then to temperate maize (FIG. 6f). This finding suggested that with the spread of maize cultivation from TST to temperate regions, the MITE+ genotype was accumulated especially in temperate maize germplasm. The MITE insertion in ZmNac111 locus may compromise drought tolerance of temperate maize varieties, which is in agreement with the observation that temperate subpopulation was averagely more susceptible to drought than the TST subpopulation in the whole natural variation population. Thus, we propose that the selection of the MITE− genotype may help to improve drought tolerance in temperate maize inbred lines.

### Example 8: RNA-Seq Analysis of Transformed ZmNAC111 Arabidopsis

The results are shown in FIG. 26 and FIG. 27. The results demonstrate that in the transgenic Arabidopsis that grew under normal conditions and under drought treatment, the expressions of genes related to the responses to water stress, abscisic acid (ABA) stress and genes of transcriptional regulation associated with biological pathways were modulated.

The above results show that protein ZmNAC111 and its encoding genes possess the function of regulating the drought tolerance of a plant. The overexpression of the encoding gene of the protein ZmNAC111 in plants can improve the drought tolerance of plants.

### DISCUSSION

In this research, we reported that the natural variation of ZmNac111 gene is associated with maize drought tolerance on the whole genome-scale. By using a combined approach of GWAS and transgenic studies, we demonstrated that the causative variation at the ZmNac111 locus is likely an 82-bp MITE insertion in the gene promoter, which represses the gene expression through DNA and histone hypermethylation via the RdDM pathway. Our findings highlight the likely regulatory function of a TE in maize stress response and provide important insights into the genetic basis of the natural variation in maize drought tolerance; as well as new genetic strategies for improving this trait.

We identified the 82-bp MITE insertion in a location that was 572-bp upstream of the ZmNac111 coding region which was correlated with lower ZmNac111 expression and drought susceptibility (FIG. 2b). Bisulfite-seq and ChIP-qPCR analyses revealed that DNA and histones are hypermethylated in the ZmNac111 locus in the maize inbred lines carrying the MITE (FIG. 3). The repression of ZmNac111 expression could be reproduced when the genomic fragment containing the MITE and ZmNac111 was transferred into Arabidopsis, indicating that the underlying molecular mechanism is likely conserved across plant species. Importantly, using the Arabidopsis RdDM-defective mutants, we demonstrated that the MITE-mediated ZmNac111 repression, at least when heterologously expressed, is dependent on RdDM (FIG. 4); which is a well-characterized RNA interference-related transcriptional gene silencing mechanism in plants [32]. This modification induces and reinforces transcriptional silencing of TEs, as well as the genes that harbor or are adjacent to the TEs26. Recently, whole-genome DNA methylation and RdDM surveys in maize suggest that 24-nt siRNAs are much more highly associated with transposons, which tend to be close to genes rather than the heterochromatin regions [52]. Although the majority of the maize genome exists in a heterochromatic status which is marked by H3K9me2 and H3K27me2, RdDM was only observed to be near gene-coding regions [53]. As a result, it gave rise to the formation of CHH islands predominately near genes, rather than in the repetitive intergenic DNA regions [52,53]. Our findings provide a potential molecular mechanism for how the 82-bp MITE interplays with its adjacent gene so as to contribute to drought tolerance variance in the natural maize population.

TFs play important roles in the regulation of gene expression in response to abiotic stresses, and their molecular engineering is proposed as a potential strategy for the genetic improvement of stress tolerance in crops [54,55]. NAC proteins constitute a plant-specific superfamily whose members participate in various regulatory and developmental processes, including stress response and tolerance [42]. The typical NAC proteins share a conserved N-terminal DNA-domain, but vary greatly in other regions; resulting in distinct functions of different proteins. In maize, at least 116 predicted NAC members have been identified [35]. Although ZmNac111 was classified into an identical phylogenetic clade with OsNAC10 among the annotated NAC proteins examined (FIG. 8), ZmNac111 and OsNAC10 only share a 48% sequence identity on the full protein level, indicating both functional similarity and diversity between them. Previous reverse genetic studies reported that increasing OsNAC10 expression in roots improved the yield of transgenic rice under drought [36]. Expression analysis of ZmNac111 in leaf samples of 133 natural maize varieties indicated that ZmNac111 expression was positively correlated with drought-tolerance under moderate and severe stresses (FIG. 2). These data suggested a positive regulatory role of ZmNac111 in maize seedlings exposed to water stress. Moreover, transgenic studies in both Arabidopsis and maize demonstrated that overexpression of the ZmNac111 gene could improve drought tolerance of transgenic plants by modulating the stomatal closure and drought-responsive gene expression. These observations strengthen our findings that the elevated expression of ZmNac111 gene contributes to maize drought tolerance and that ZmNac111 acts as a positive regulator of drought response in maize (FIG. 5 and FIG. 15). Comparative transcriptome analysis of the transgenic maize and WT determined that a number of genes involved in ABA-responsiveness, such as ZmNCED3, ZmRAB18, ZmRD29B, ZmRD17 and ZmPP2C, were upregulated; indicating that ZmNac111 might function in an ABA-dependent stress-responsive pathway (FIG. 6d). In agreement with this result, Arabidopsis transgenic plants were more sensitive to exogenous ABA treatment in regards to germination and stomatal closure (FIG. 15). In transgenic maize, the leaf SC and TR were more responsive to water deficit during the decrease of SWC, from 15% to ˜2%, in the drought treatment (FIG. 5e-h). On the basis of these results we suggest that ABA-dependent regulation was enhanced in the ZmNac111 transgenic plants.

It is considered that enhancing effective use of water (EUVV), which implies maximal soil moisture capture, reduced non-stomatal water loss and management for minimal soil evaporation, is important for drought tolerance improvement under field conditions; however, improving WUE by reducing SC and TR may diminish plant yield in fields [56]. In this research, we did not observe obvious morphological changes in transgenic plant overexpressing ZmNac111 in comparison with the control plants, when they grew in pots under favorable greenhouse conditions (FIG. 5a). It was supported by the comparable measurements of the leaf PS between the transgenic and WT plants (FIG. 5e). Only upon drought stress, a greater WUE was revealed in transgenic maize in comparison with WT (FIG. 5h). A similar phenomenon was also observed in transgenic rice overexpressing SNAC150. The early reduction of TR was observed in transgenic maize, when the SC remained comparable between the transgenic maize and WT (SWC>20%); which was probably due to a better or quicker osmo-adjustment (FIG. 5g). In spite of the more highly reduced SC and TR in the transgenic maize as compared with WT, the leaf PS was hardly affected when SWC was ˜15% (FIG. 5e,f). These results indicated a better protection of photosynthesis machinery or maintenance of cellular oxidative status under drought stress in the transgenic maize. Thus, the improved drought tolerance and greater WUE of transgenic maize were likely attributed to enhanced ABA signalling, quicker osmo-adjustment, better cellular protection in response to drought stress, rather than the consequence of plant growth retention. These findings also suggest that efficient water usage of plants can be improved both physiologically and genetically. Nevertheless, further intensive evaluations on important agronomic traits of transgenic plants under field conditions are needed with regard to the concerns of gene application to maize production.

In addition to SNPs, TE presence/absence variations are common and widely distributed in the maize genome, which is considered a driving force for crop evolution and domestication [57]. The MITE insertion was only present in maize germplasm but not in the teosinte accessions we examined (FIG. 6e). This finding suggests that the MITE may have inserted into ZmNac111 locus after maize domestication from its wild ancestor. The domestication of crops from their wild ancestors may cause the loss of genes or alleles which are responsible for tolerance to various environmental stresses. Recently, it has been reported that the deletion of the ZmWAK gene during maize domestication increases susceptibility of domesticated cultivars to head smut which is a major disease in maize production [12]. Decrease in plant stress tolerance might be exaggerated if the stress pressure is not present during the selection in breeding program, in which high yield, but not stress tolerance, is the primary goal. No evident adverse effect was observed on plant normal growth and development of the ZmNac111 transgenic Arabidopsis and maize under the favourable conditions. However, further investigation is required to test if enhancement of the ZmNac111 expression results in additional undesired phenotypes in field conditions. In addition, the ZmNac111 locus was not found to be associated with 17 important agronomic traits by analyzing 513 maize inbred lines [58]. Therefore, ZmNac111 is may be a potential candidate in gene engineering, and its MITE− allele could be a selection target for the genetic improvement of drought tolerance in maize. It should be noticed that in the current study, maize drought tolerance was evaluated at seedling stage in pot-cultivated plants, which limited the characterization of above-ground tissues and vegetative growth. Whether ZmNac111 and its MITE− allele can significantly contribute to maize yield under drought in fields demands further field-based investigation. Additionally, the MITE insertion seems to be especially common in temperate maize germplasm and whether this allele confers any advantage in breeding programs aimed for temperate regions may be an interesting theme for future research.

Methods

ZmNAC111-Gene Association Mapping.

GWAS of maize drought-tolerant genes was performed by analyzing a maize natural variation panel consisting of 368 inbred lines collected from TST and temperate regions19. Plant drought tolerance of different inbred lines was phenotypied as previously described24. Briefly, the natural variation panel of maize consisting of 368 maize inbred lines was planted in a cultivation pool (6×1.4×0.22 m, length×width×depth) in which 5-ton of loam were mixed with 0.25-ton of chicken manure. To phenotype the drought tolerance of each genotype, watering was withheld when the seedlings developed three true leaves. Re-watering was applied to recover the surviving plants when clear wilting difference was observed. After rehydration for 6 days, the survival rate of each genotype was scored. The phenotypic data were obtained from 6 replicated experiments. The 56,110 genomic and 525,105 transcriptomic SNPs, with minor allele frequency (MAF)≥0.05, were used for GWAS. The standard mixed linear model was applied (TASSEL 3.1.0)59, in which the population structure (Q) and kinship (K) were estimated as previously described24. Briefly, principle components of the association panel were calculated by EIGENSTRAT60 using the high-quality 52,5105 SNP data with MAF 0.0561. The first two dimensions were used in the principle component analysis (PCA) to estimate the population structure, which could explain the 11.01% of the phenotypic variation. These results were comparable to those that were calculated by STRUCTURE. The analysis was completed by the Im function in an R program. Single-marker association analysis was initially performed to filter out markers that had no relationship with the trait (p 0.995). Subsequently, 1,822 SNP markers on each chromosome were chosen to estimate the kinship coefficient (K) by SPAGeDi. Markers that were in approximate linkage equilibrium with each other were determined from PLINK62 based on SNP pruning (window size 50, step size 50, the LD R2 threshold is 0.2) and the number of the subset markers was 85,806. The suggestive P-value threshold to control the genome-wide type 1 error rate was 1.17×10-5, which was considered as the significance cutoff for the association. ZmNAC111-based association mapping was performed within 146 temperate and 116 TST maize inbred lines, which were representative of the whole population. The ZmNac111 promoter (˜0.7 kb), coding regions (include introns), and 5′ and 3′-UTR sequences were amplified and sequenced. These sequences were assembled using ContigExpress in Vector NTI Advance 10 (Invitrogen) and aligned using MEGA version 5 (http://megasoftware.net/). Polymorphisms (SNPs and InDels) were identified and their association to drought tolerance was calculated again by TASSEL 3.1.0, under the standard MLM, with MAF 0.05.

ZmNAC111 Gene Expression Analysis in Different Inbred Lines.

ZmNAC111 expression was analyzed in 133 maize inbred lines. Drought treatment was applied to the soil-grown plants at the 3-leaf seedling stage by withholding water. Leaf samples were collected when the relative leaf water content (RLWC) decreased from approximately 98% to 70%, and to 58%. Total RNA of 399 samples was isolated using TRIZOL reagent (Biotopped) from a minimum of 3 seedlings. RNA was treated with RNase-free DNasel (Takara), and single-stranded cDNA was synthesized using Recombinant M-MLV reverse transcriptase (Promega). The quantification method (2^{-ΔCt})⁶³ was used and the variation in expression was estimated using three biological replicates. The maize Ubi-2 (UniProtKB/TrEMBL; ACC: Q42415) gene was used as an internal control to normalize the data. PCR conditions consisted of an initial denaturation step at 95° C. for 10 min, followed by 40 cycles at 95° C. for 15 sec, 60° C. f0 sec.

Allelic Effect of ZmNac111 in Maize Segregating Populations.

Three F2:3 segregating populations (CIMBL91×BY4944, CIMBL55×GEMS54 and CIMBL55×CIMBL9) were constructed. The genotype at the ZmNac111 locus was analyzed in approximately 200 individual F2 plants in each population. Polymorphisms in the PCR products were visualized on 2% agarose gels. Homozygous F2 individuals at the ZmNac111 locus of the tolerant allele (MITE−) and the sensitive allele (MITE+) were self-pollinated to obtain F3 progenies. F3 progenies that were homozygous at the InDel-572 locus (MITE−/− or MITE+/+) were mixed, respectively. Two types of F3 plants were grown in enriched soil (soil to vermiculite in a ratio of 1:1) in plastic boxes (0.70×0.50×0.18 m, length×width×depth) and their drought tolerance was evaluated. Each box contained 90 seedlings for each type of F3 plants. Three independent replications were performed in a greenhouse using 16-h-light/8-h-dark, 28/22° C. and a room humidity of 60% to obtain the statistical data. Drought was applied to the 10-day-old plants by withholding water. When SWC decreased from 40% to near 0%, and wilting and death of the seedlings were visible, plants were re-watered in order to identify surviving plants. The survival rate of each genotype was recorded. Three replications were carried out for statistical analyses.

The Acquisition of the Protein ZmNAC111 and its Encoding Gene

Corn inbred line B37 seeds were germinated under 28° C. for three days, and then the budding seeds were transferred to soil or solution with nutrients for 3-week cultivation. The whole plant was quick frozen and grinded, and then the total RNA was extracted and subjected to an inverse transcription so as to obtain cDNA. Then PCT amplification was performed with the cDNA as the template and 5′-ATGCCGAGAAGCGGCGGCG-3′ (SEQ ID NO. 29) and 5′-CTACTGCATCCCGATGTGGC-3′ (SEQ ID NO. 30) as the primers. The amplified products were subjected to an agarose gel electrophoresis and 1.4 kb PCR amplification products were obtained. After sequencing, the PCR products have the nucleic acid shown by locus from 157-1584 in SEQ ID NO. 28, the gene is denoted as ZmNAC111; the protein encoded by said gene is called ZmNAC111; and the amino acid sequence of the protein is SEQ ID NO. 27. In SEQ ID NO. 28, locus from 1 to 156 is the 5′ non-coding region, locus 157-1584 is the coding sequence and locus 1585-1824 is the 3′ non-coding region.

Transcriptional Activation Activity Analysis for Different Monomeric Coded Protein ZmNAC111

Drought tolerant corn inbred lines CIMBL55, CIMBL91, CIMBL19, CIMBL22 and CIMBL23 and sensitive corn inbred lines Mo17, D863F, BY4944, SHEN5003, which carry different genotypes, as well as CDNA of corn inbred line B73 serve as the templates.5′-ATGCCGAGAAGCGGCGGCG-3′ (SEQ ID NO. 29) and 5′-CTACTGCATCCCGATGTGGC-3′ (SEQ ID NO. 30) were used as primers for PCR amplification; the target gene clones were incorporated into the yeast expression carrier pGBKT7, and respectively transformed into the yeast strain AH109 (containing the reporter genes HIS3 and ADE2). The transformation of empty vector pGBKT7 served as the reference and recombinant yeast strains ZmNAC111-CIMBL55, ZmNAC111-CIMBL91, ZmNAC111-CIMBL19, ZmNAC111-CIMBL22, ZmNAC111-CIMBL123, ZmNAC111-Mo17, ZmNAC111-D863F, ZmNAC111-BY4944, ZmNAC111-SHEN5003, ZmNAC111-B73 and pGBKT7-Control were obtained respectively. AH109 recombinant yeast strain was coated on the plate of nutrition-deficient medium. Then, the transcription activation activity of ZmNAC111 encoded by different monotypes was compared with the growth of the bacterial plaque.

As shown in FIG. 11, all 11 recombinant yeast stains can grow on the plate of SD-Trp (mono-deficiency) medium. Yeast strains containing pGBKT7-Control plasmid cannot grow normally while all recombinant yeast strains can grow normally on SD/-T-H (bi-deficiency) and SD/-T-H-A (triple-deficiency) nutrition-deficient medium, which indicates that protein ZmNAC111 encoded by different monotypes possess similar transcription activation activity.

McrBC-Based DNA Methylation Assay.

Genomic DNA was isolated from fresh young leaves collected from B73 and CIMBL55 maize lines before (RLWC=98%) or after drought treatment (RLWC=70%). DNA (1 μg) was digested for 16 hrs at 37° C. with 10 units of McrBC enzyme, a DNA methylation sensitive enzyme (Takara), in parallel with a mock reaction. 50 ng of digested DNA was used for qPCR reactions. DNA hypermethylation was demonstrated by the lower amount of amplification products in the qPCR analysis. All results were obtained by digesting at least two biological replicates and two independent McrBC digests. qPCR was performed using the following conditions: step 1: 95° C., 10 min; step 2: 95° C., 15 sec, 60° C., 30 sec (40 cycles). McrBC digestion at the ZmNac111 gene was normalized to the reference gene maize Ubi-2 and Actin1 and then to the undigested control. Arabidopsis plants were grown on MS agar plates for twenty-one days prior to collection. Actin8 was used as the reference gene in Arabidopsis64. Digestion levels have been inverted to represent methylation levels.

Bisulfite Analysis.

Bisulfite treatment was performed on 200 ng of genomic DNA by using the EZ DNA Methylation-Gold Kit (Zymo Research, Orange, Calif.). After bisulfite conversion, the treated DNA was amplified by PCR. Amplified fragments were cloned into the pGEM-T vector (Promega) for sequencing. At least eight clones of each genotype were sequenced.

ChIP Assay.

Fresh young leaves were collected from B73 and CIMBL55 maize lines grown under normal and drought conditions as described above. Whole plants of Arabidopsis were collected from 35S:ZmNAC111-B73 and 35S:ZmNAC111-CIMBL55 lines grown on the MS agar plates for twenty-one days. ChIP assays were performed as previously described65. 7 μl antibodies of anti-H3 (Abcam; ab1791) or anti-H3K9me2 (Abcam; ab1220) were used for the ChIP assays. The amount of immunoprecipitated ZmNac111 chromatin was determined by qPCR on different regions of the ZmNac111 locus. Maize Ubi-2 and Actin8 were used as internal controls for maize and Arabidopsis, respectively. The relative abundance was normalized to the amount of DNA immunoprecipitated by a Histone 3 specific antibody.

Generation Transgenics in the RdDM Mutant Backgrounds.

Two 35S:gZmNAC111-B73-2, -23 and 35S:gZmNAC111-CIMBL55-5, -12 homozygous T2 lines were crossed with five mutants: dcl2-1;dc13-1;dcl4-2 (CS16391), drm1-2;drm2-2 (CS16383), ago4-5 (CS9927), rdr2-2 (SALK_059661), and suvh4-3 (Salk_105816) (http://www.arabidopsis.org). Through PCR-based genotyping analysis, at least three independent F2 homozygous plants for each cross were obtained and harvest. Since the DCL3 locus was heterozygous, only 35S:gZmNAC111-B73-2 and -23 in the dcl2-1;dcl4-2 mutant background were obtained. The ChIP assay was performed using F3 RdDM-mutant homozygous plants obtained from crossing the two independent transgenic Arabidopsis with the RdDM mutants. The F3 plants germinated on Kanamycin-selective medium were used for further DNA methylation and ChIP analyses. Arabidopsis T-DNA insertion lines were obtained from the Arabidopsis Biological Resource Center.

Drought Tolerance of the Transgenic Arabidopsis

The ZmNac111 genomic region in the B73 and CIMBL55 inbred lines, and the coding region in B73, were amplified and inserted into the pGreen vector66 under the CaMV 35S promoter using the NotI and XhoI restriction sites. The constructed plasmid was transformed into the GV3101 Agrobacterium tumefaciens strain containing the pSoup helper plasmid. Arabidopsis thaliana ecotype Col-0 was transformed by Agrobacterium-mediated transformation and independent T2 transgenic lines were obtained using kanamycin-based selection. ZmNac111 gene expression in transgenics was determined by qRT-PCR, in which Actin8 was used as an internal control for normalization. For the drought tolerance assays, seven-day-old plants were transferred into pots containing 250 g of soil. Thirty two-day-old plants growing under favorable water conditions were exposed to drought stress. Water was withheld from the plants for 14 days. Watering was then resumed to allow the plants to recover. Six days later, the number of surviving plants was recorded. At least 64 plants of each line were compared with empty-vector transformed (VC) plants in each test, and statistical data were based on data obtained from three independent experiments.

The Acquisition of ZmNAC111 Arabidopsis

Recombinant tumefaciens X was used to transform the wild type Columbia ecotype Arabidopsis by using flower bud immersion method to obtain T1 generation seeds; T1 generation seeds were screened by using MS medium containing 30 mg/L kanamycin and then the seedlings showing the resistance to kanamycin were cultivated and harvested to obtain T2 generation seeds; T2 generation seeds were screened by using MS medium containing 30 mg/L kanamycin, kanamycin-resistant seedlings showing the kanamycin-resistance segregation ratio of 3:1 were selected to be T2 generation transformed ZmNAC111 Arabidopsis.

The RNA of T2 generation transformed ZmNAC111 Arabidopsis was extracted and was reversely transcribed to obtain cDNA as the template. The cDNA of gene ZmNAC111 was then subjected to PCR amplification with the use of specific primers F1 and R1, wherein the gene of Actin2 of Arabidopsis was an internal reference and the primers were FC and RC.

The sequences of the above-mentioned primers are as follow:

F1:
(SEQ ID NO. 29)
5′-ATGCCGAGAAGCGGCGGCG-3′;
R1:
(SEQ ID NO. 30)
5′-CTACTGCATCCCGATGTGGC-3′;
FC1:
(SEQ ID NO. 31)
5′-GGTAACATTGTGCTCAGTGGTGG-3′;
RC1:
(SEQ ID NO. 32)
5′-GCATCAATTCGATCACTCAGAG-3′.

1428 bp positive T2 generation transformed ZmNAC111 Arabidopsis was thus obtained.

Positive T2 generation transformed ZmNAC111 Arabidopsis was harvested to obtain T3 transformed ZmNAC111 Arabidopsis seeds which were then screened by using MS medium containing 30 mg/L kanamycin to obtain 3 homozygosis T3 transformed ZmNAC111 Arabidopsis strains, which were respectively named as TL1, TL2 and TL3 and these strains presented no kanamycin resistance segregation.

A similar method was applied to use the recombinant tumefaciens CK to transform the wild type Columbia ecotype Arabidopsis by flower bud immersion method. Based on the above screening method, homozygosis T3 transformed empty vector Arabidopsis that presents no kanamycin resistance segregation was obtained and named as CK or VC (empty vector).

T1 generation represents seeds obtained from transforming the general generation and plants grown from them; T2 generation represents seeds obtained from transforming T1 and plants grown from them; T3 generation represents seeds obtained from transforming T2 and plants grown from them.

Specific procedures of the above-mentioned flower bud immersion were as follow: Recombinant tumefaciens X or CK was inoculated in LB liquid medium containing 50 mg/L kanamycin and 5 mg/L tetracycline, and was incubated at 28° C. under shaking until OD600 reached 0.8. The mixture was then centrifuged at 25° C., 5000 rpm for 2 minutes to remove the supernatant. The thallus was re-suspended with a re-suspending solution (the solvent was water and the concentration of the solute of sucrose and silwet77 was 50 g/L and 0.02% (volume percentage) respectively) to obtain a dipping solution. The flower bud and the growing points of the plant were dotted with the dipping solution by a pipette and were covered with thin films. After moisturizing the flower bud and the growing points of the plant for 2 days, they then grew under normal conditions for 2 days to harvest seeds.

The above product—T3 transformed ZmNAC111 Arabidopsis strains (TL1-TL3) were extracted. The total RNA of the T3 transformed empty vector Arabidopsis (CK or VC) was obtained and reversely transcribed to obtain cDNA, cDNA served as a template. PCR amplification was performed on cDNA of genes ZmNAC111 with specific primers F1 and R1, wherein the gene of Actin2 of Arabidopsis was an internal reference and the primers were FC and RC.

Electrophoresis results of PCR amplification products are shown in FIG. 15b, wherein all T3 transformed ZmNAC111 Arabidopsis strains can amplify a target fragment 1428 bp and CK (VC) plants did not express the target gene ZmNAC111. This demonstrates that ZmNAC111 can be expressed in T3 transformed ZmNAC111 Arabidopsis and the amount of expression is very high.

Drought Tolerant Phenotypic Analysis of Transformed ZmNAC111 Arabidopsis

Plants of 7-day seedling age of the following were obtained: T3 transformed ZmNAC111 Arabidopsis strains (TL1-TL3 or OE6, OE7, OE8), the wild type Arabidopsis (CK or VC) and T3 transformed empty vector Arabidopsis (CK or VC). These plants were transferred to bowls containing 100 g nutrient soil and were allowed to grow under normal conditions for 25 days. After that, these plants were subjected to a drought treatment (i.e. stopping watering). 14 days later, there were obvious differences on the phenotype of the plants, i.e. CK/VC strain rosette leaves were severely dried up while the rosette leaves of TL1-TL3 strains were heavily wilting, these plants were then re-watered. After re-watering for 6 days, statistics for survival rate of each plant in each strain were obtained (plants which grew normally and could be harvested were defined as survival ones; plants which failed to grow normally and could not be harvested, as well as severely influenced by the drought were defined as dead ones; the survival rate is the percentage of the number of survival plants divided by the total number of plants in each strain). The experiment was repeated for 3 times. In each repeated experiment, there were no less than 30 plants in each strain, and the average value was evaluated for the statistics analysis.

Results are shown in Table 2 and FIG. 15. After drought treatment, the survival rate of T3 transformed ZmNAC111 Arabidopsis strain (TL1-TL3) is higher than that of the wild type Arabidopsis.

The results of T3 transformed empty vector Arabidopsis (CK or VC) have no remarkable difference with that of the wild type Arabidopsis.

TABLE 2
Survival rate (%) results of the transformed ZmNAC111
Arabidopsis after drought treatment.

| Strain | Repetition 1 | Repetition 2 | Repetition 3 | Mean ± S.D. |
|---|---|---|---|---|
| TL1 (OE6) | 100 | 75.00 | 96.88 | 90.62 ± 8.62** |
| TL2 (OE7) | 87.55 | 87.55 | 78.12 | 84.38 ± 5.41** |
| TL3 (OE8) | 75.02 | 90.63 | 90.00 | 85.42 ± 4.02** |
| CK | 25.00 | 33.33 | 25.00 | 27.78 ± 4.81 |

Note:
* denotes statistically significant as p < 0.05, comparing to CK results;
**denotes extremely statistically significant as p < 0.01, comparing to CK results.

ABA-Sensitivity in the Transgenic Arabidopsis.

The VC and 35S:ZmNAC111 transgenic plants were grown in parallel and harvested. Seeds obtained from these plants were planted on ½×MS plates containing 1% sucrose and were supplemented with or without different concentrations of ABA (0, 0.5. and 1 μM ABA). Plates were chilled at 4° C. in the dark for 5 days for stratification and moved to 22° C. with a 16-h-light/8-h-dark cycle. Germination (emergence of radicals) was scored on the 3rd day after germination, with three replicated assays.

Stomatal aperture assays were conducted as previously described [67]. Briefly, rosette leaf peels were floated in a stomatal opening solution (10 mM MES-Tris, pH 6.15, 100 μM CaCl₂), and 10 mM KCl) for 2 hrs and then transferred to a solution supplemented with various concentrations of ABA (0, 0.1, 1, and 10 μM) for another 2 hrs. Subsequently, the abaxial surface of each leaf was applied to 3M clear tape to peel off the epidermal layer. Stomatal apertures were imaged and measured using Image J software. Forty-five stomatal apertures were analyzed in each experiment and the reported values represent the mean±s.d.

The results are shown in FIGS. 15f and e, wherein 15e shows the results on phenotype and 15f shows the statistic results of the degree of stomatal aperture. In the stomatal opening solution of 0 μM ABA, the stomatal aperture of T3 transformed ZmNAC111 Arabidopsis and the wild type Arabidopsis leaves have no remarkable differences. However, as the concentration of ABA increased, there were no obvious changes to the stomatal aperture of wild type plants while the stomatal aperture of T3 transformed ZmNAC111 Arabidopsis remarkably decreased. This result shows, under the induction of ABA, the closing of the stoma in T3 transformed ZmNAC111 Arabidopsis was remarkably faster than that of the wild type plants. There was no obvious difference between T3 transformed empty vector and the wild type arabidopsis. Therefore, ZmNAC111 promotes aperture closure under ABA stress.

Generation and Analysis of the Transgenic Maize.

In summary, the coding region of ZmNac111 was amplified from B73 and the sequence-confirmed PCR fragment was inserted into the pSBII vector under the control of the Zmubi1 promoter. The pSBI plasmid was then inserted into the LBA4404 A. tumefaciens strain. The LBA4404 strain, with the integrated pSBIII plasmid, was then used to deliver the Zmubi1:ZmNAC111 expression cassette into the A188 maize inbred line as described68. Transgenic T0, T1, and T2 plants were grown in a greenhouse under a 16 h-light/8 h-dark condition. Transgenic positive and the sibling transgenic-negative (WT) plants were determined in each generation by PCR analysis for the transgene. The expression of ZmNac111 in transgenic plants was determined by qRT-PCR. Three independent T2 lines, ZmNAC111-OE1, ZmNAC111-OE3, and ZmNAC111-OE7, were selected for further analyses.

In more detail, the recombinant tumefaciens Y transformed corn inbred line A188 by gene transformation method mediated by tumefaciens to obtain T0 generation plants which grew in a greenhouse (16 hours-illumination/6 hours-darkness).

RNA of T2 transformed ZmNAC111 corn plant was extracted and reversely transcribed to obtain cDNA. PCR identification was performed on cDNA of gene ZmNAC111 with the use of specific primers F2 and R2.

The sequences of the above-mentioned primers are as follow:

F2:
(SEQ ID NO. 33)
5′-CTACTATGACGACGACAACT-3′;
R2:
(SEQ ID NO. 34)
5′-CACTCGCTTCCTCTTGTT-3′;

1125 bp positive T0 generation transformed ZmNAC111 corn was obtained.

After self-fertilization of the positive T0 generation transformed ZmNAC111 corn, T1 generation seeds were obtained. After using the same method of applying PCR identification on T1 plants, positive plants were obtained. After self-fertilization, T2 seeds were obtained.

The RNA of T2 transformed ZmNAC111 corn was extracted and reversely transcribed to obtain cDNA. qPCR quantification was performed on cDNA of gene ZmNAC111 with the specific primers F2 and R2, wherein the corn gene Zmubi2 was used as an internal reference and the wild type corn was used as the control.

The sequences of the above-mentioned primers are as follow:

F2:
(SEQ ID NO. 33)
5′-CTACTATGACGACGACAACT-3′;
R2:
(SEQ ID NO. 34)
5′-CACTCGCTTCCTCTTGTT-3′;
FC2:
(SEQ ID NO. 35)
5′-TGGTTGTGGCTTCGTTGGTT-3′;
RC2:
(SEQ ID NO. 36)
5′-GCTGCAGAAGAGTTTTGGGTACA-3′.

The results are as shown in FIG. 5b. The gene expression level of ZmNAC111 in T2 generation ZmNAC111 corn TML1(or OE1), TML2 (OE3) and TML3(OE7) was higher than that of the wild type corn. This shows that T2 generation ZmNAC111 corn TML1, TML2 and TML3 are positive transgenic corns.

The above T0 generation represents plants obtained by transforming the present generation; T1 generation represents seeds produced by the self-fertilization of T0 generation and plants grown from the seeds; T2 generation represents seeds produced by the self-fertilization of T1 generation and plants grown from the seeds.

The specific procedures of genetic transformation method mediated by the above-mentioned tumefaciens are as follows:

The recombinant tumefaciens Y was inoculated in YEB liquid medium containing 25 mg/L spectinomycin and incubated at 28° C. with shaking until its OD600 became 0.5. The corn young embryo was placed in 2 mL centrifuge tube filled with a preserving fluid and subjected to a thermal treatment at 46° C. for 3 minutes, followed by centrifugation at 4° C. and 2000 rpm for 10 minutes. The prepared recombinant tumefaciens was then added to the young embryo after treatment, incubated in darkness at 22° C. for 3 days, and then, transferred to a new medium for incubation at darkness, 28° C. for 7 to 10 days. By screening with phosphinothricin at different concentrations, it was finally transferred to a differential medium and subsequently transferred to a rooting medium for cultivation. Once the plant grew into a certain size, it would be transferred to the soil with nutrients.

The recombinant tumefaciens CK1 transformed corn inbred line A188 with the genetic transformation method mediated by tumefaciens, and was cultivated until T2 generation transformed empty vector corn was obtained.

Transgenic-positive (individually genotyped by transgene-based PCR analysis) and WT plants were planted side-by-side in enriched soil (soil and vermiculite in a ratio of 1:1). Drought treatment was applied to the soil-grown plants at the 3-leaf seedling stage by withholding water. After approximately 20 days, watering was resumed to allow plants to recover. The number of surviving plants was recorded seven days later. At least 15 plants of each line were compared in each test and statistical analyses were based on data obtained from three independent experiments. Stomatal conductance, photosynthetic and transpiration rates of the T2 transgenic and WT plants were measured on fully-expended leaves of seedlings at the 3-leaf stage using a LICOR-6400 CO2 gas exchange analyzer (LICOR-6400, Lincoln, Nebr.). SWC was recorded every other day after the initiation of water withholding. Statistical analysis was based on data obtained from seven seedlings for each plant line and the experiment was repeated twice.

Results are shown in Table 3 and FIGS. 5c and 5d. The dryness of leaves in T2 generation transformed ZmNAC111 corn was less severe than that in the wild type corn. For the survival rate statistics, the survival rate of T2 generation transformed ZmNAC111 corn is higher than that of the wild type corn. There is no remarkable difference between the results of T2 generation transformed empty vector corn and that of the wild type corn.

TABLE 3
Survival rate (%) results of the transformed
ZmNAC111 corn after drought treatment.

| Strain | Repetition 1 | Repetition 2 | Repetition 3 | Mean ± S.D. |
|---|---|---|---|---|
| TML1 (OE1) | 80.00 | 86.67 | 87.64 | 84.78 ± 3.85** |
| TML2 (OE3) | 86.67 | 73.33 | 80.00 | 80.00 ± 6.67** |
| TML3 (OE7) | 80.00 | 83.33 | 82.69 | 75.55 ± 1.70** |
| WT | 74.54 | 77.67 | 74.44 | 28.89 ± 3.85 |

Note:
* denotes statistically significant as p < 0.05, comparing to WT results;
**denotes extremely statistically significant as p < 0.01, comparing to WT results.
Accordingly, ZmNAC111 can improve the drought tolerance of corn.

Photosynthesis Analysis for the Transformed ZmNAC111 Corn

Net photosynthetic rate (Pn), stomatal conductance (Gs) and transpiration rate (Tr) were measured with Li6400 portable photosynthesis system (LICOR-6400, Lincoln, Nebr.). In the third-leaf stage, the third unfolded leaf of T2 generation transformed ZmNAC111 corn strains (TML1-TML3 (OE1, OE3, OE7)) and the wild type corn plants (WT) were measured and subjected to a treatment of no watering, which were then measured once every two days. The corresponding water content in the soil was recorded. 7 plants were randomly measured in each transformation time and the measurement was repeated for 2 times. The measured results are expressed by mean values.

The results are shown in FIG. 5e to h, and are the statistic results of the rate of photosynthesis, stomatal conductance, transpiration rate and water utilization efficiency respectively. Comparing with the wild type corn, the rate of photosynthesis, stomatal conductance and transpiration rate of T2 generation transformed ZmNAC111 corn decreased under the drought treatment, which further increased water utilization efficiency and results in a remarkable increase in survival rate.

RNA-Seq Analysis of Transgenic Maize.

For maize RNA-seq analysis, pooled tissues from three eight-day-old maize seedlings were collected from transgenic and WT plants, prior to or after 2-hour dehydration on a clean bench, to conduct the RNA-seq analysis. Total RNA was isolated using TRIZOL reagent (Biotopped) and RNA integrity was evaluated using a Bioanalyzer 2100 (Agilent). The 100-bp paired-end Illumina sequencing was conducted at Berry Genomics (Beijing). An average of 3 gigabases of raw data were generated for each sample. Differential gene expression was determined using Strand NGS 2.0 software. A total of 31,501 genes were identified, representing ˜79% of all the predicted genes in maize. Enrichment analysis of gene ontology of biological pathways (GOBPs) was performed using the DAVID software program69 (http://david.abcc.ncifcrf.gov/) to compute P-values that indicate the significance of each GOBP being represented by the genes. GOBPs with P<0.01 were identified as enriched processes. qRT-PCR of selected genes that were determined to be critical to drought tolerance was performed to verify the RNA-seq data.

RNA-Seq Analysis of Transformed ZmNAC111 Arabidopsis

20-day aged plants of the following were obtained: the transformed ZmNAC111 Arabidopsis strains (TL1-TL3) and T3 transformed empty vector Arabidopsis strain (CK). They were respectively subjected to drying for 0 hr and 1 hr, wherein each strain had at least 10 seedlings. The total RNA was isolated by TRIZOL (Biotopped) method and the concentration was measured by using Nanodrop1000 (Thermo Scientific product, USA). Then, the total RNA was sent to BerryGenomic Corporation in Beijing to perform transcriptome profiling with a sequencing depth of 3 GB. Lastly, the data was evaluated with Strand NGS 2.0 software

Nucleotide Diversity and Tests for Neutrality.

The genomic region of ZmNac111 was amplified and sequenced in 42 teosinte accessions. Nucleotide diversity (Tr) and the Tajima's D-statistic were calculated using DnaSP version 5.070.

Phylogenetic Tree Construction.

The full-length amino acid sequences of 55 NAC TF encoding genes identified in maize, rice, Arabidopsis, and sorghum were aligned using the Clustal X 1.83 program with default parameters. The phylogenetic tree was constructed based on this alignment result using the neighbor joining (NJ) method in MEGA version 5 with the following parameters: Poisson correction, pairwise deletion, uniform rates and bootstrap (1000 replicates).

Transactivation Activity Assay.

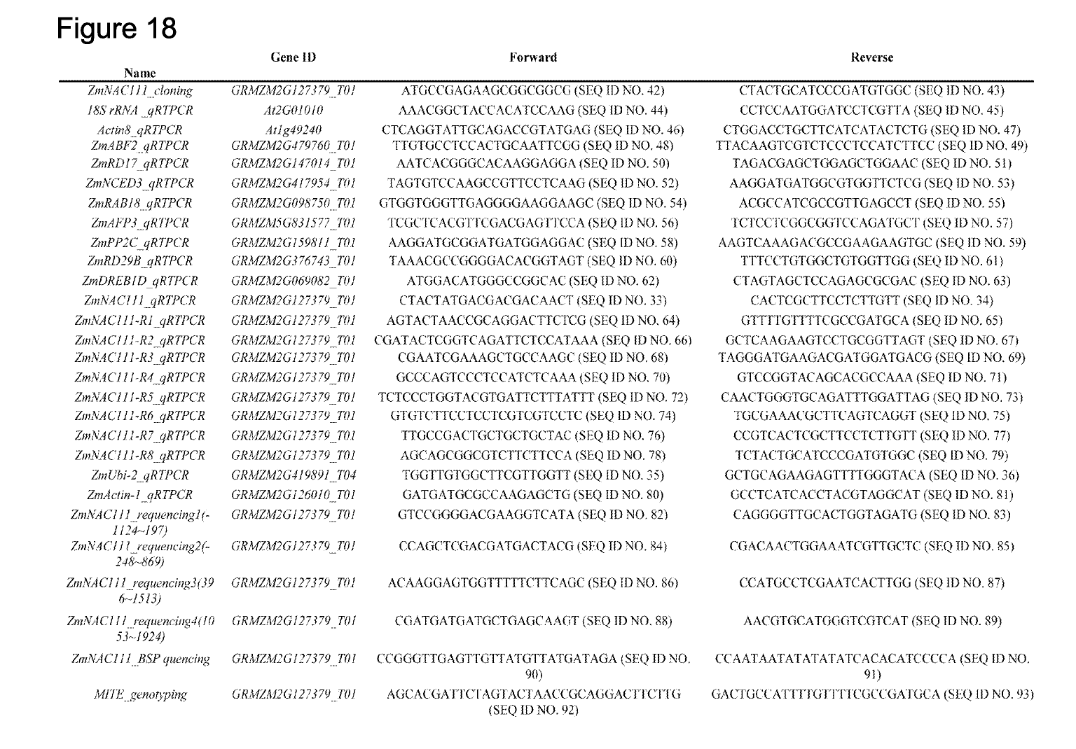

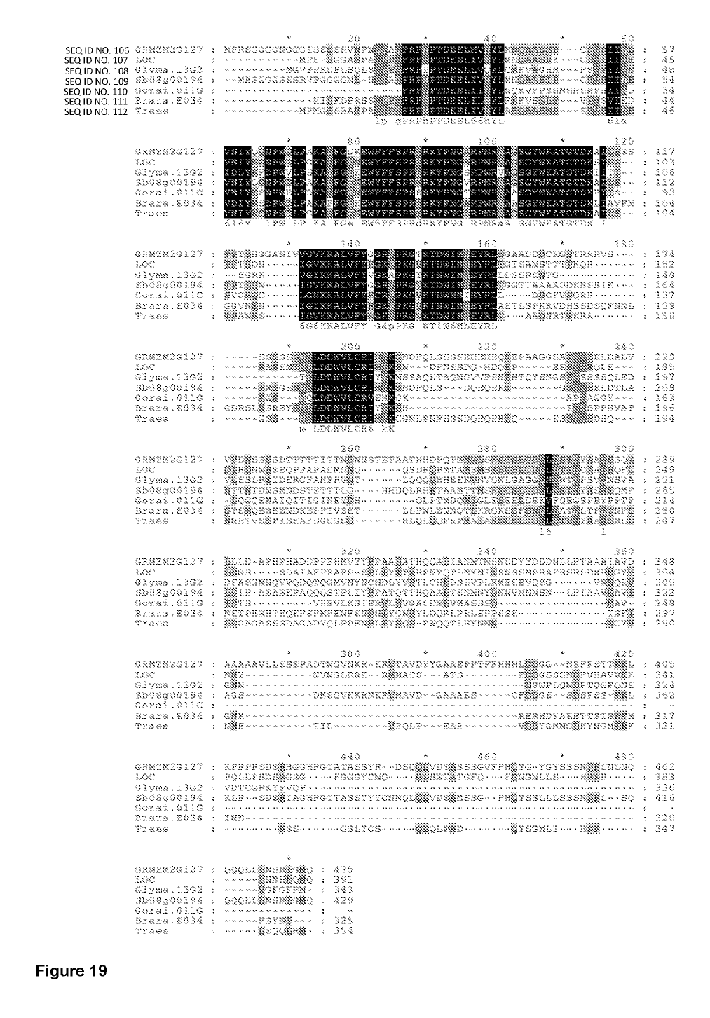

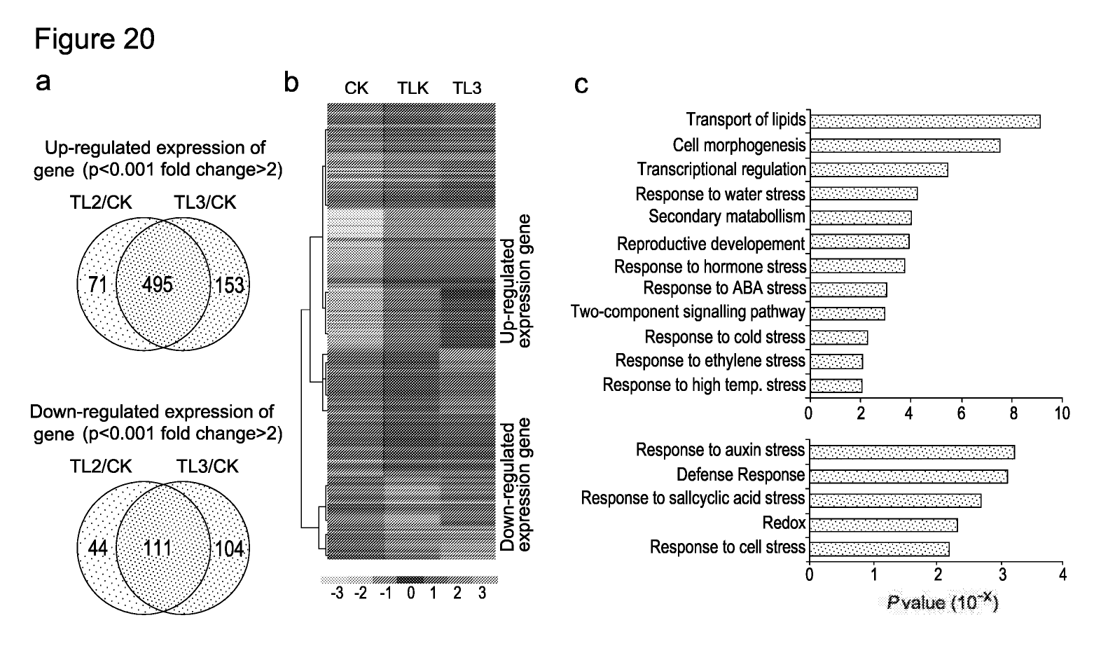

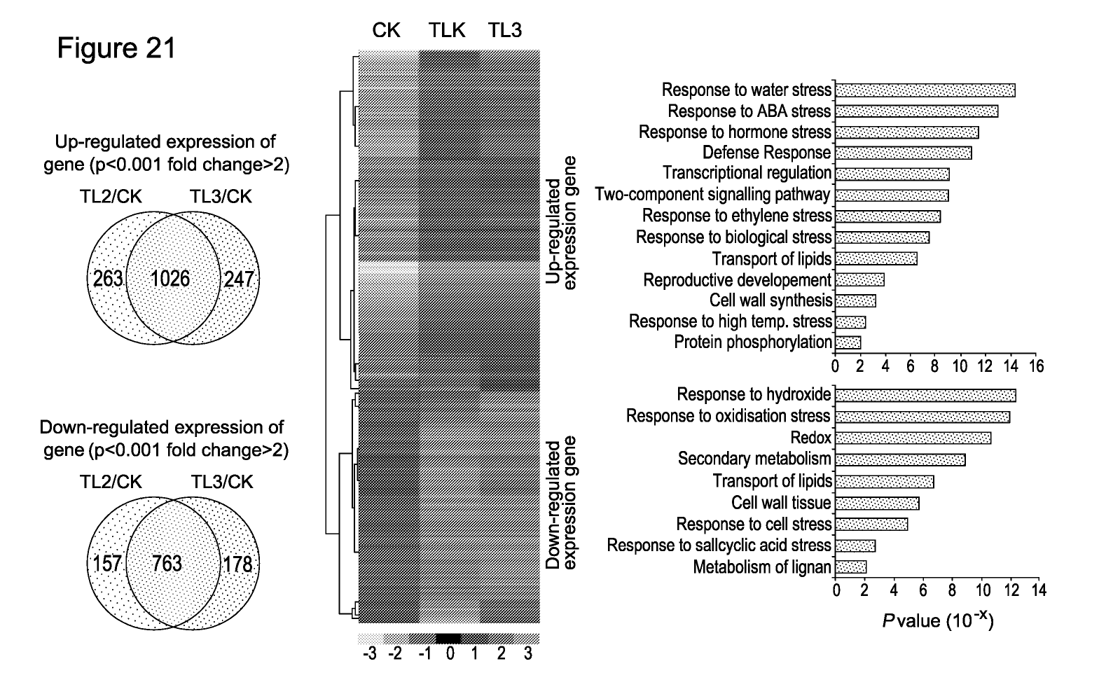

cDNAs of ZmNac111 from ten maize inbred lines were individually cloned into the pGBKT7 vector for evaluating protein transactivation activity in the AH109 yeast strain. The cell concentration of yeast transformants was adjusted to an OD600 of 0.1, and then plated on various selective plates, SD/-T, SD/-T-H, SD/-T-H-A, to compare their survival. Plates were incubated at 30° C. for 2-5 days before photographing. All the primers used in this research are listed in FIG. 18.

### REFERENCES

1. Lobell, D. B. et al. Greater sensitivity to drought accompanies maize yield increase in the U.S. Midwest. Science 344, 516-519 (2014).
2. Pennisi, E. Plant genetics. The blue revolution, drop by drop, gene by gene. Science 320, 171-173 (2008).
3. Duvick, D. N. The contribution of breeding to yield advances, in maize (Zea mays L.). Adv. Agronomy 86, 83-145 (2005).
4. Hao, Z. et al. Meta-analysis of constitutive and adaptive QTL for drought tolerance in maize. Euphytica 174, 165-177 (2010).
5. Semagn, K. et al. Meta-analyses of QTL for grain yield and anthesis silking interval in 18 maize populations evaluated under water-stressed and well-watered environments. BMC Genomics 10, 313-328 (2013).
6. Messmer, R. et al. Drought stress and tropical maize: QTL-by-environment interactions and stability of QTLs across environments for yield components and secondary traits. Theor. Appl. Genet. 119, 913-930 (2009).
7. Almeida, G. D. et al. QTL mapping in three tropical maize populations reveals a set of constitutive and adaptive genomic regions for drought tolerance. Theor. Appl. Genet. 126, 583-600 (2013).
8. Wang, H. et al. The origin of the naked grains of maize. Nature 436, 714-719 (2005).
9. Studer, A., Zhao, Q., Ross-Ibarra, J. & Doebley, J. Identification of a functional transposon insertion in the maize domestication gene tb1. Nat. Genet. 43, 1160-1163 (2011).
10. Salvi, S. et al. Conserved noncoding genomic sequences associated with a flowering time quantitative trait locus in maize. Proc. Natl Acad. Sci. USA 104, 11376-11381 (2007).
11. Hung, H. Y. et al. ZmCCT and the genetic basis of day-length adaptation underlying the postdomestication spread of maize. Proc. Natl Acad. Sci. USA 109, 1913-1921 (2012).
12. Zuo, W. et al. A maize wall-associated kinase confers quantitative resistance to head smut. Nat. Genet. 47, 151-157 (2015).
13. Price, A. H. Believe it or not, QTLs are accurate! Trends Plant Sci. 11, 213-216 (2006).
14. Yu, J. & Buckler, E. S. Genetic association mapping and genome organization of maize. Curr. Opin. Biotechnol. 17, 155-160 (2006).
15. Atwell, S. et al. Genome-wide association study of 107 phenotypes in Arabidopsis thaliana inbred lines. Nature 465, 627-631 (2010).
16. Riedelsheimer, C. et al. Genome-wide association mapping of leaf metabolic profiles for dissecting complex traits in maize. Proc. Natl Acad. Sci. USA 109, 8872-8877 (2012).
17. Huang, X. et al. Genome-wide association study of flowering time and grain yield traits in a worldwide collection of rice germplasm. Nat. Genet. 44, 32-39 (2011).
18. Yan, J. et al. Rare genetic variation at Zea mays crtRB1 increases beta-carotene in maize grain. Nat. Genet. 42, 322-327 (2010).
19. Li, H. et al. Genome-wide association study dissects the genetic architecture of oil biosynthesis in maize kernels. Nat. Genet. 45, 43-50 (2013).
20. Yang, Q. et al. CACTA-like transposable element in ZmCCT attenuated photoperiod sensitivity and accelerated the postdomestication spread of maize. Proc. Natl Acad. Sci. USA 110, 16969-16974 (2013).
21. Xue, Y. et al. Genome-wide association analysis for nine agronomic traits in maize under well-watered and water-stressed conditions. Theor. Appl. Genet. 126, 2587-2596 (2013).
22. Lu, Y. et al. Joint linkage-linkage disequilibrium mapping is a powerful approach to detecting quantitative trait loci underlying drought tolerance in maize. Proc. Natl Acad. Sci. USA 107, 19585-19590 (2010).
23. Setter, T. L. et al. Genetic association mapping identifies single nucleotide polymorphisms in genes that affect abscisic acid levels in maize floral tissues during drought. J. Exp. Bot. 62, 701-716 (2011).
24. Liu, S. et al. Genome-wide analysis of ZmDREB genes and their association with natural variation in drought tolerance at seedling stage of Zea mays L. PLoS Genet. 9, e1003790 (2013).
25. Schnable, P. S. et al. The B73 maize genome: complexity, diversity, and dynamics. Science 326, 1112-1115 (2009).
26. Lisch, D. Regulation of transposable elements in maize. Curr. Opin. Plant Biol. 15, 511-516 (2012).
27. Feschotte, C., Jiang, N. & Wessler, S. R. Plant transposable elements: where genetics meets genomics. Nat. Rev. Genet. 3, 329-341.
28. Makarevitch, I. et al. Transposable elements contribute to activation of maize genes in response to abiotic stress. PLoS Genet. 11, e1004915 (2014).
29. Castelletti, S., Tuberosa, R., Pindo, M. & Salvi, S. A MITE transposon insertion is associated with differential methylation at the maize flowering time QTL Vgt1. G3. 4, 805-812 (2014).
30. Wei, L. et al. Dicer-like 3 produces transposable element-associated 24-nt siRNAs that control agricultural traits in rice. Proc. Natl Acad. Sci. USA 111, 3877-3882 (2014).
31. Law, J. A. & Jacobsen, S. E. Establishing, maintaining and modifying DNA methylation patterns in plants and animals. Nat. Rev. Genet. 11, 204-220 (2010).
32. Zhang, H. & Zhu, J. K. RNA-directed DNA methylation. Curr. Opin. Plant Biol. 14, 142-147 (2011).
33. Stroud, H., Greenberg, M. V., Feng, S., Bernatavichute, Y. V. & Jacobsen, S. E. Comprehensive analysis of silencing mutants reveals complex regulation of the Arabidopsis methylome. Cell 152, 352-364 (2013).
34. Jackson, J. P., Lindroth, A. M., Cao, X. & Jacobsen, S. E. Control of CpNpG DNA methylation by the KRYPTONITE histone H3 methyltransferase. Nature 416, 556-560 (2002).
35. Voitsik, A. M., Muench, S., Deising, H. B. & Voll, L. M. Two recently duplicated maize NAC transcription factor paralogs are induced in response to Colletotrichum graminicola infection. BMC Plant Biol. 13, 85-100 (2013).
36. Jeong, J. S. et al. Root-specific expression of OsNAC10 improves drought tolerance and grain yield in rice under field drought conditions. Plant Physiol. 153, 185-197 (2010).
37. Taoka, K. et al. The NAC domain mediates functional specificity of CUP-SHAPED COTYLEDON proteins. Plant J. 40, 462-473 (2004).
38. Xie, Q., Frugis, G., Colgan, D. & Chua, N. H. Arabidopsis NAC1 transduces auxin signal downstream of TIR1 to promote lateral root development. Genes Dev. 14, 3024-3036 (2000).
39. Mitsuda, N. et al. NAC transcription factors, NST1 and NST3, are key regulators of the formation of secondary walls in woody tissues of Arabidopsis. Plant Cell 19, 270-280 (2007).
40. Yang, S. D., Seo, P. J., Yoon, H. K. & Park, C. M. The Arabidopsis NAC transcription factor VN12 integrates abscisic acid signals into leaf senescence via the COR/RD genes. Plant Cell 23, 2155-2168 (2011).
41. Uauy, C., Distelfeld, A., Fahima, T., Blechl, A. & Dubcovsky, J. A NAC Gene regulating senescence improves grain protein, zinc, and iron content in wheat. Science 314, 1298-1301 (2006).
42. Nakashima, K. et al. NAC transcription factors in plant abiotic stress responses. Biochim. Biophys. Acta. 1819, 97-103 (2012).
43. luchi, S. et al. Regulation of drought tolerance by gene manipulation of 9-cis-epoxycarotenoid dioxygenase, a key enzyme in abscisic acid biosynthesis in Arabidopsis. Plant J. 27, 325-333 (2001).
44. Garcia, M. E., Lynch, T., Peeters, J., Snowden, C. & Finkelstein, R. A small plant-specific protein family of ABI five binding proteins (AFPs) regulates stress response in germinating Arabidopsis seeds and seedlings. Plant Mol. Biol. 67, 643-658 (2008).
45. Nylander, M., Svensson, J., Palva, E. T. & Welin, B. V. Stress-induced accumulation and tissue-specific localization of dehydrins in Arabidopsis thaliana. Plant Mol. Biol. 45, 263-279 (2001).
46. Nakashima, K. et al. Transcriptional regulation of ABI3- and ABA-responsive genes including RD29B and RD29A in seeds, germinating embryos, and seedlings of Arabidopsis. Plant Mol. Biol. 60, 51-68 (2006).
47. Nishimura, N. et al. ABA-Hypersensitive Germination1 encodes a protein phosphatase 2C, an essential component of abscisic acid signaling in Arabidopsis seed. Plant J. 50, 935-949 (2007).
48. Haake, V. et al. Transcription factor CBF4 is a regulator of drought adaptation in Arabidopsis. Plant Physiol. 130, 639-648 (2002).
49. Tran, L. S. et al. Isolation and functional analysis of Arabidopsis stress-inducible NAC transcription factors that bind to a drought-responsive cis-element in the early responsive to dehydration stress 1 promoter. Plant Cell 16, 2481-2498 (2004).
50. Hu, H. et al. Overexpressing a NAM, ATAF, and CUC (NAC) transcription factor enhancesdrought tolerance and salt tolerance in rice. Proc. Natl Acad. Sci. USA 103, 12987-12992 (2006).
51. Yang, X. et al. Genetic analysis and characterization of a new maize association mapping panel for quantitative trait loci dissection. Theor. Appl. Genet. 121, 417-431 (2010).
52. Gent, J. I. et al. CHH islands: De novo DNA methylation in near-gene chromatin regulation in maize. Genome Res. 23, 628-637 (2013).
53. Gent, J. I. et al. Accessible DNA and relative depletion of H3K9me2 at maize loci undergoing RNA-directed DNA methylation. Plant Cell 26, 4903-4917 (2014).
54. Qin, F., Shinozaki, K. & Yamaguchi-Shinozaki, K. Achievements and challenges in understanding plant abiotic stress responses and tolerance. Plant Cell Physiol. 52, 1569-1582 (2011).
55. Tran, L. S., Nishiyama, R., Yamaguchi-Shinozaki, K. & Shinozaki, K. Potential utilization of NAC transcription factors to enhance abiotic stress tolerance in plants by biotechnological approach. GM Crops. 1, 32-39 (2010).
56. Blum, A. Effective use of water (EUW) and not water-use efficiency (VVUE) is the target of crop yield improvement under drought stress. Field Crops Research 112, 119-123 (2009).
57. Lisch, D. How important are transposons for plant evolution? Nat. Rev. Genet. 14, 49-61 (2013).
58. Yang, N. et al. Genome wide association studies using a new nonparametric model reveal the genetic architecture of 17 agronomic traits in an enlarged maize association panel. PLoS Genet. 10, e1004573 (2014).
59. Bradbury, P. J. et al. TASSEL: software for association mapping of complex traits in diverse samples. Bioinformatics 23, 2633-2635 (2007).
60. Price, A. L. et al. Principal components analysis corrects for stratification in genome-wide association studies. Nat. Genet. 38, 904-909 (2006).
61. Fu, J. et al. RNA sequencing reveals the complex regulatory network in the maize kernel. Nat. Commun. 4, 2832 (2013).
62. Purcell, S. et al. PLINK: a tool set for whole-genome association and population-based linkage analyses. Am. J. Hum. Genet. 81, 559-575 (2007).
63. Livak, K. J. & Schmittgen, T. D. Analysis of relative gene expression data using real-time quantitative PCR and the 2^{-ΔΔCT} Method. Methods 25, 402-408 (2001).
64. He, Y., Michaels, S. D. & Amasino, R. M. Regulation of flowering time by histone acetylation in Arabidopsis. Science 302, 1751-1754 (2003).
65. Bowler, C. et al. Chromatin techniques for plant cells. Plant J. 39, 776-789 (2004).
66. Qin, F. et al. Arabidopsis DREB2A-interacting proteins function as RING E3 ligases and negatively regulate plant drought stress-responsive gene expression. Plant Cell 20, 1693-1707 (2008).
67. Ryu, M. Y., Cho, S. K. & Kim, W. T. The Arabidopsis C3H2C3-type RING E3 ubiquitin ligase AtAIRP1 is a positive regulator of an abscisic acid-dependent response to drought stress. Plant Physiol. 154, 1983-1997 (2010).
68. Ishida, Y., Hiei, Y. & Komari, T. Agrobacterium-mediated transformation of maize. Nat. Protoc. 2, 1614-1621 (2007).
69. Huang, D. W., Sherman, B. T. & Lempicki, R. A. Systematic and integrative analysis of large gene lists using DAVID bioinformatics resources. Nat. Protoc. 4, 44-57 (2009).
70. Librado, P. & Rozas, J. DnaSP v5: A software for comprehensive analysis of DNA polymorphism data. Bioinformatics 25, 1451-1452 (2009).
71. Cermak, T. et al. Efficient design and assembly of custom TALEN and other TAL effector-based constructs for DNA targeting. Nucleic Acids Res. 39 (2011).

Sequence Information

SEQ ID NO: 1: ZmNac111 nucleic acid sequence (genomic)
AAAAATTTCAATGCCTCTCTAGATCTTTCAAGCTATCCTTGCTTCCTAGCTCCCTTT
GGCGTGCTGTACCGGACGACAGGGGTAGGTAGGTAGGTAGGTGTAGCTAGCTCG
GCGAGCGAGGGAGCAATCCGGCCAACAGCACACGCGTCGATCGCCATGCCGAG
AAGCGGCGGCGGCGGCAATGGCGGTGGCATTAGTAGTAGTAGTAGGGTGATGCC
CAACCTCCCGGCCGGGTTCCGCTTCCACCCCACGGACGAGGAGCTCATGGTGCA
CTACCTCATGAGGCAGGCCGCCTCCATGCCGTGCCCCGTCCCCATCATCGCCGA
GGTCAACATCTACCAGTGCAACCCCTGGGATCTCCCTGGTACGTGATTCTTTATTT
CTTTCTTCCCTCTCTTATTCGTTCATTGTCACTTGAGTTGAGTCGTTCAGACTAGTT
GTGTTATTCCTAGCTAATCCAAATCTGCACCCAGTTGAATCTCATATGATTTAATTA
TTCTTTCTTCCTCCTGTGGTATCGTGTTCGTTCTTCAGCCAAGGCATTGTTCGGCG
ACAAGGAGTGGTTTTTCTTCAGCCCCCGGGACCGCAAGTACCCCAACGGCGCCC
GCCCCAACCGCGCCGCCGGGTCCGGGTACTGGAAGGCCACCGGCACCGACAAG
GCCATCCTGTCGTCGTCCACGCCCACGAGCCACGGCGGCGCCAACATCGTCGTC
GGCGTCAAGAAGGCGCTCGTCTTCTACGGCGGCAGGCCGCCCAAGGGCACCAA
GACGGACTGGATCATGCACGAGTACCGCCTCTCGGGCGCGGCGGACGACGACT
GCAAGGGCAGCACCAGGCGCAGAGTGTCTTCCTCCTCGTCGTCCTCCATGAGGG
TGAGTGAGACTTTCTTTTTCTTTTTCTTCTTTTTTTTTTAATTGCACTTGCTTGCACG
GCTCCAGACCTGACTGAAGCGTTTCGCATGGTTTCAGCTGGACGACTGGGTGCT
GTGCAGGATCCACAAGAAGAGCAACGATTTCCAGTTGTCGTCGTCGTCGGAGCAT
GAGCACGAGCAGGAGGAGCCGGCGGCGGGCGGCTCAGCGACGACTGTGGAGG
AACTAGACGCTCTCGTCGTCGTCGACAACAGCAGCAGCAGCGACACCACCACCA
CCACCATCACCACCAACGACAACAACTCTACTGAAACAGCTGCGACTCATCACGA
TCCTCAGACGATGATGATGCTGAGCAAGTCGTGCTCGCTCACCGACCTCCTCGAC
AGCATCGACTACGCGGCGCTCTCCTCGCAGATGCTCCTCCTCGACGCGCCCCAC
CCCCACGCTGATGACCCGCCGCCACACATGGTCTACTACCCACCGGCGGCGTCG
GCGACGCACCAGCAAGCAATAATAGCTAATAACACCAACAGTAATGACGACTACT
ATGACGACGACAACTTGTTGCCGACTGCTGCTGCTACTGCGGTAGACGCGGCGG
CGGCGGCGGTGCTACTCAGCTCGTCGCCTGCAGACACCAACGGTGTGAACAAGA
GGAAGCGAGTGACGGCCGTGGACTACTACGGCGCCGCTGAACCGCCGACCTTCT
TCCACCACCACCTCGATGATGGCGGCAACAGCTTCTTCAGTACGACCAAGAAGCT
CAAGCCGCCGCCGCCAAGTGATTCGAGGCATGGCGGCCATTTCGGCACCGCCAC
AGCGAGCAGCTACCGCGACAGCCAGCAGCTCGTGGACAGCGCGAGCAGCAGCG
GCGTCTTCTTCCATCAGTATGGGTACGGCTACAGCAGCAGCAACCCGTTCTTGAA
CTTGAACCAGCAGCAGCAGCTGCTACTCAACAGCCACATCGGGATGCAGTAGAG
CTCGAGAGATCGAGAGAGAGAGAGAGAGAGAGATGATATATCATTGGAAGGATCA
GCACGCGTACGTGCAGATTAATTGCAGCGAGTTCTATCTTGTTTGCTTTCATTATT
ATATACTCCGTATGTGTCGTTCATGTAAAATTAGTTTAAATTGTAAATTAGTGTGCT
GAAATTCATGTGCCTGCTCTCTGCTCATCAATGAATGGATGATGACGACCCATGCA
CGTTGCTTGTGTGG
SEQ ID NO: 2: GRMZM2G127379 (ZmNAC111); nucleic acid sequence (coding
sequence)
ATGCCGAGAAGCGGCGGCGGCGGCAATGGCGGTGGCATTAGTAGTAGTAGTAG
GGTGATGCCCAACCTCCCGGCCGGGTTCCGCTTCCACCCCACGGACGAGGAGCT
CATGGTGCACTACCTCATGAGGCAGGCCGCCTCCATGCCGTGCCCCGTCCCCAT
CATCGCCGAGGTCAACATCTACCAGTGCAACCCCTGGGATCTCCCTGCCAAGGC
ATTGTTCGGCGACAAGGAGTGGTTTTTCTTCAGCCCCCGGGACCGCAAGTACCCC
AACGGCGCCCGCCCCAACCGCGCCGCCGGGTCCGGGTACTGGAAGGCCACCGG
CACCGACAAGGCCATCCTGTCGTCGTCCACGCCCACGAGCCACGGCGGCGCCAA
CATCGTCGTCGGCGTCAAGAAGGCGCTCGTCTTCTACGGCGGCAGGCCGCCCAA
GGGCACCAAGACGGACTGGATCATGCACGAGTACCGCCTCTCGGGCGCGGCGG
ACGACGACTGCAAGGGCAGCACCAGGCGCAGAGTGTCTTCCTCCTCGTCGTCCT
CCATGAGGCTGGACGACTGGGTGCTGTGCAGGATCCACAAGAAGAGCAACGATT
TCCAGTTGTCGTCGTCGTCGGAGCATGAGCACGAGCAGGAGGAGCCGGCGGCG
GGCGGCTCAGCGACGACTGTGGAGGAACTAGACGCTCTCGTCGTCGTCGACAAC
AGCAGCAGCAGCGACACCACCACCACCACCATCACCACCAACGACAACAACTCTA
CTGAAACAGCTGCGACTCATCACGATCCTCAGACGATGATGATGCTGAGCAAGTC
GTGCTCGCTCACCGACCTCCTCGACAGCATCGACTACGCGGCGCTCTCCTCGCA
GATGCTCCTCCTCGACGCGCCCCACCCCCACGCTGATGACCCGCCGCCACACAT
GGTCTACTACCCACCGGCGGCGTCGGCGACGCACCAGCAAGCAATAATAGCTAA
TAACACCAACAGTAATGACGACTACTATGACGACGACAACTTGTTGCCGACTGCT
GCTGCTACTGCGGTAGACGCGGCGGCGGCGGCGGTGCTACTCAGCTCGTCGCC
TGCAGACACCAACGGTGTGAACAAGAGGAAGCGAGTGACGGCCGTGGACTACTA
CGGCGCCGCTGAACCGCCGACCTTCTTCCACCACCACCTCGATGATGGCGGCAA
CAGCTTCTTCAGTACGACCAAGAAGCTCAAGCCGCCGCCGCCAAGTGATTCGAG
GCATGGCGGCCATTTCGGCACCGCCACAGCGAGCAGCTACCGCGACAGCCAGC
AGCTCGTGGACAGCGCGAGCAGCAGCGGCGTCTTCTTCCATCAGTATGGGTACG
GCTACAGCAGCAGCAACCCGTTCTTGAACTTGAACCAGCAGCAGCAGCTGCTACT
CAACAGCCACATCGGGATGCAGTAG
SEQ ID NO: 3: ZmNac111 nucleic acid sequence (cDNA)
AAAAATTTCAATGCCTCTCTAGATCTTTCAAGCTATCCTTGCTTCCTAGCTCCCTTT
GGCGTGCTGTACCGGACGACAGGGGTAGGTAGGTAGGTAGGTG
TAGCTAGCTCGGCGAGCGAGGGAGCAATCCGGCCAACAGCACACGCGTCGATCG
CCATGCCGAGAAGCGGCGGCGGCGGCAATGGCGGTGGCATTAGTAG
TAGTAGTAGGGTGATGCCCAACCTCCCGGCCGGGTTCCGCTTCCACCCCACGGA
CGAGGAGCTCATGGTGCACTACCTCATGAGGCAGGCCGCCTCCATG
CCGTGCCCCGTCCCCATCATCGCCGAGGTCAACATCTACCAGTGCAACCCCTGG
GATCTCCCTGCCAAGGCATTGTTCGGCGACAAGGAGTGGTTTTTCT
TCAGCCCCCGGGACCGCAAGTACCCCAACGGCGCCCGCCCCAACCGCGCCGCC
GGGTCCGGGTACTGGAAGGCCACCGGCACCGACAAGGCCATCCTGTC
GTCGTCCACGCCCACGAGCCACGGCGGCGCCAACATCGTCGTCGGCGTCAAGAA
GGCGCTCGTCTTCTACGGCGGCAGGCCGCCCAAGGGCACCAAGACG
GACTGGATCATGCACGAGTACCGCCTCTCGGGCGCGGCGGACGACGACTGCAA
GGGCAGCACCAGGCGCAGAGTGTCTTCCTCCTCGTCGTCCTCCATGA
GGCTGGACGACTGGGTGCTGTGCAGGATCCACAAGAAGAGCAACGATTTCCAGT
TGTCGTCGTCGTCGGAGCATGAGCACGAGCAGGAGGAGCCGGCGGC
GGGCGGCTCAGCGACGACTGTGGAGGAACTAGACGCTCTCGTCGTCGTCGACAA
CAGCAGCAGCAGCGACACCACCACCACCACCATCACCACCAACGAC
AACAACTCTACTGAAACAGCTGCGACTCATCACGATCCTCAGACGATGATGATGC
TGAGCAAGTCGTGCTCGCTCACCGACCTCCTCGACAGCATCGACT
ACGCGGCGCTCTCCTCGCAGATGCTCCTCCTCGACGCGCCCCACCCCCACGCTG
ATGACCCGCCGCCACACATGGTCTACTACCCACCGGCGGCGTCGGC
GACGCACCAGCAAGCAATAATAGCTAATAACACCAACAGTAATGACGACTACTATG
ACGACGACAACTTGTTGCCGACTGCTGCTGCTACTGCGGTAGAC
GCGGCGGCGGCGGCGGTGCTACTCAGCTCGTCGCCTGCAGACACCAACGGTGT
GAACAAGAGGAAGCGAGTGACGGCCGTGGACTACTACGGCGCCGCTG
AACCGCCGACCTTCTTCCACCACCACCTCGATGATGGCGGCAACAGCTTCTTCAG
TACGACCAAGAAGCTCAAGCCGCCGCCGCCAAGTGATTCGAGGCA
TGGCGGCCATTTCGGCACCGCCACAGCGAGCAGCTACCGCGACAGCCAGCAGCT
CGTGGACAGCGCGAGCAGCAGCGGCGTCTTCTTCCATCAGTATGGG
TACGGCTACAGCAGCAGCAACCCGTTCTTGAACTTGAACCAGCAGCAGCAGCTGC
TACTCAACAGCCACATCGGGATGCAGTAGAGCTCGAGAGATCGAG
AGAGAGAGAGAGAGAGAGATGATATATCATTGGAAGGATCAGCACGCGTACGTG
CAGATTAATTGCAGCGAGTTCTATCTTGTTTGCTTTCATTATTATA
TACTCCGTATGTGTCGTTCATGTAAAATTAGTTTAAATTGTAAATTAGTGTGCTGAA
ATTCATGTGCCTGCTCTCTGCTCATCAATGAATGGATGATGAC
GACCCATGCACGTTGCTTGTGTGG
SEQ ID NO: 4 GRMZM2G127379 (ZmNAC111); protein
MPRSGGGGNGGGISSSSRVMPNLPAGFRFHPTDEELMVHYLMRQAASMPCPVPIIA
EVNIYQCNPWDLPAKALFGDKEWFFFSPRDRKYPNGARPNRAAGSGYWKATGTDKA
ILSSSTPTSHGGANIVVGVKKALVFYGGRPPKGTKTDWIMHEYRLSGAADDDCKGST
RRRVSSSSSSSMRLDDWVLCRIHKKSNDFQLSSSSEHEHEQEEPAAGGSATTVEEL
DALVVVDNSSSSDTTTTTITTNDNNSTETAATHHDPQTMMMLSKSCSLTDLLDSIDYA
ALSSQMLLLDAPHPHADDPPPHMVYYPPAASATHQQAIIANNTNSNDDYYDDDNLLP
TAAATAVDAAAAAVLLSSSPADTNGVNKRKRVTAVDYYGAAEPPTFFHHHLDDGGNS
FFSTTKKLKPPPPSDSRHGGHFGTATASSYRDSQQLVDSASSSGVFFHQYGYGYSS
SNPFLNLNQQQQLLLNSHIGMQ
SEQ ID NO: 5 Oryza sativa; LOC_Os11g03300
ATGCCGAGCAGCGGCGGCGCCATGCCTGCCCTTCCACCAGGCTTCCGCTTCCAC
CCCACCGACGAGGAGCTCATCGTTCACTACCTCATGAACCAGGCCGCCTCCGTC
AAGTGCCCCGTGCCAATCATCGCCGAGGTCAACATCTACAAGTGCAACCCATGGG
ACCTTCCTGGTAAGGCTTTGTTCGGCGAGAACGAATGGTACTTCTTCAGCCCGAG
GGACCGCAAGTACCCCAACGGCGCTCGCCCCAACCGCGCCGCCGGCTCGGGGT
ACTGGAAGGCCACCGGCACCGACAAGTCCATCCTCTCCACTCCGACCAGCGACA
ACATCGGCGTCAAGAAGGCCCTCGTCTTCTACAAGGGCAAGCCTCCCAAGGGCG
TCAAGACCGACTGGATCATGCACGAGTACCGTCTCACCGGCACATCAGCTAACAG
CACCACCACCACAAAGCAGCGTAGAGCGTCATCCATGACCATGAGGCTGGACGA
CTGGGTGCTGTGCAGAATCCACAAGAAGAGCAACGACTTCAATTCCTCTGACCAA
CACGACCAAGAACCCGAGGAATCAACCGTCGAACAGCTTGAAGACATCCATGACA
ACAACTCCTCTGAACAACCTCCAGCTCCAGCTGACATGAACAACCAACAGTCAGA
TTTCCAGCCCATGACGGCGATGAGCATGAGCAAGTCATGCTCCCTCACCGATCTC
CTCAACACCATCGACTGCGCCGCGCTCTCGCAGTTTCTCCTCGACGGCTCATCCG
ACGCCATCGCTGAGCCTCCTGCTCCTCCCAGCCCCCTAATATACACAACACCTCA
TCCAAATTACCAAACACTAAACTATAACATTAACAGCAACAGCAGCATGCCACACG
CCTTCGAGTCACGCCTAGATCATCACGATGGTTACGTTAACAATTATAATGTTAAT
GGCCTGAGGAGGAAGAGAATGATGGCGTGTAGTGCAACTTCCTTTGATGATGGCA
GCAGCAGCAATGACTTTGTGCATGCCGTTGTCAAGAAACCGCAGCTGCTGCCAAG
TGATTCGAGGGGTAGTGGTTTTGGAGGAGGTTACTGCAACCAGCAGCTTTCAGAG
ACTGCGACTGGCTTTCAGTTTCAGAACGGCAATCTGCTGAGCCATCCATTTCCTCT
GAACAATCATCTGCAGATGCAGTAG
SEQ ID NO: 6; Oryza sativa; LOC_Os11g03300
MPSSGGAMPALPPGFRFHPTDEELIVHYLMNQAASVKCPVPIIAEVNIYKCNPWDLPG
KALFGENEWYFFSPRDRKYPNGARPNRAAGSGYWKATGTDKSILSTPTSDNIGVKKA
LVFYKGKPPKGVKTDWIMHEYRLTGTSANSTTTTKQRRASSMTMRLDDWVLCRIHKK
SNDFNSSDQHDQEPEESTVEQLEDIHDNNSSEQPPAPADMNNQQSDFQPMTAMSM
SKSCSLTDLLNTIDCAALSQFLLDGSSDAIAEPPAPPSPLIYTTPHPNYQTLNYNINSNS
SMPHAFESRLDHHDGYVNNYNVNGLRRKRMMACSATSFDDGSSSNDFVHAVVKKP
QLLPSDSRGSGFGGGYCNQQLSETATGFQFQNGNLLSHPFPLNNHLQMQ
SEQ ID NO: 7: Glycine max; Glyma.13G279900
ATGGGAGTTCCAGAGAAAGACCCTCTTTCCCAATTGAGTTTGCCTCCTGGTTTTCG
GTTTTACCCCACCGACGAGGAGCTTCTCGTTCAGTATCTGTGCC
GCAAGGTCGCTGGCCACCATTTCTCTCTTCCAATCATTGCCGAAATTGACTTGTAC
AAGTTCGACCCATGGGTTCTTCCAAGTAAAGCGATTTTCGGTGA
AAAAGAGTGGTACTTTTTCAGCCCCAGAGACAGGAAATACCCGAACGGGTCTCGA
CCCAACAGAGTAGCTGGGTCGGGTTATTGGAAAGCCACCGGAACC
GACAAGATCATCACCACCGAAGGTAGAAAAGTTGGCATAAAAAAAGCCCTCGTTTT
CTACGTTGGCAAAGCCCCCAAGGGCACCAAAACCAATTGGATCA
TGCACGAGTATCGCCTCCTCGACTCTTCCCGAAAGAACACTGGCACCAAGCTTGA
TGATTGGGTTCTGTGTCGTATATACAAGAAGAACTCGAGTGCACA
GAAGACGGCGCAAAACGGCGTGGTTCCGAGCAACGAGCACACTCAATACAGCAA
CGGTTCCTCTTCTTCTTCTTCGTCCCAGCTGGAGGACGTTCTGGAA
TCTCTGCCATCGATTGATGAAAGGTGTTTCGCGATGCCACGCGTCAACACGCTGC
AACAACAACAGCACCACGAGGAGAAGGTCAATGTTCAGAACTTGG
GTGCAGGGGGTTTAATGGATTGGACCAACCCTTCGGTTCTGAATTCGGTCGCCGA
TTTCGCTTCGGGGAATAATCAAGTGGTACAGGACCAGACTCAGGG
GATGGTGAACTACAACTGCAATGACCTTTATGTCCCTACGTTATGCCACTTGGACT
CATCGGTTCCGTTAAAGATGGAGGAAGAGGTGCAAAGCGGCGTG
AGAAACCAACGGGTCGGGAATAATAATTCGTGGTTTCTTCAGAATGATTTCACACA
GGGGTTTCAGAATTCGGTTGACACGTGTGGGTTTAAATACCCGG
TTCAGCCGGTCGGGTTCGGGTTCAGAAATTGA
SEQ ID NO: 8: Glycine max; Glyma.13G279900
MGVPEKDPLSQLSLPPGFRFYPTDEELLVQYLCRKVAGHHFSLPIIAEIDLYKFDPWVL
PSKAIFGEKEWYFFSPRDRKYPNGSRPNRVAGSGYWKATGTDKIITTEGRKVGIKKAL
VFYVGKAPKGTKTNWIMHEYRLLDSSRKNTGTKLDDWVLCRIYKKNSSAQKTAQNGV
VPSNEHTQYSNGSSSSSSSQLEDVLESLPSIDERCFAMPRVNTLQQQQHHEEKVNV
QNLGAGGLMDWTNPSVLNSVADFASGNNQVVQDQTQGMVNYNCNDLYVPTLCHLD
SSVPLKMEEEVQSGVRNQRVGNNNSWFLQNDFTQGFQNSVDTCGFKYPVQPVGFG
FRN
SEQ ID NO: 9: Sorghum bicolor; Sb08g001940
ATGGCGAGTGGTGGCGGCAGTAGCAGTAGAGTGCCCGGCGGCGGTGGCAACCT
GAACCTCCCAGCTGGGTTCCGGTTCCACCCCACTGACGAGGAGCTGATCGTGCA
CTACCTCATGAACCAGGCCGCCTCCATTCCGTGCCCTGTCCCCATCATCGCCGAG
GTCAACATCTACCAGTGCAACCCCTGGGATCTCCCTGCAAAAGCATTGTTTGGCG
AGAACGAGTGGTACTTCTTCAGCCCACGAGACCGCAAGTACCCCAATGGCGTCC
GCCCCAACCGCGCCGCCGGGTCAGGGTACTGGAAGGCCACCGGCACCGACAAG
GCCATCCTGTCCACGCCGACGAGCCAGAACATCGGCGTCAAGAAGGCCCTCGTC
TTCTACGGCGGCAGGCCTCCCAAGGGCGTCAAGACCGACTGGATCATGCACGAG
TACCGCCTCACCGGCGGCACGACCGCCGCAGCGGCCGATGACAAGAACAGCAG
CATCAAACGCAGATCAGGGTCCTCCATGAGGCTGGACGACTGGGTGCTGTGCAG
GATCCACAAGAAGAGCAACGATTTCCAGTTGTCGGACCAGGAGCAGGAGAAGGA
GGGCTCAACCGTGGAGGAATTAGACACTCTTGCCAACACCACCAACACCGACAAC
AGCATGAACGACTCTACTGAAACAACAACTCTTGGTCATCATGACCAGCTCCGTCA
TGAAACGGCGGCCATGACGACGATGAGCAAGTCGTGCTCGCTCACCGACCTCCT
CAACAGCATCGACTACGCGTCGCTCTCGCAGATGTTCCTTGACATCCCTGCCGAG
GCCGAGGAGCCTGCACAACAGCAAAGCACTCCACTAATCTACCCACCAGCAACAC
AAACAACACACCAAGCAGCACTTACTAGTAATAATAACTATGACAACAACGTGATG
AACAACAGCAACTTGCCGATTGCTGCAGTAGACGCAGTGATAGCAGGCTCGGATA
ACAGCGGTGTGAAGAAGAGGAATAAGAGAGTGATGGCCGTGGACGGCGCCGCC
GCTGAATCGTGCTTCGATGATGGCAGCAGCGACAGTTTCAGTAGCAAGAAACTGA
AGCTGCCAAGTGATTCGAGGATCGCCGGCCATTTCGGCACCACAGCAAGCAGCT
ACTACTACTGCAACAACCAGCTGCAGCTTGTGGACAGCGCGATGAGTAGTGGCTT
TCATCAGTACAGCAGCCTGTTGCTGAGCAGCAGCAATCCGTTCTTGAGCCAGCAG
CAGCAGCTGCTACTCAACAGCCACATCGGGATGCAGTAG
SEQ ID NO: 10: Sorghum bicolor; Sb08g001940
MASGGGSSSRVPGGGGNLNLPAGFRFHPTDEELIVHYLMNQAASIPCPVPIIAEVNIY
QCNPWDLPAKALFGENEWYFFSPRDRKYPNGVRPNRAAGSGYWKATGTDKAILSTP
TSQNIGVKKALVFYGGRPPKGVKTDWIMHEYRLTGGTTAAAADDKNSSIKRRSGSSM
RLDDWVLCRIHKKSNDFQLSDQEQEKEGSTVEELDTLANTTNTDNSMNDSTETTTLG
HHDQLRHETAAMTTMSKSCSLTDLLNSIDYASLSQMFLDIPAEAEEPAQQQSTPLIYP
PATQTTHQAALTSNNNYDNNVMNNSNLPIAAVDAVIAGSDNSGVKKRNKRVMAVDGA
AAESCFDDGSSDSFSSKKLKLPSDSRIAGHFGTTASSYYYCNNQLQLVDSAMSSGFH
QYSSLLLSSSNPFLSQQQQLLLNSHIGMQ
SEQ ID NO: 11: Gossypium raimondii; Gorai.011G030600
TTCAGATTCCATCCTACTGATGAAGAGCTTATCATTCATTATCTAAACCAGAAGGTT
TTCCCTTCCTCCAATCATCACCTAATGTTCTCCATTATAGCTGATGTTAATATCTAC
AAGTTCAATCCATGGGAACTTCCTGATAAGGCTTTATTTGGTGAGAACGAGTGGTT
TTTCTTTAGTCCAAGAGAAAGGAAGTATCCAAACGGAACACGCCCGAACCGAGCA
GCGGCATCGGGATACTGGAAGGCTACCGGGACCGATAAACCGATCATCGCTTCT
GTTGGTTCACAATGCCTTGGAATGAAGAAAGCTTTAGTGTTTTACAAAGGACGTCC
TCCTAAAGGTGTAAAAACCGATTGGATGATGATTGAATATAGACTGCTCGATGATT
GCTTTGTATCTCAAAGACCTAAAGGATCAATGCAATTGGATGATTGGGTTCTATGC
CGAGTTAGTCACAAAGGCAAAGCTCCATTGGCTGGTGGGTACTTACAAGGTCAAG
AGATGGCAATACAAATCACCATTGGAATCAATGAATATGATCATCAGTTACCTACA
ATGGACCAGATGATTGGCCTTGAAAGTGAAGAGCTCGATGAAAAGGTGTTTCAAG
AAGGGTCCCCAGAGTATCCTCCAACACCCATAGATACCAGTGTGAGAGAGGTGCT
AAAGTCCATTGAAAGGGTACTGTCTGTTGGAGCTTTGGATGAACTGGTAATGGCTT
CTTCAAGTGACGATGCTGTGTGA
SEQ ID NO: 12: Gossypium raimondii; Gorai.011G030600
FRFHPTDEELIIHYLNQKVFPSSNHHLMFSIIADVNIYKFNPWELPDKALFGENEWFFFS
PRERKYPNGTRPNRAAASGYWKATGTDKPIIASVGSQCLGMKKALVFYKGRPPKGVK
TDWMMIEYRLLDDCFVSQRPKGSMQLDDWVLCRVSHKGKAPLAGGYLQGQEMAIQI
TIGINEYDHQLPTMDQMIGLESEELDEKVFQEGSPEYPPTPIDTSVREVLKSIERVLSV
GALDELVMASSSDDAV
SEQ ID NO: 13: Brassica rapa; Brara.E03468
ATGATAAGCAAGGATCCAAGATCAAGTTTGCCACCAGGGTTTCGATTTCATCCAAC
TGATGAAGAACTCATTCTCCATTACCTAAGGAAGAAGGTTTCCTCTTTACCAGTCC
CGCTTTCGGTCATCGAAGATGTCGATATCTATAAATCTGATCCATGGGATTTACCA
GCTAAGGCTCCCTTTGGAGAGAAAGAATGGTATTTTTTCAGTCCGAGGGACAGGA
AATATCCAAACGGAGCAAGACCAAACCGAGCAGCTGCGTCAGGGTATTGGAAAG
CCACCGGAACAGATAAATTAATTGCGGTACCAAACGGTGGAGTTAATGAAAACATT
GGTATAAAAAAAGCTCTTGTGTTTTATAGAGGAAAGCCTCCAAAAGGTGTTAAAAC
CAATTGGATCATGCATGAATATCGACTTGCTGAAACCCTATCGCCCAAAAGAGTG
GACCATTCGAGCGACAGCCAATTCAATAATCTTGGAGACAGGAGTTTGAAATCTA
GAGAATACTCTATGAGGCTGGATGATTGGGTTCTTTGCCGGATTTACAAGAAATCA
CACATTTCACTGTCACCACCACATGTTGCTACTGATACAAGCAACCAAGAACATGA
GGAAAATGACAAAGAACCATTCATAGTCAGCGAAACCCTCTTGCCAAATTTGGAAA
ACAATCAAACACTTAAACGCCAGAAGTCTTCTTTCTCGAACTTATTAGACGCTACA
GATTTGACGTTCCTCACAAACTTTCTAAACGAAACTCCGGAACATCATACTGAACA
AGAGTTTTCTTTCATGTTTGAAAATTTCTCAAACCCTAACATCTACGGAAACCCTTA
CTTGGATCAAAAGTTACCTCGGTTAAGCCCTCCTAGTTCTGAGACCAGCTTTATCG
GAAACAAAAGAGAAAGAATGGATTATGCAGAAGAAACGACGAGCACTTCCAAGAA
GATGATCAACAACTTTAGTTACAATATATAA
SEQ ID NO: 14: Brassica rapa; Brara.E03468
MISKDPRSSLPPGFRFHPTDEELILHYLRKKVSSLPVPLSVIEDVDIYKSDPWDLPAKA
PFGEKEWYFFSPRDRKYPNGARPNRAAASGYWKATGTDKLIAVPNGGVNENIGIKKA
LVFYRGKPPKGVKTNWIMHEYRLAETLSPKRVDHSSDSQFNNLGDRSLKSREYSMRL
DDWVLCRIYKKSHISLSPPHVATDTSNQEHEENDKEPFIVSETLLPNLENNQTLKRQK
SSFSNLLDATDLTFLTNFLNETPEHHTEQEFSFMFENFSNPNIYGNPYLDQKLPRLSPP
SSETSFIGNKRERMDYAEETTSTSKKMINNFSYNI
SEQ ID NO: 15: Triticum aestivum; Traes_5BL_CC18CAD72
ATGCCAATGGGCAGCAGCGCCGCCATGCCCGCCCTCCCTCCCGGCTTCCGGTTC
CACCCCACCGACGAGGAGCTCATCGTCCACTACCTCCGCAGGCAGGCCGCGTCC
ATGCCCAGCCCCGTGCCCATCATCGCCGAGGTCAACATCTACAAGTGCAACCCAT
GGGACCTCCCCGGCAAAGCTTTGTTCGGGGAGAATGAGTGGTACTTCTTCAGCC
CCCGGGATCGCAAGTACCCCAACGGCGCGCGCCCGAACCGCGCCGCCGGGTCC
GGCTACTGGAAGGCCACCGGCACCGACAAGGCCATCCTGTCCACGCCGGCCAAC
GAGAGCATCGGGGTCAAGAAGGCGCTCGTGTTCTACAGGGGCAAGCCGCCCAAG
GGCGTCAAGACCGACTGGATCATGCACGAGTACCGCCTCACCGCAGCCGACAAC
CGGACCACCAAGCGCAGAGGATCCTCCATGAGGCTGGATGACTGGGTGCTGTGT
AGGATCCACAAGAAGTGCGGCAACTTGCCCAACTTCTCCTCCTCTGACCAGGAAC
AGGAGCATGAGCAGGAGAGCTCCACCGTGGAGGACTCGCAGAACAACCACACCG
TGTCGTCGCCCAAGTCCGAGGCCTTCGACGGCGACGGCGACGACCACCTCCAGT
TGCAGCAGTTCCGCCCCATGGCGATCGCCAAGTCGTGCTCCCTCACCGACCTGC
TCAACACCGTCGACTACGCCGCGCTCTCGCACCTCCTCCTCGACGGCGCCGGCG
CCTCGTCGTCGGACGCCGGAGCAGACTACCAGCTGCCTCCCGAAAACCCACTCA
TCTACTCGCAGCCTCCATGGCAACAAACGCTACACTATAATAACAACAACGGCTAC
GTGAACAACGAGACCATCGACGTGCCTCAGCTACCCGAGGCGCGCGTAGATGAC
TACGGCATGAATGGCGATAAGTATAACGGCATGAAGAGGAAGAGATCCAGCGGC
AGCTTGTACTGCAGCCAGCTGCAGCTCCCAGCGGATCAGTACAGCGGCATGCTG
ATCCATCCGTTCCTCAGCCAGCAGCTGCACATGTGA
SEQ ID NO: 16: Triticum aestivum; Traes_5BL_CC18CAD72
MPMGSSAAMPALPPGFRFHPTDEELIVHYLRRQAASMPSPVPIIAEVNIYKCNPWDLP
GKALFGENEWYFFSPRDRKYPNGARPNRAAGSGYWKATGTDKAILSTPANESIGVKK
ALVFYRGKPPKGVKTDWIMHEYRLTAADNRTTKRRGSSMRLDDWVLCRIHKKCGNL
PNFSSSDQEQEHEQESSTVEDSQNNHTVSSPKSEAFDGDGDDHLQLQQFRPMAIAK
SCSLTDLLNTVDYAALSHLLLDGAGASSSDAGADYQLPPENPLIYSQPPWQQTLHYN
NNNGYVNNETIDVPQLPEARVDDYGMNGDKYNGMKRKRSSGSLYCSQLQLPADQY
SGMLIHPFLSQQLHM
SEQ ID NO: 17: sub-domain A
LPPGFRFHPTDEELICHYL
SEQ ID NO: 18: sub-domain B
IIAEVDLYKCEPWDLPEKCKI
SEQ ID NO: 19: sub-domain C
WYFFCPRDRKYPNGTRTNRATGSGYWKATGKDKEI
SEQ ID NO: 20: sub-domain D
VGMRKTLVFYMGRAPRGTKTNWVMHEFRL
SEQ ID NO: 21: sub-domain E
DEWVVCKVHHK
SEQ ID NO: 22: NAC DNA-binding domain
LPAGFRFHPTDEELMVHYLMRQAASMPCPVPIIAEVNIYQCNPWDLPAKALFGDKEW
FFFSPRDRKYPNGARPNRAAGSGYWKATGTDKAILSSSTPTSHGGANIVVGVKKALV
FYGGRPPKGTKTDWIMHEYRLSGAADDDCKGSTRRRVSSSSSSSMRLDDWVLCRIH
KK
SEQ ID NO: 23: Zmubi1 promoter
TGCAGTGCAGCGTGACCCGGTCGTGCCCCTCTCTAGAGATAATGAGCATTGCATG
TCTAAGTTATAAAAAATTACCACATATTTTTTTTGTCACACTTGTTTGAAGTGCAGTT
TATCTATCTTTATACATATATTTAAACTTTACTCTACGAATAATATAATCTATAGTACT
ACAATAATATCAGTGTTTTAGAGAATCATATAAATGAACAGTTAGACATGGTCTAAA
GGACAATTGAGTATTTTGACAACAGGACTCTACAGTTTTATCTTTTTAGTGTGCATG
TGTTCTCCTTTTTTTTTGCAAATAGCTTCACCTATATAATACTTCATCCATTTTATTA
GTACATCCATTTAGGGTTTAGGGTTAATGGTTTTTATAGACTAATTTTTTTAGTACA
TCTATTTTATTCTATTTTAGCCTCTAAATTAAGAAAACTAAAACTCTATTTTAGTTTTT
TTATTTAATAATTTAGATATAAAATAGAATAAAATAAAGTGACTAAAAATTAAACAAA
TACCCTTTAAGAAATTAAAAAAACTAAGGAAACATTTTTCTTGTTTCGAGTAGATAA
TGCCAGCCTGTTAAACGCCGTCGACGAGTCTAACGGACACCAACCAGCGAACCA
GCAGCGTCGCGTCGGGCCAAGCGAAGCAGACGGCACGGCATCTCTGTCGCTGC
CTCTGGACCCCTCTCGAGAGTTCCGCTCCACCGTTGGACTTGCTCCGCTGTCGG
CATCCAGAAATTGCGTGGCGGAGCGGCAGACGTGAGCCGGCACGGCAGGCGGC
CTCCTCCTCCTCTCACGGCACCGGCAGCTACGGGGGATTCCTTTCCCACCGCTC
CTTCGCTTTCCCTTCCTCGCCCGCCGTAATAAATAGACACCCCCTCCACACCCTCT
TTCCCCAACCTCGTGTTGTTCGGAGCGCACACACACACAACCAGATCTCCCCCAA
ATCCACCCGTCGGCACCTCCGCTTCAAGGTACGCCGCTCGTCCTCCCCCCCCCC
CCCTCTCTACCTTCTCTAGATCGGCGTTCCGGTCCATGGTTAGGGCCCGGTAGTT
CTACTTCTGTTCATGTTTGTGTTAGATCCGTGTTTGTGTTAGATCCGTGCTGCTAG
CGTTCGTACACGGATGCGACCTGTACGTCAGACACGTTCTGATTGCTAACTTGCC
AGTGTTTCTCTTTGGGGAATCCTGGGATGGCTCTAGCCGTTCCGCAGACGGGATC
GATTTCATGATTTTTTTTGTTTCGTTGCATAGGGTTTGGTTTGCCCTTTTCCTTTATT
TCAATATATGCCGTGCACTTGTTTGTCGGGTCATCTTTTCATGCTTTTTTTTGTCTT
GGTTGTGATGATGTGGTCTGGTTGGGCGGTCGTTCTAGATCGGAGTAGAATTAAT
TCTGTTTCAAACTACCTGGTGGATTTATTAATTTTGGATCTGTATGTGTGTGCCATA
CATATTCATAGTTACGAATTGAAGATGATGGATGGAAATATCGATCTAGGATAGGT
ATACATGTTGATGCGGGTTTTACTGATGCATATACAGAGATGCTTTTTGTTCGCTT
GGTTGTGATGATGTGGTGTGGTTGGGCGGTCGTTCATTCGTTCTAGATCGGAGTA
GAATACTGTTTCAAACTACCTGGTGTATTTATTAATTTTGGAACTGTATGTGTGTGT
CATACATCTTCATAGTTACGAGTTTAAGATGGATGGAAATATCGATCTAGGATAGG
TATACATGTTGATGTGGGTTTTACTGATGCATATACATGATGGCATATGCAGCATC
TATTCATATGCTCTAACCTTGAGTACCTATCTATTATAATAAACAAGTATGTTTTATA
ATTATTTTGATCTTGATATACTTGGATGATGGCATATGCAGCAGCTATATGTGGATT
TTTTTAGCCCTGCCTTCATACGCTATTTATTTGCTTGGTACTGTTTCTTTTGTCGAT
GCTCACCCTGTTGTTTGGTGTTACTTCTGCAG
SEQ ID NO: 24: ZmNac111 promoter
TCTGAAAGAAATAAATATTGCTAAGATTTTTTCTAAAATTTAGTCAAAATCAAGAAA
GTTTAACCATCATGAACATCGTAGCGACACTGATTCTAGGTATTAATTAAGTATTCC
TACAGGCTAGACGAAGCAATTAATATGAGAAAGGTTGACGGAGGTCCGGGGACG
AAGGTCATAACCCTTGGGACCATTTTAATCTCTAAAACAATAACTAGAAGCCATTTT
TATTAGTTTGACAGTTAAACTAAATTACGGTTGCACTATTCCTTCTATTGATACGAC
GCAATTCCCATGATTCTAAGCCTGGTATGTCTGGCTTTTTTCGACTCTGGTTATTAA
AAGCTGTTGCGGACTATCAAATACCTCATCTTTTCGGTCCGCTTCTATAAGAATCG
TTTTGGTAAAAACCTATTCGAAATCAACATAAACACAAAATCGGTCGAGCCGTCAC
GATAGAAGTAATCATCTCTTTCCTAAACCTTTTAGACCACTTCATGTTTCTCCACAC
GTATTGTTCACGATACTCGGTCAGATTCTCCATAAAAAAAGTTGAGCTAAACGGCC
CCTAAAGTATATGTAGATAAGCACGATTCTAGTACGACTAACCGCAGGACTTCTTG
AGCAGAGCAAAAGATGGATATTAATTACAGAAGAACTGACGGGGCGCATGGTTTC
CACGAGGTGGGCGCACCCCGGCCACATGCACCGGCGAAAAACAAAATGGCAGTC
GCGAATCGAAAGCTGCCAAGCTAGGCCGCATGCTGGGGATGTGTGACACACATA
TCATTGGCGGTCATGGCAGGTCGACACGTGTAGGCGGACGTCATCCATCGTCTTC
ATCCCTATCTATCTATCTATCACTCAGTACAAGCCATTGCTTTCGTTATCTTCCGAG
GCGGGCAGGGCAGGGCAGGTTCTTCCCCCCTTGGCACGCACGGCTTGCGTGGA
GCCAGGCCAGCTCGACGATGACTACGCGGCCCAGTCCTCCTCTTCGATCCTATAA
AAGCGAGGCCCAGTCCCTCCATCTCAAAAATTTCAATGCCTCTCTAGATCTTTTAA
GCTAGCTATCCTTGCTTCCTCCCTTCACTTTCTTATTGAGAGCTCTCTGGCGTGCT
GTACCGGACGACAGGGGTTGGTAGGTCGTAGGTGTAGCTAGCTCGGCGAGCGA
GCAATCCGGCCAACAGCACACGCGTCGATCGCC
SEQ ID NO: 25 (ZmNac111 promoter and MITE).
TCTGAAAGAAATAAATATTGCTAATATTTTTTCTAAAATTTAGTCAAAATCAAGAAAG
TTTAACCATCATGAACATCGTAGCGACACTGATTCTAGGTATTAATTATCTGCATTC
AAGTATTCCTACAGGCTAGACGAAGCAATTAATATGAGAAAGGTTGACGGAGGTC
CGGGGACGAAGGTCATAACCCTTGGGACCATTTTAATCTCTTTGTCTAAAACAATA
ACTAGAAGCCATTTCTATTAGTTTGACAGTTAAACTAAATTACGGTTGCACTATTCC
TTCTATTGATACGACGCAATTCCCATGATTCTAAGCCTGGTATGTCTGGCTTTTTTC
GACTCTGGGTATTAAAAGCTGTTGCGGACTATCAAATACCTCATCTTTTCGGTCCG
CTTCAAAACCTATTCGAAATCAACATAAACACAAAATCGGTTGAGCCGTCACGTCA
CGATAGAAGTAATCATCTCTTTCCTAAACCTTTTAGACCACTTCATGTTTCTGCACA
CGTATTGTTCACGATACTCGGTCAGATTCTCCATAAAAAAAGTTGAGCTAAACAGC
CCCTAAAGTATATGTAGATAAGCACGATTCTAGTACTAACCGCAGGACTTCTTGAG
CAGAGCAAAAGATGGATACTCCCTCCGTTTCTTTTTATTAGTCGCTGGATAGTGCA
ATTTTGCACTATCCAGCGACTAATAAAAAGAAACGGAGGGAGTATTAATTACAGAA
GAACTGACGGGGCGCATGGTTTCCACGAGGTGGGCGCACCCCGGCCACATGCAT
CGGCGAAAACAAAATGGCAGTCGCGAATCGAAAGCTGCCAAGCTAGGCCGCATG
CTGGGGATGTGTGACACACATATCATTGGCGGTCATGGCAGGTCGACACGTGTA
GGCGGACGTCATCCATCGTCTTCATCCCTATCTATCTATCTATCACTCAGTACAAG
CCATTGCTTTCGTTATCTTCCGAGGCGGGCAGGGCAGGGCAGGTTCTTCCCCCCT
TGGCACGCACCGCACGCACGGCTTGCGTGGAGCCAGGCCAGCTCGACGATGAC
TACGCGGCCCCGGCCCCGCGGCCCAGTCCTCCCTCCTCTTCGATCCTATAAAAG
CGAGGCCCAGTCCCTCCATCTCAAAAATTTCAATGCCTCTCTAGATCTTTCAAGCT
ATCCTTGCTTCCTAGCTCCCTTTGGCGTGCTGTACCGGACGACAGGGGTAGGTAG
GTAGGTAGGTGTAGCTAGCTCGGCGAGCGAGGGAGCAATCCGGCCAACAGCACA
CGCGTCGATCGCC
SEQ ID NO: 26 (MITE)
CTCCCTCCGTTTCTTTTTATTAGTCGCTGGATAGTGCAATTTTGCACTATCCAGCG
ACTAATAAAAAGAAACGGAGGGAGTA
SEQ ID NO: 27 Corn inbred (Zea Mays L.)
Met Pro Arg Ser Gly Gly Gly Gly Asn Gly Gly Gly Ile Ser Ser Ser
Ser Arg Val Met Pro Asn Leu Pro Ala Gly Phe Arg Phe His Pro Thr
Asp Glu Glu Leu Met Val His Tyr Leu Met Arg Gln Ala Ala Ser Met
Pro Cys Pro Val Pro Ile Ile Ala Glu Val Asn Ile Tyr Gln Cys Asn
Pro Trp Asp Leu Pro Ala Lys Ala Leu Phe Gly Asp Lys Glu Trp Phe
Phe Phe Ser Pro Arg Asp Arg Lys Tyr Pro Asn Gly Ala Arg Pro Asn
Arg Ala Ala Gly Ser Gly Tyr Trp Lys Ala Thr Gly Thr Asp Lys Ala
Ile Leu Ser Ser Ser Thr Pro Thr Ser His Gly Gly Ala Asn Ile Val
Val Gly Val Lys Lys Ala Leu Val Phe Tyr Gly Gly Arg Pro Pro Lys
Gly Thr Lys Thr Asp Trp Ile Met His Glu Tyr Arg Leu Ser Gly Ala
Ala Asp Asp Asp Cys Lys Gly Ser Thr Arg Arg Arg Val Ser Ser Ser
Ser Ser Ser Ser Met Arg Leu Asp Asp Trp Val Leu Cys Arg Ile His
Lys Lys Ser Asn Asp Phe Gln Leu Ser Ser Ser Ser Glu His Glu His
Glu Gln Glu Glu Pro Ala Ala Gly Gly Ser Ala Thr Thr Val Glu Glu
Leu Asp Ala Leu Val Val Val Asp Asn Ser Ser Ser Ser Asp Thr Thr
Thr Thr Thr Ile Thr Thr Asn Asp Asn Asn Ser Thr Glu Thr Ala Ala
Thr His His Asp Pro Gln Thr Met Met Met Leu Ser Lys Ser Cys Ser
Leu Thr Asp Leu Leu Asp Ser Ile Asp Tyr Ala Ala Leu Ser Ser Gln
Met Leu Leu Leu Asp Ala Pro His Pro His Ala Asp Asp Pro Pro Pro
His Met Val Tyr Tyr Pro Pro Ala Ala Ser Ala Thr His Gln Gln Ala
Ile Ile Ala Asn Asn Thr Asn Ser Asn Asp Asp Tyr Tyr Asp Asp Asp
Asn Leu Leu Pro Thr Ala Ala Ala Thr Ala Val Asp Ala Ala Ala Ala
Ala Val Leu Leu Ser Ser Ser Pro Ala Asp Thr Asn Gly Val Asn Lys
Arg Lys Arg Val Thr Ala Val Asp Tyr Tyr Gly Ala Ala Glu Pro Pro
Thr Phe Phe His His His Leu Asp Asp Gly Gly Asn Ser Phe Phe Ser
Thr Thr Lys Lys Leu Lys Pro Pro Pro Pro Ser Asp Ser Arg His Gly
Gly His Phe Gly Thr Ala Thr Ala Ser Ser Tyr Arg Asp Ser Gln Gln
Leu Val Asp Ser Ala Ser Ser Ser Gly Val Phe Phe His Gln Tyr Gly
Tyr Gly Tyr Ser Ser Ser Asn Pro Phe Leu Asn Leu Asn Gln Gln Gln
Gln Leu Leu Leu Asn Ser His Ile Gly Met Gln
SEQ ID NO: 28 Corn inbred (Zea Mays L.)

| | |
|---|---|
| aaaaatttca atgcctctct agatctttca agctatcctt gcttcctagc tccctttggc | 60 |
| gtgctgtacc ggacgacagg ggtaggtagg taggtaggtg tagctagctc ggcgagcgag | 120 |
| ggagcaatcc ggccaacagc acacgcgtcg atcgccatgc cgagaagcgg cggcggcggc | 180 |
| aatggcggtg gcattagtag tagtagtagg gtgatgccca acctcccggc cgggttccgc | 240 |
| ttccacccca cggacgagga gctcatggtg cactacctca tgaggcaggc cgcctccatg | 300 |
| ccgtgccccg tccccatcat cgccgaggtc aacatctacc agtgcaaccc ctgggatctc | 360 |
| cctgccaagg cattgttcgg cgacaaggag tggtttttct tcagcccccg ggaccgcaag | 420 |
| taccccaacg gcgcccgccc caaccgcgcc gccgggtccg ggtactggaa ggccaccggc | 480 |
| accgacaagg ccatcctgtc gtcgtccacg cccacgagcc acggcggcgc caacatcgtc | 540 |
| gtcggcgtca agaaggcgct cgtcttctac ggcggcaggc cgcccaaggg caccaagacg | 600 |
| gactggatca tgcacgagta ccgcctctcg ggcgcggcgg acgacgactg caagggcagc | 660 |
| accaggcgca gagtgtcttc ctcctcgtcg tcctccatga ggctggacga ctgggtgctg | 720 |
| tgcaggatcc acaagaagag caacgatttc cagttgtcgt cgtcgtcgga gcatgagcac | 780 |
| gagcaggagg agccggcggc gggcggctca gcgacgactg tggaggaact agacgctctc | 840 |
| gtcgtcgtcg acaacagcag cagcagcgac accaccacca ccaccatcac caccaacgac | 900 |
| aacaactcta ctgaaacagc tgcgactcat cacgatcctc agacgatgat gatgctgagc | 960 |
| aagtcgtgct cgctcaccga cctcctcgac agcatcgact acgcggcgct ctcctcgcag | 1020 |
| atgctcctcc tcgacgcgcc ccacccccac gctgatgacc cgccgccaca catggtctac | 1080 |
| tacccaccgg cggcgtcggc gacgcaccag caagcaataa tagctaataa caccaacagt | 1140 |
| aatgacgact actatgacga cgacaacttg ttgccgactg ctgctgctac tgcggtagac | 1200 |
| gcggcggcgg cggcggtgct actcagctcg tcgcctgcag acaccaacgg tgtgaacaag | 1260 |
| aggaagcgag tgacggccgt ggactactac ggcgccgctg aaccgccgac cttcttccac | 1320 |
| caccacctcg atgatggcgg caacagcttc ttcagtacga ccaagaagct caagccgccg | 1380 |
| ccgccaagtg attcgaggca tggcggccat ttcggcaccg ccacagcgag cagctaccgc | 1440 |
| gacagccagc agctcgtgga cagcgcgagc agcagcggcg tcttcttcca tcagtatggg | 1500 |
| tacggctaca gcagcagcaa cccgttcttg aacttgaacc agcagcagca gctgctactc | 1560 |
| aacagccaca tcgggatgca gtagagctcg agagatcgag agagagagag agagagagat | 1620 |
| gatatatcat tggaaggatc agcacgcgta cgtgcagatt aattgcagcg agttctatct | 1680 |
| tgtttgcttt cattattata tactccgtat gtgtcgttca tgtaaaatta gtttaaattg | 1740 |
| taaattagtg tgctgaaatt catgtgcctg ctctctgctc atcaatgaat ggatgatgac | 1800 |
| gacccatgca cgttgcttgt gtgg | |

1. A genetically altered plant expressing a nucleic acid construct comprising a nucleic acid wherein said nucleic acid sequence encodes a polypeptide comprising SEQ ID NO: 4 or a functional homologue or variant thereof, and wherein preferably said nucleic acid is defined in SEQ ID NO: 1, 2 or 3 or a functional homologue or variant thereof.

2. (canceled)

3. A plant according to claim 1 wherein said functional homologue or variant has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the sequence represented by SEQ ID NO: 1, 2, 3 or 4.

4. A plant according to claim 1 wherein said construct further comprises a regulatory sequence.

5. A plant according to claim 4 wherein said regulatory sequence is a constitutive promoter, a strong promoter, an inducible promoter, a stress inducible promoter or a tissue specific promoter wherein preferably said regulatory sequence is a stress inducible promoter.

6. (canceled)

7. A plant according to claim 1 wherein said plant is a monocot or dicot plant.

8. A plant according to claim 7 wherein said plant is a crop plant or biofuel plant, wherein said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar, wherein more preferably said crop plant is maize.

9. (canceled)

10. (canceled)

11. A plant according to claim 1 wherein said plant has increased drought tolerance.

12. A product derived from a plant as defined in claim 1 or from a part thereof.

13. A vector comprising a nucleic acid as defined in SEQ ID NO: 1, 2 or 3 or a functional homologue or variant thereof.

14. A vector according to claim 13 wherein said vector is an expression vector wherein the vector further comprises a regulatory sequence which directs expression of the nucleic acid, wherein preferably said regulatory sequence is a constitutive promoter, a strong promoter, an inducible promoter, a stress inducible promoter or a tissue specific promoter and wherein more preferably, said regulatory sequence is a stress inducible promoter.

15. (canceled)

16. (canceled)

17. (canceled)

18. A host cell comprising a vector according to claim 13, wherein preferably said host cell is a bacterial or a plant cell.

19. (canceled)

20. (canceled)

21. (canceled)

22. (canceled)

23. A method for increasing yield of a plant under drought or water deficit conditions or increasing drought tolerance of a plant said method comprising introducing and expressing in said plant a nucleic acid construct comprising nucleic acid as defined in SEQ ID NO: 1, 2 or 3 or a functional homologue or variant thereof.

24. (canceled)

25. A method according to claim 23 wherein said nucleic acid sequence encodes a polypeptide comprising SEQ ID NO: 4 or a functional homologue or variant thereof, wherein preferably said functional homologue or variant has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the sequence represented by SEQ ID NO: 1, 2, 3 or 4.

26. (canceled)

27. A method according to claim 23 wherein said construct further comprises a regulatory sequence, wherein said regulatory sequence is a constitutive promoter, a strong promoter, an inducible promoter, a stress inducible promoter or a tissue specific promoter, wherein more preferably said regulatory sequence is a stress inducible promoter.

28. (canceled)

29. (canceled)

30. A method according to claim 23 wherein said plant is a monocot or dicot plant.

31. A method according to claim 30 wherein said plant is a crop plant or biofuel plant, wherein preferably said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

32. (canceled)

33. A method according to claim 31 wherein said crop plant is maize.

34. (canceled)

35. A method for producing a mutant plant tolerant to drought comprising introducing a mutation into the nucleic acid sequence of the endogenous ZmNAC111 promoter or a functional homologue or variant thereof using targeted genome modification.

36. The method of claim 35, wherein the mutation is introduced using ZFNs, TALENs or CRISPR/Cas9.

37. A genetically altered plant wherein said plant carries a mutation in the endogenous NAC111 promoter.
